SEQUENCE LISTING (20 sequences)

SEQ ID NO: 1 - DNA, 20 bp, Homo sapiens
tggcttttcagctctgcatc

SEQ ID NO: 2 - DNA, 21 bp, Homo sapiens
ccgctacatctgaatgacctg

SEQ ID NO: 3 - DNA, 20 bp, Homo sapiens
gaggcccccaaaaaggagtc

SEQ ID NO: 4 - DNA, 20 bp, Homo sapiens
aggttcccatcggccacata

SEQ ID NO: 5 - DNA, 20 bp, Homo sapiens
ctgccacatagcccagttca

SEQ ID NO: 6 - DNA, 25 bp, Homo sapiens
agaagcgactcttctaaggcatctt

SEQ ID NO: 7 - DNA, 22 bp, Homo sapiens
ggctgtttaccagactccgaca

SEQ ID NO: 8 - DNA, 22 bp, Homo sapiens
cacaaagcctggcagctctcta

SEQ ID NO: 9 - DNA, 21 bp, Homo sapiens
ggaatacacaacctactagcc

SEQ ID NO: 10 - DNA, 20 bp, Homo sapiens
ccaggtcaccagactcctca

SEQ ID NO: 11 - DNA, 19 bp, Homo sapiens
gaaggcagctcacgaaacc

SEQ ID NO: 12 - DNA, 18 bp, Homo sapiens
aggcctcagcctcttgtg

SEQ ID NO: 13 - DNA, 24 bp, Homo sapiens
ttctgcctgcaccactcttcacga

SEQ ID NO: 14 - DNA, 25 bp, Homo sapiens
gccagtcttcagagctgtgcctttg

SEQ ID NO: 15 - DNA, 23 bp, homo sapiens
ccgaactgtcctgggcctgatcc

SEQ ID NO: 16 - DNA, 23 bp, Homo sapiens
cccattccccaggtcccatgtgg

SEQ ID NO: 17 - DNA, 20 bp, Homo sapiens
gacgaaggcttcaccactgt

SEQ ID NO: 18 - DNA, 20 bp, Homo sapiens
gtcaagtggatgtctcgtgc

SEQ ID NO: 19 - DNA, 20 bp, Homo sapiens
agaaggctggggctcatttg

SEQ ID NO: 20 - DNA, 20 bp, Homo sapiens
aggggccatccacagtcttc

(19) United States
(12) Patent Application Publication
(10) Pub. No.: US 2024/0024337 A1
(43) Pub. Date: Jan. 25, 2024

(54) INTERACTION OF SARS-COV-2 PROTEINS WITH MOLECULAR AND CELLULAR MECHANISMS OF HOST CELLS AND FORMULATIONS TO TREAT COVID-19

(71) Applicants: Shreema Merchant, Surrey (CA); Manit Patel, Surrey (CA); AKSEERA PHARMA CORP., Vancouver (CA)

(72) Inventors: Shreema Merchant, Surrey (CA); Manit Patel, Surrey (CA); Robin Elaine Duncan, Waterloo (CA); Maria Fernanda de Andrade Fernandes, Kitchener (CA); Vishal Anant Jadhav, Mumbai (IN)

(21) Appl. No.: 18/016,762

(22) PCT Filed: Jul. 19, 2021
(86) PCT No.: PCT/IN21/50699

§ 371 Date: Jan. 18, 2023

Related U.S. Application Data
(63) Continuation of application No. PCT/IN2021/050325, filed on Mar. 30, 2021.

(30) Foreign Application Priority Data
Jul. 18, 2020 (IN) 202021030633
Dec. 12, 2020 (IN) 202021054151

(51) Int. Cl. A61K 31/00; A61P 31/14

(52) U.S. Cl. CPC A61K 31/658; A61P 31/14

(57) ABSTRACT

The present invention provides pharmaceutical compositions and methods of treating Covid-19 infectious disease. The present invention also provides pharmaceutical compositions and methods of prophylaxis or prophylactic treatment of Covid-19 infectious disease. The said methods involve administering compositions comprising therapeutically effective amount of Cannabidiol thereby causing enhancement/augmentation of innate immunity of the patient/mammal/human due to at least one of the following effects, i) infected patient cells undergo apoptosis early after infection; ii) induction of interferon transcription in the patient; iii) induction of interferon-induced antiviral effectors in the patient.

### FIELD OF THE INVENTION

The present invention provides pharmaceutical compositions and methods of treating Covid-19 infectious disease. The present invention also provides pharmaceutical compositions and methods of prophylaxis or prophylactic treatment of Covid-19 infectious disease.

### SUMMARY OF THE INVENTION

Treatment or prophylaxis of Covid-19 infectious disease is extremely challenging. This is more so because SARS-CoV-2 has many variants and some of the variants have
i) increased transmissibility,
ii) increased virulence, and
iii) reduced effectiveness of vaccines.

Under the first aspect, the present invention provides pharmaceutical compositions and methods for treating Covid-19 infectious disease comprising administering to patient such pharmaceutical compositions comprising therapeutically effective amount of a Cannabinoid wherein such administration of said pharmaceutical composition to the said patient produces an enhancement/augmentation of innate immunity of the patient due to at least one of the following effects,
i) infected patient cells undergo apoptosis early after infection;
ii) induction of interferon transcription in the patient;
iii) induction of interferon-induced antiviral effectors in the patient.

Under the second aspect, the present invention also provides pharmaceutical compositions and methods for prophylaxis or prophylactic treatment of Covid-19 infectious disease comprising administering to mammal/human such pharmaceutical compositions comprising therapeutically effective amount of a Cannabinoid wherein such administration of said pharmaceutical composition to the said mammal/human produces an enhancement/augmentation of innate immunity of the patient due to at least one of the following effects,
i) induction of interferon transcription in the patient;
ii) induction of interferon-induced antiviral effectors in the patient.

Under the third aspect, the invention provides a pharmaceutical composition and method of administering pharmaceutical composition comprising therapeutically effective amount of a Cannabinoid for preventing or reducing mutation of Sars-Cov-2 virus in a patient by administration of said pharmaceutical composition to the said patient suffering from Covid-19 by causing infected patient cells to undergo apoptosis early after infection which renders them not available to the virus for mutation.

Under the fourth aspect, the invention provides a pharmaceutical composition and methods for administering pharmaceutical composition comprising therapeutically effective amount of a Cannabinoid for use in preventing or better preparing for Covid-19 infectious disease in mammals/humans who are about to get infected wherein administration of said pharmaceutical composition to the mammal/human produces an enhancement/augmentation of innate immunity in such mammal/human due to at least one of the following effects,
i) induction of interferon transcription in the mammal/human;
iii) induction of interferon-induced antiviral effectors in the mammal/human; wherein such induction is not associated with apoptosis of cells in the beginning enabling cells to get primed/prepared for viral threat and wherein such cells are better able to prepare for infection including increase in the infectious dose of virions than usual dose and wherein the cells undergo apoptosis early after infection which renders the cells not available to the virus for replication and/or mutation.

### BRIEF DESCRIPTION OF FIGURES

FIGS. 1-5 depict cell proliferation rates which were measured by incorporating and quantifying bromodeoxyuridine (BrdU) into DNA of actively proliferating cells. The absorbance values are measured by ELISA assay with a BioTek Synergy H1 Hybrid Multi-Mode Microplate reader assay at 370 nm (reference wavelength: approx. 492 nm).

HEK293 (human embryonic kidney) cells were seeded in 96 well plates, then transfected with plasmids expressing an empty control vector (pCMV-3Tag-3a) or vectors expressing the viral Orf8, Orf10 or M proteins. Untransfected control cells have also been tested, but did not differ significantly from pCMV controls.

A few hours later the cells were treated with 1 μM of the cannabinoid, then grown for 24 hours, and assayed using a colorimetric ELISA that detects BrdU incorporation.

The inventors have run 2-way ANOVA. This has been tested in multiple separate assays on different days/weeks, with n=5 to 6 biological replicates (where separate passages of cells were considered different biological replicates). Each biological replicate was seeded in 2 to 6 technical replicates per plate, and those were averaged at each trial to give n=1 for that biological replicate in that trial.

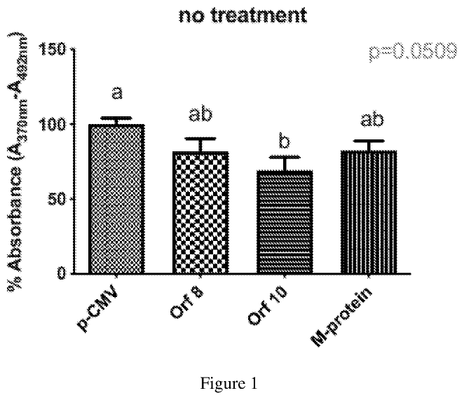

FIG. 1 provides a “no treatment” condition where HEK293 (human embryonic kidney) cells are transfected with plasmids expressing an empty control vector (pCMV-3Tag-3a) or vectors expressing the viral Orf8, Orf10 or M proteins.

Untransfected control cells (not shown in the figure) have also been tested but did not differ significantly from pCMV controls.

The viral plasmids appear to cause only a minor decrease in cell proliferation (or, possibly increases in cell death, or both). This minor decrease which is even less considering error bars are not significant to conclude impact of viral plasmids on cell proliferation. These data were not normalized to account for differences in the number of cells per well, hence it is essential to do normalization before any conclusions can be drawn from these data.

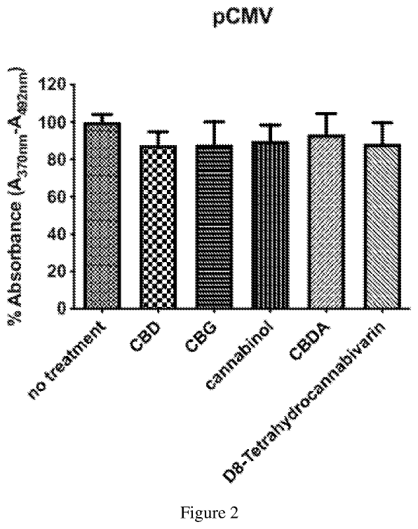

FIG. 2 provides a “control” condition where HEK293 (human embryonic kidney) cells are transfected with plasmids expressing an empty control vector (pCMV-3Tag-3a) and further treated with Cannabidiol, Cannabigerol (CBG), Cannabinol, Cannabidiolic acid and D8. Tetrahydrocannabivarin.

Cannabinoids did not significantly affect the incorporation of BrdU into cells transfected with the control plasmid.

They also did not affect the growth of untransfected control cells, or cells transfected with another control vector, pEGFP-N1 (data not shown in this figure).

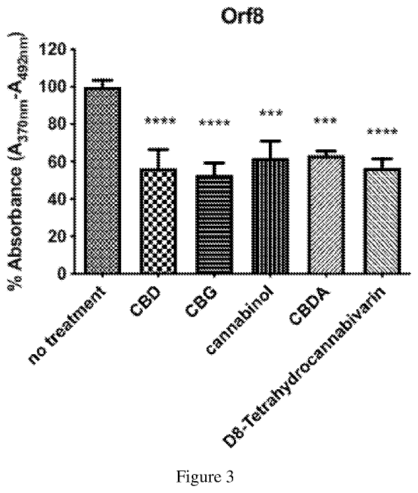

FIG. 3 provides a condition where HEK293 (human embryonic kidney) cells are transfected with plasmids expressing the viral Orf8 protein and are further treated with Cannabidiol, Cannabigerol (CBG), cannabinol, CBDA and D8-Tetrahydrocannabivarin.

Surprisingly, significant reduction in mean cell proliferation was observed, although since this data was not normalized to the number of cells that were present in the well, no conclusions can be drawn. This decrease could have been due to a decrease in cell proliferation, or a decrease in cell number, or both.

In cells expressing Orf8, BrdU incorporation was significantly reduced by treatment with any cannabinoid relative to untreated cells. This could reflect a significant decrease in mean cell proliferation, or the same rate of cell proliferation, but a decrease in cell number. Analysis is by 1-way ANOVA with Tukey's multiple comparison's test, where columns with different superscripts are significantly different, ***P<0.001, ****P<0.0001.

In cells expressing Orf8 and treated with CBD, mean BrdU incorporation was 43.52% lower than in cells expressing Orf8 but untreated with cannabinoids.

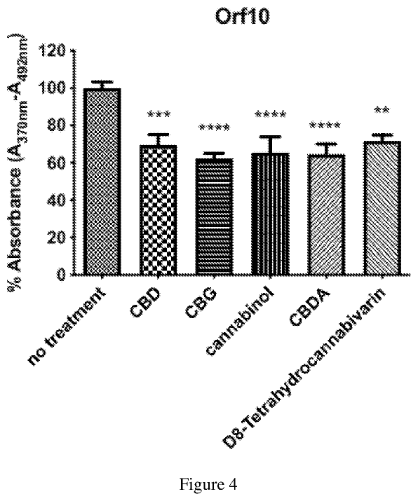

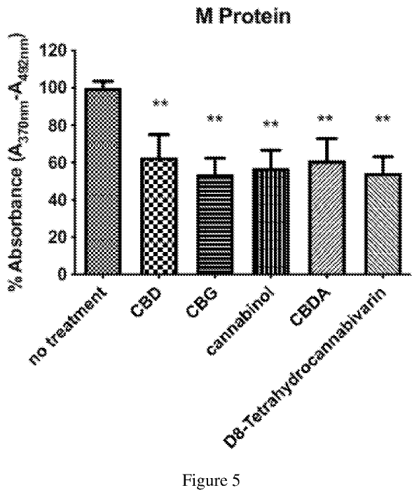

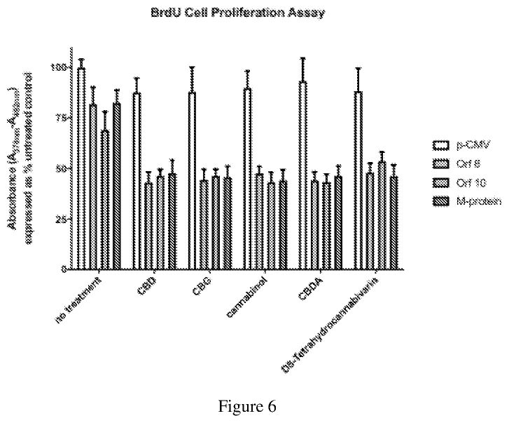

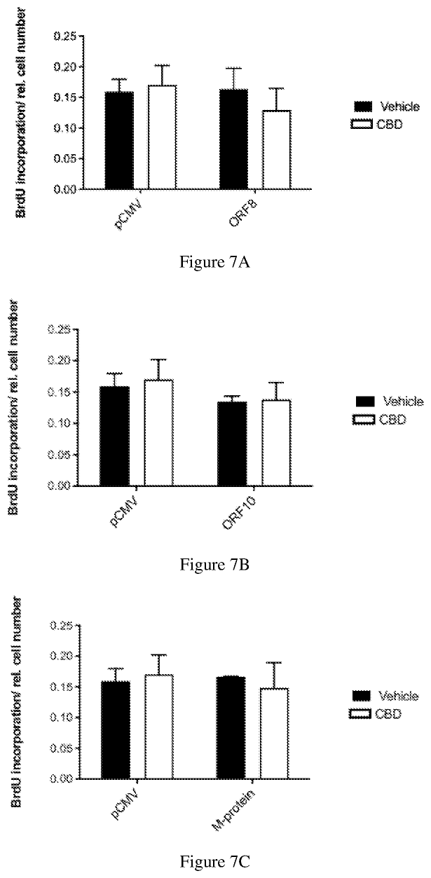

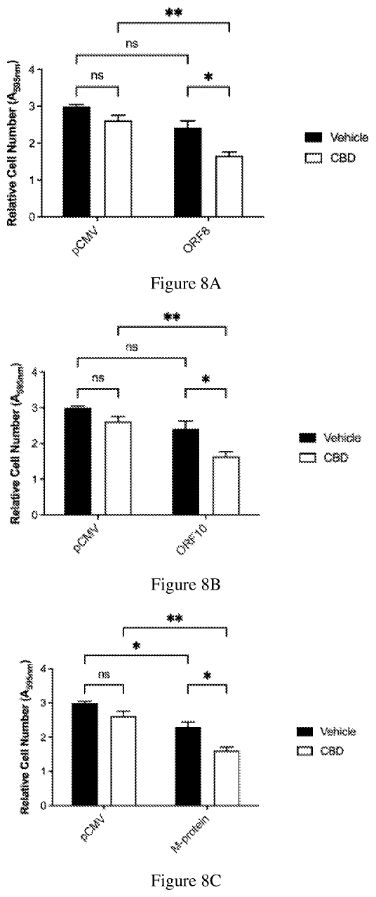

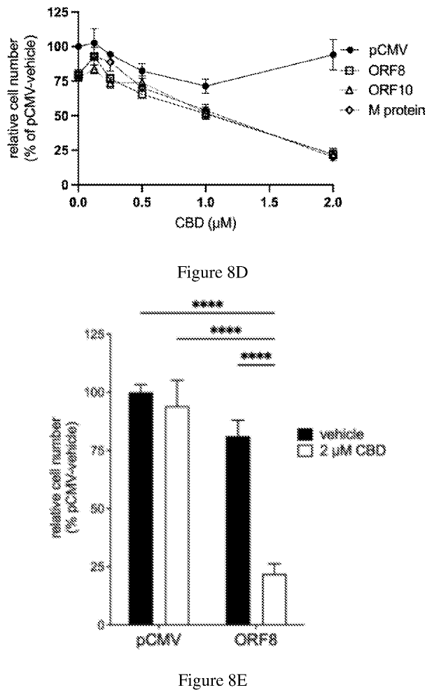

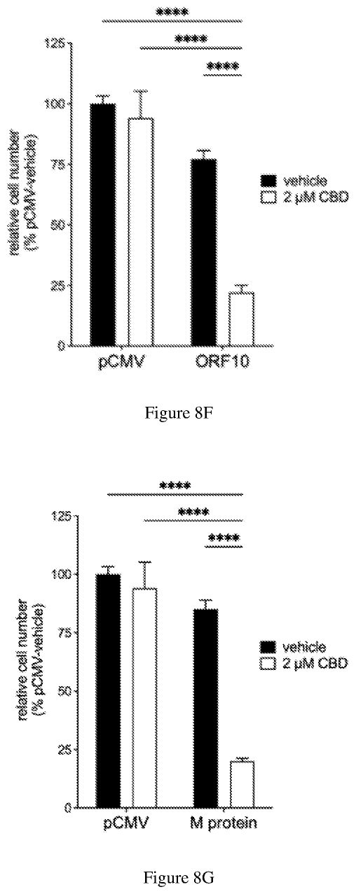

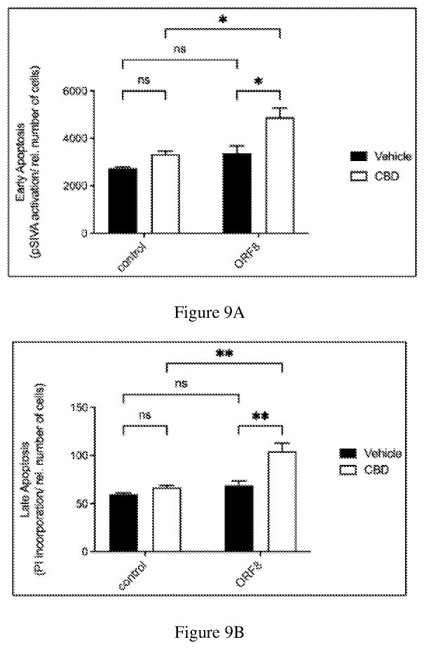

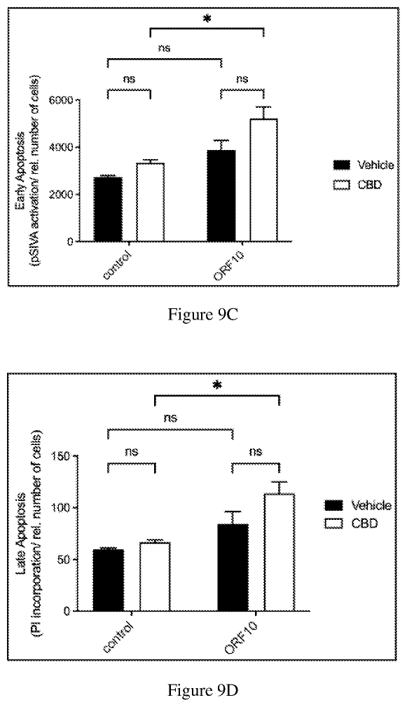

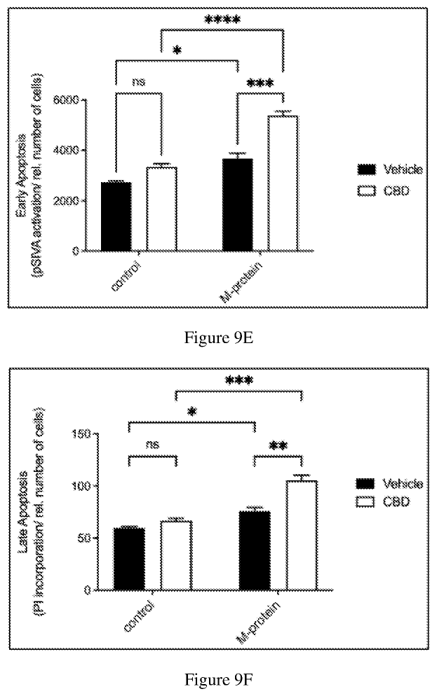

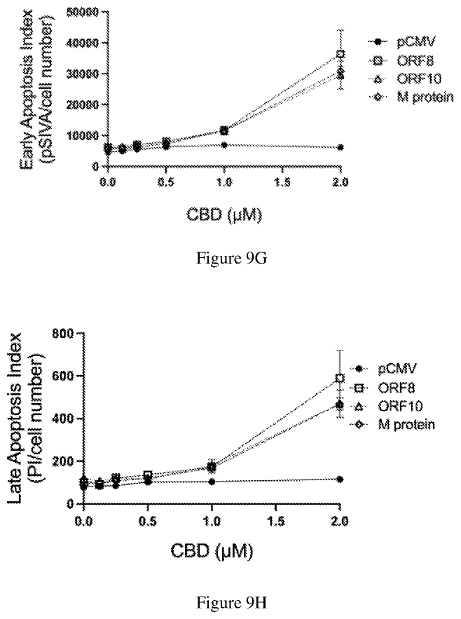

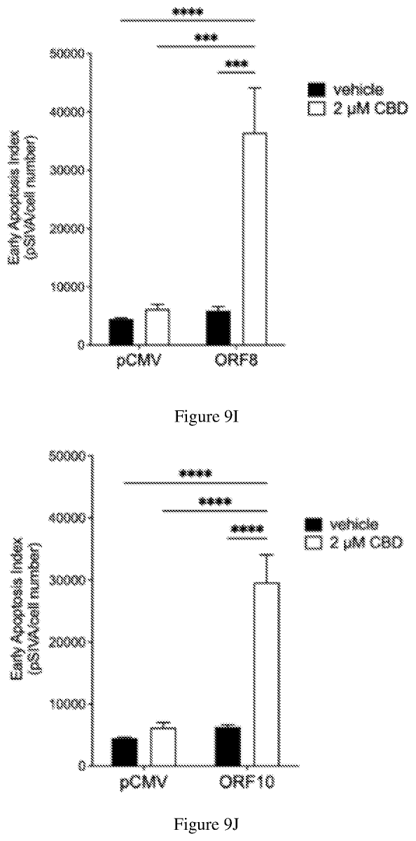

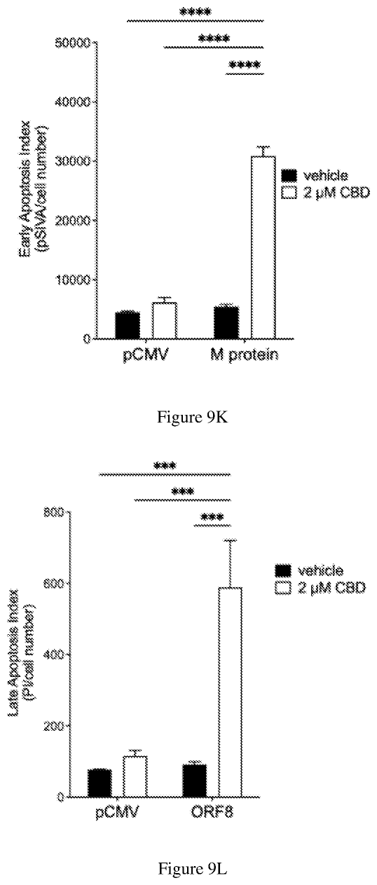

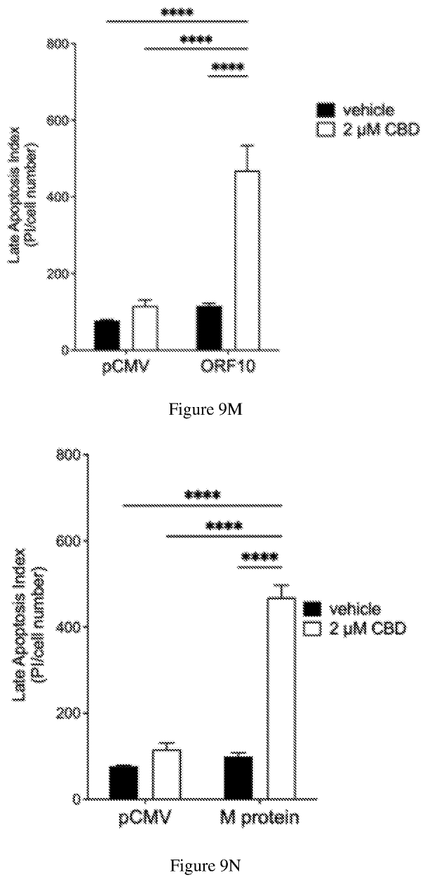

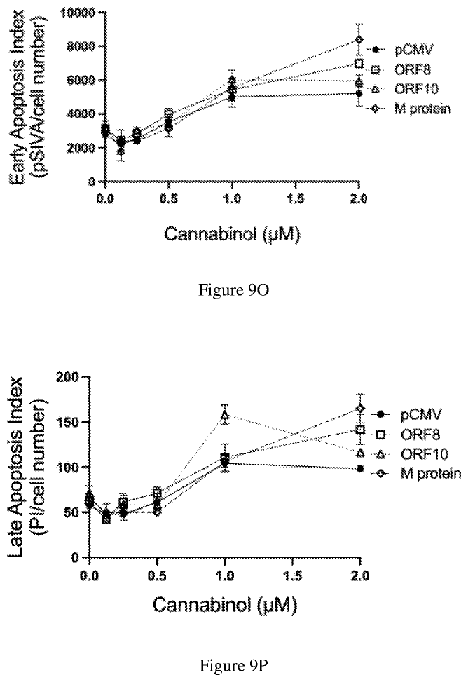

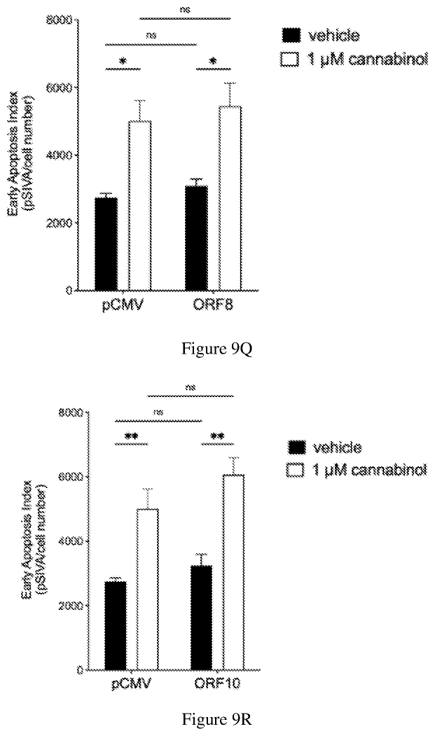

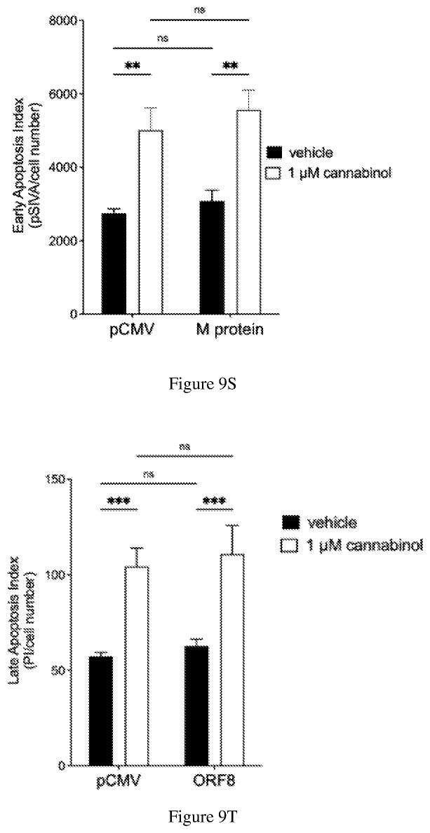

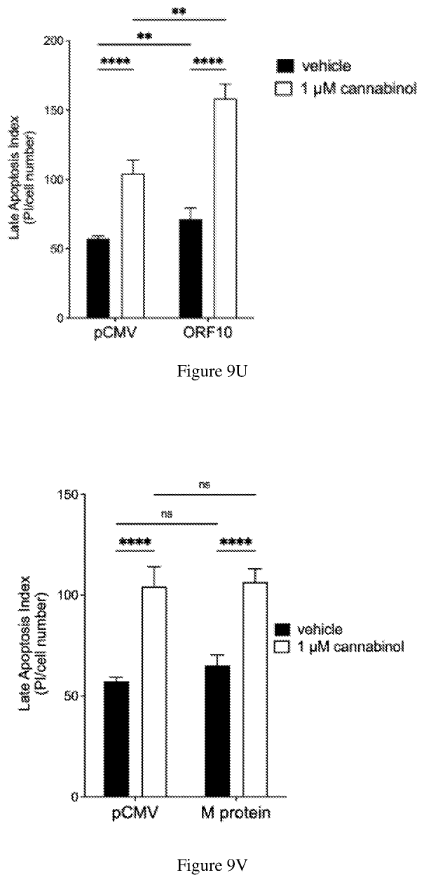

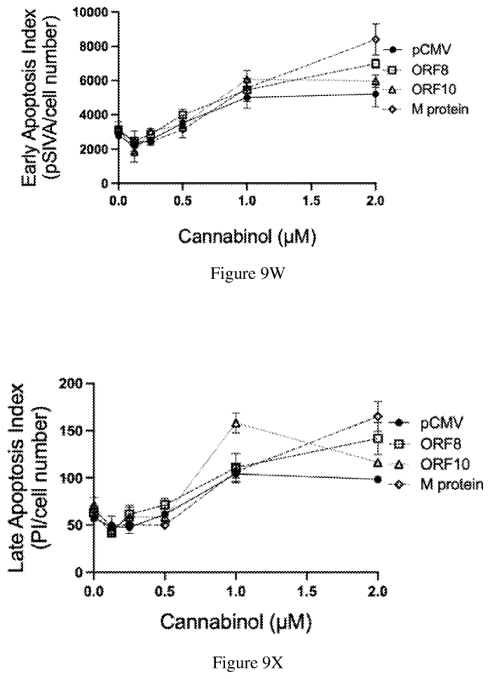

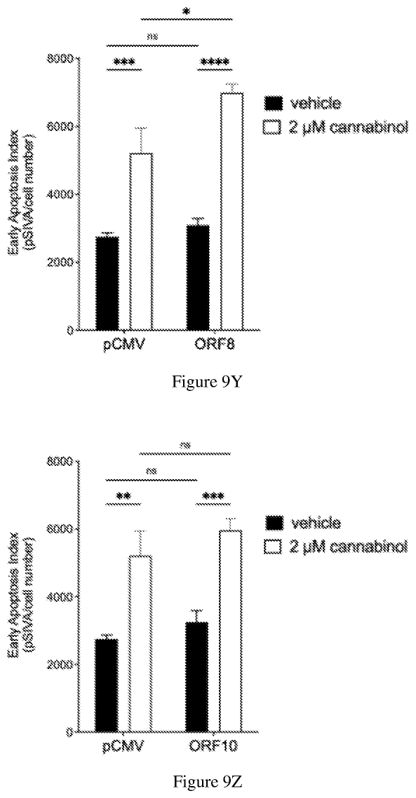

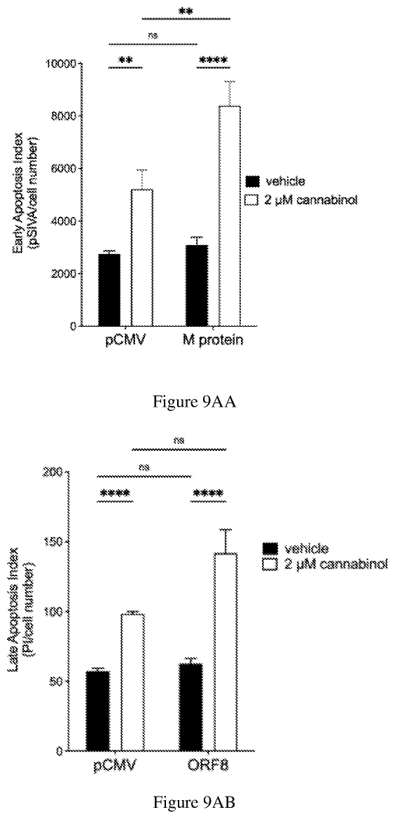

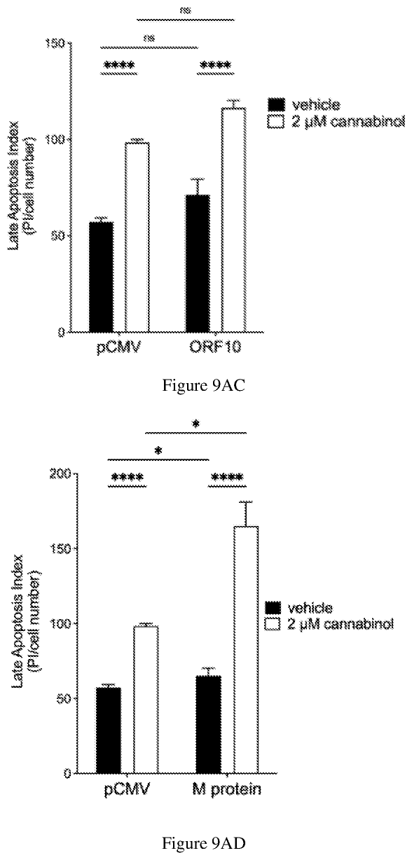

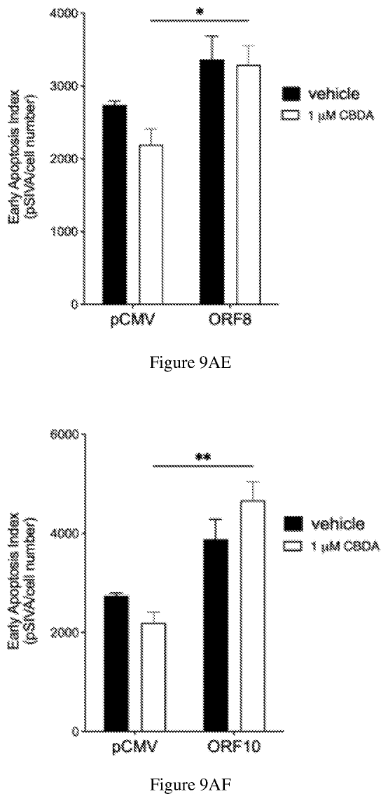

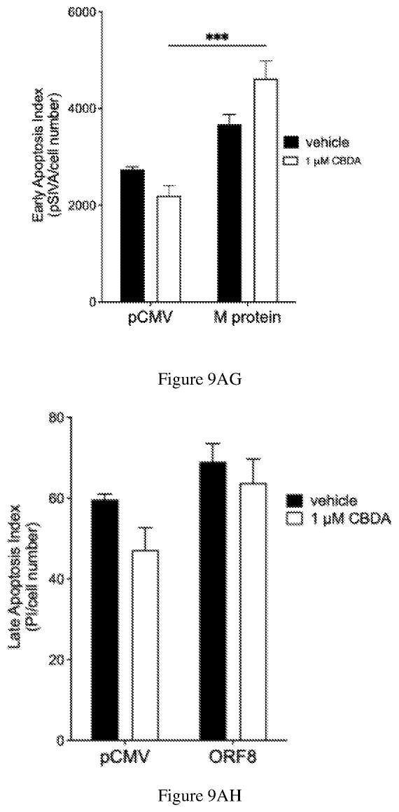

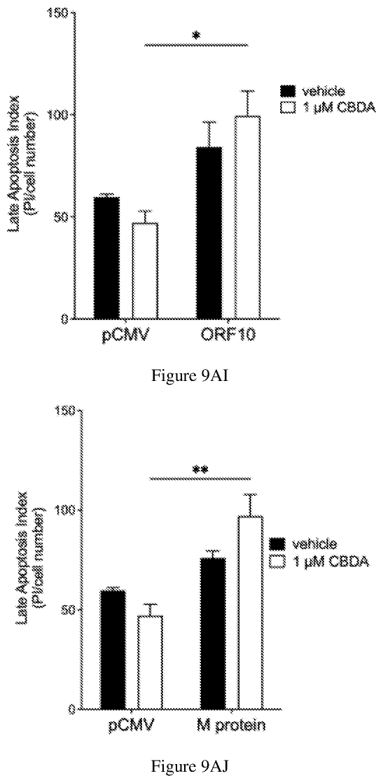

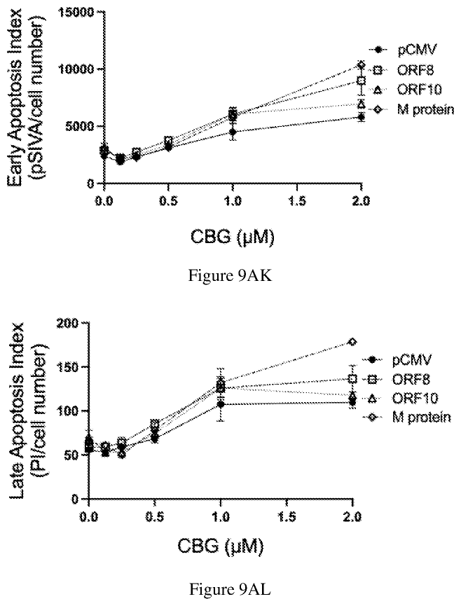

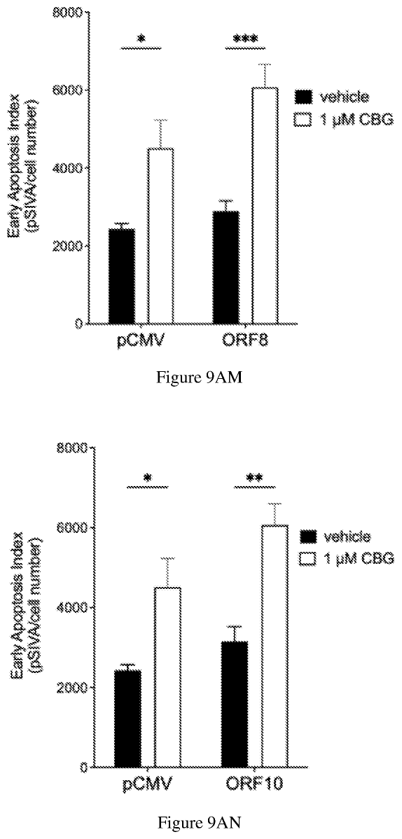

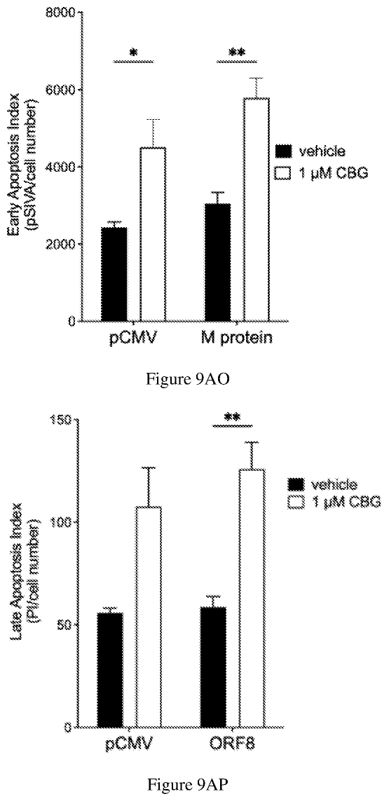

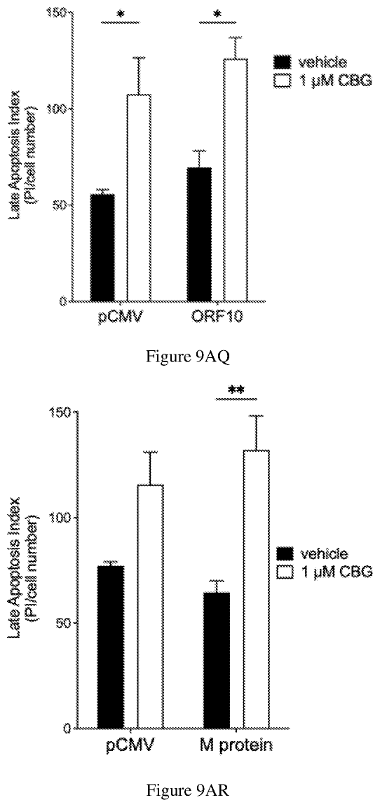

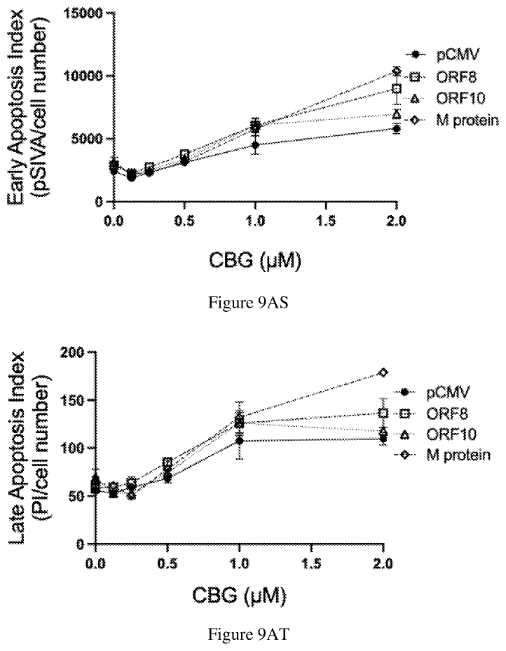

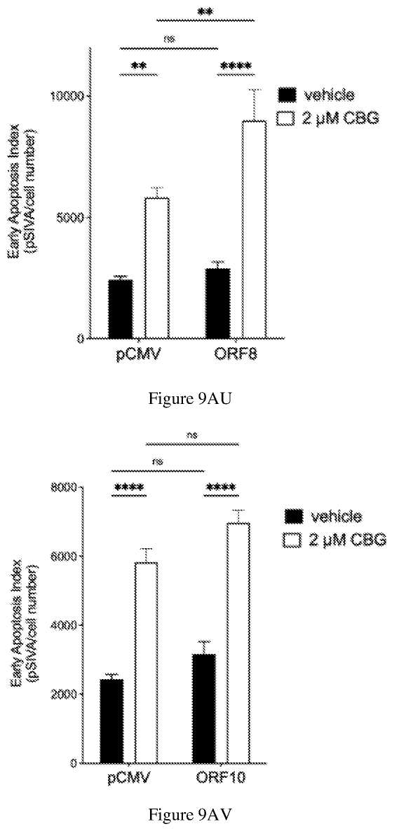

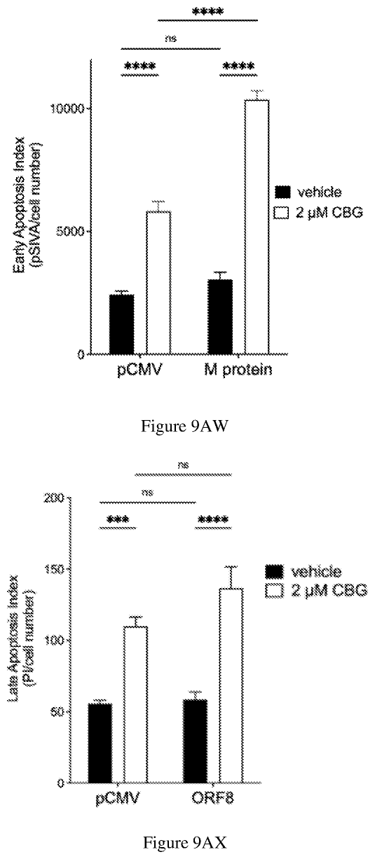

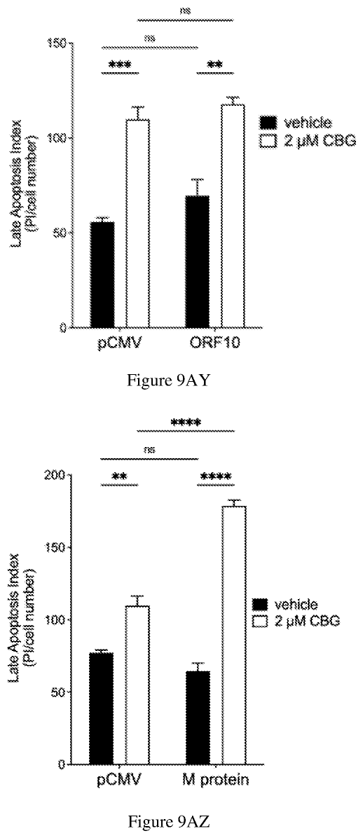

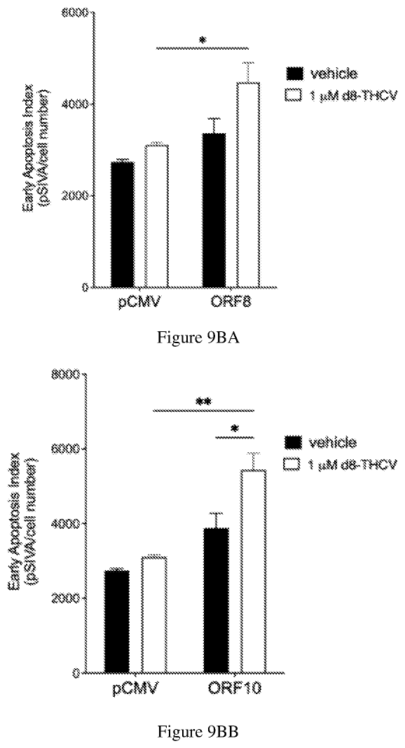

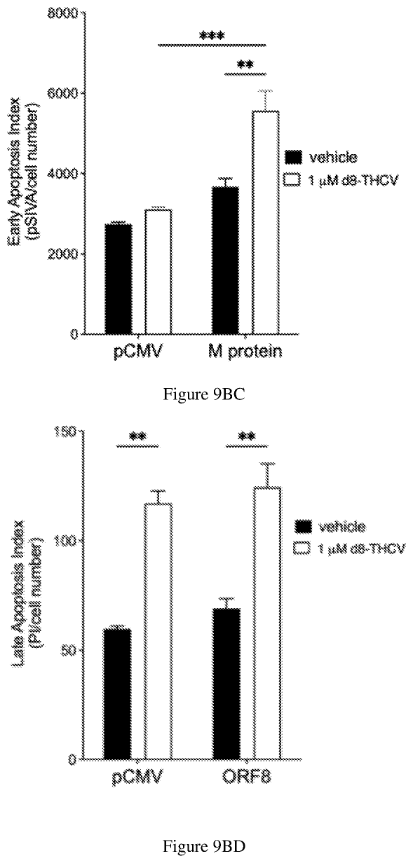

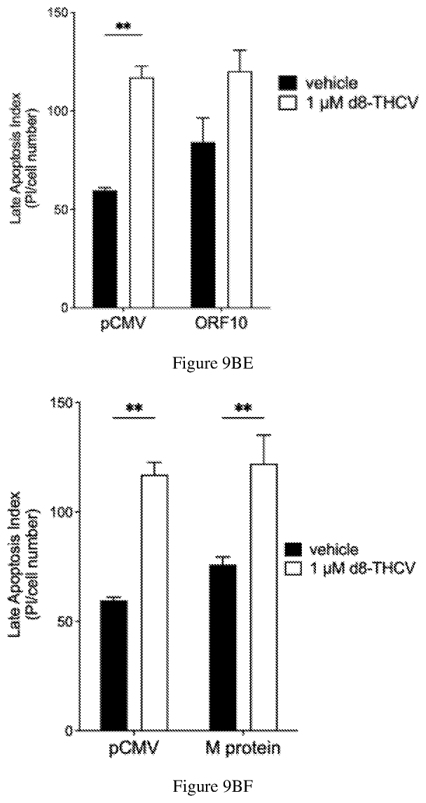

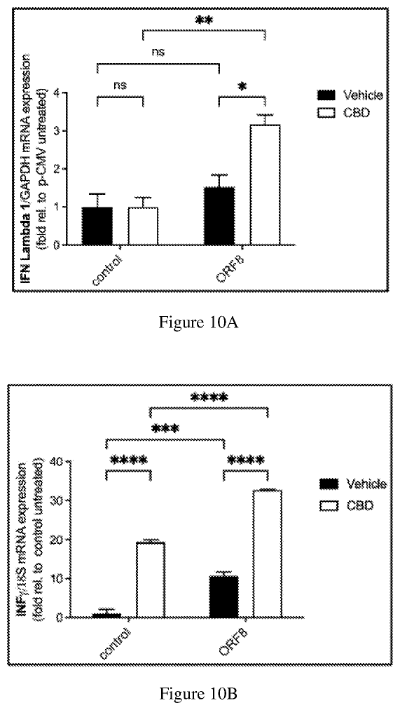

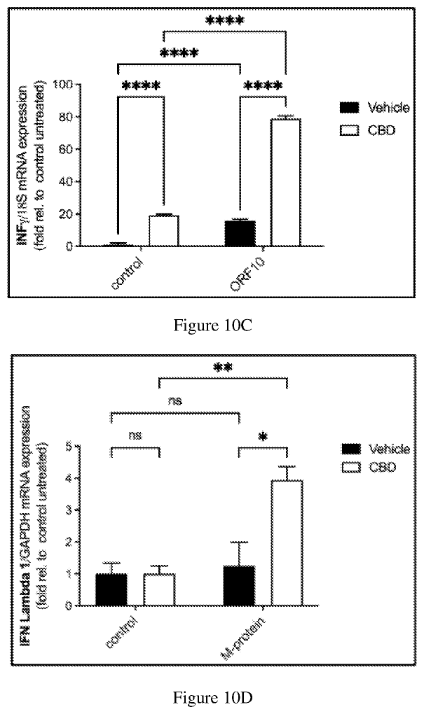

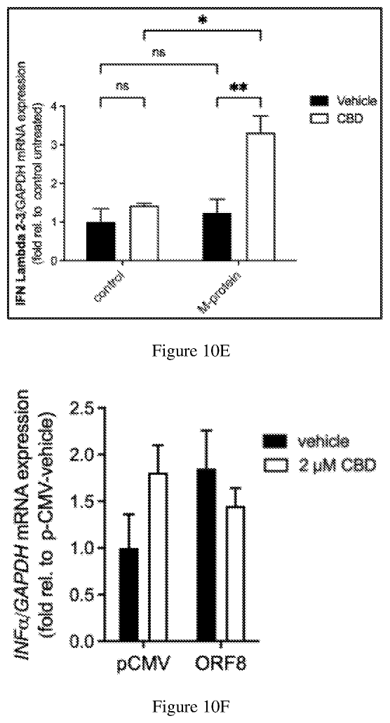

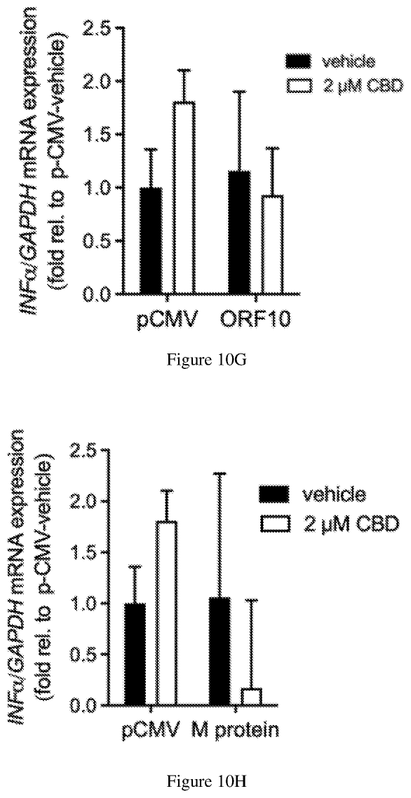

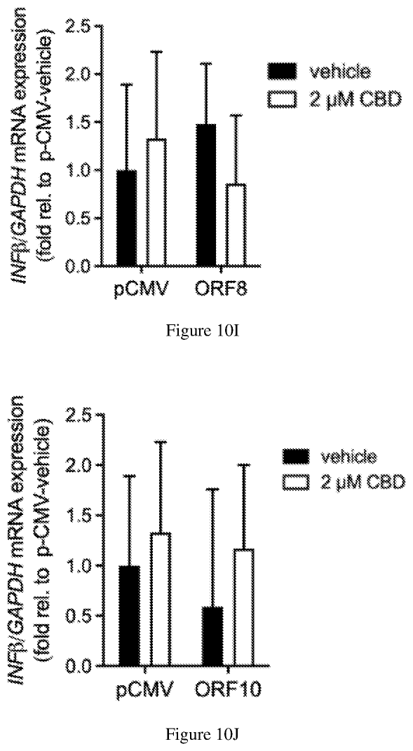

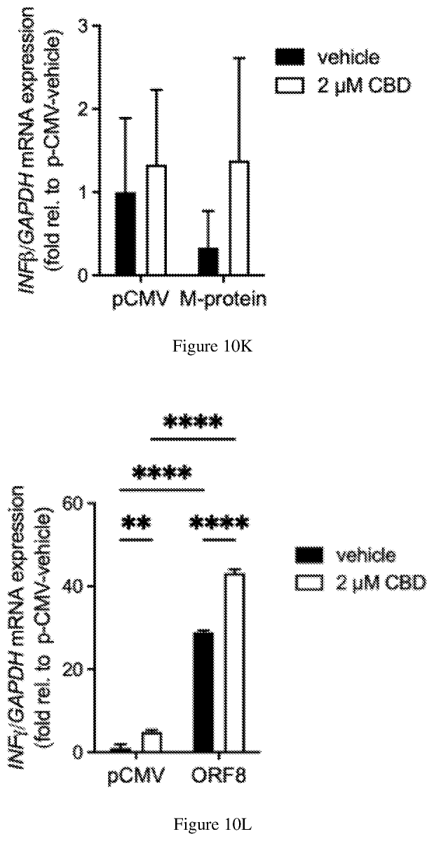

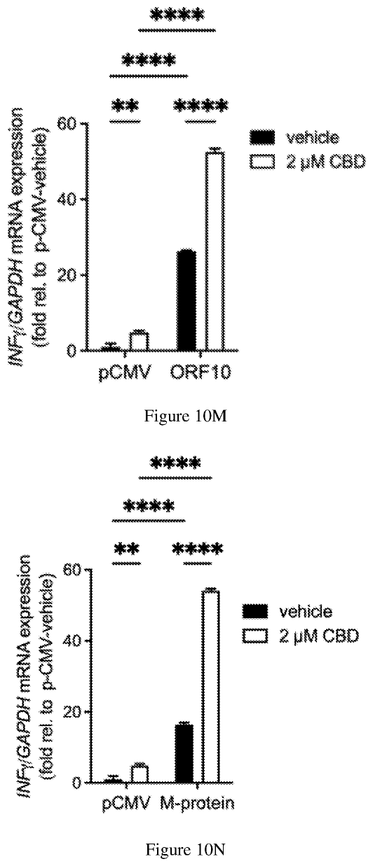

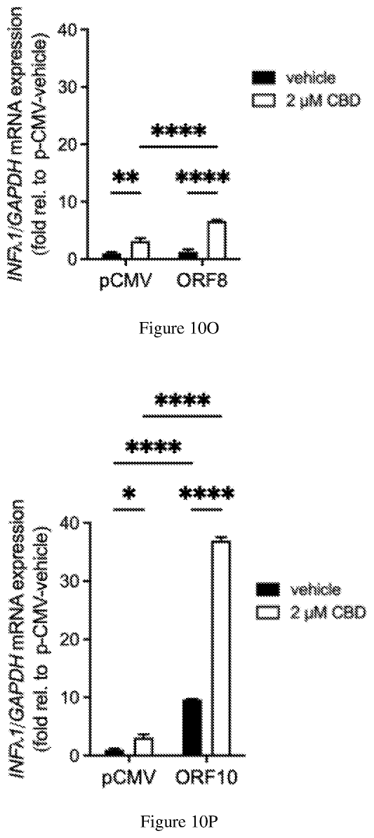

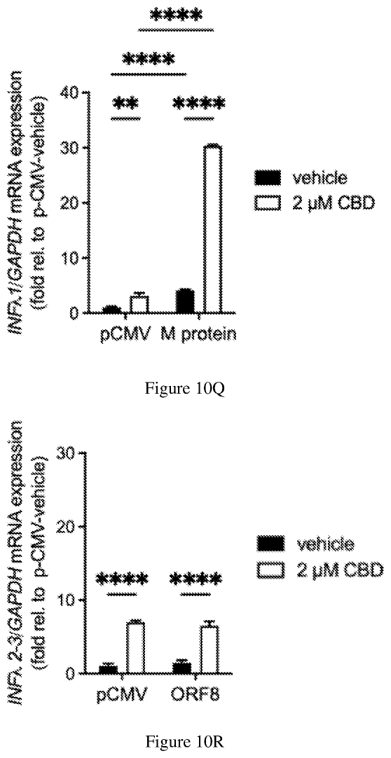

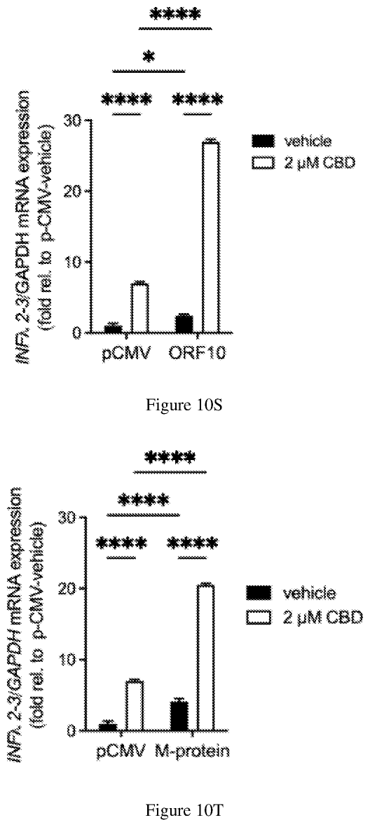

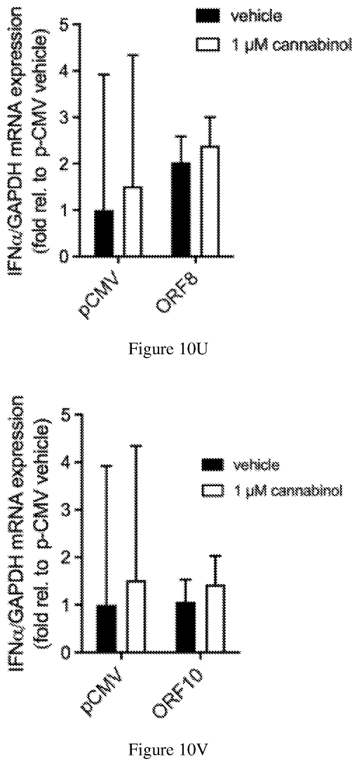

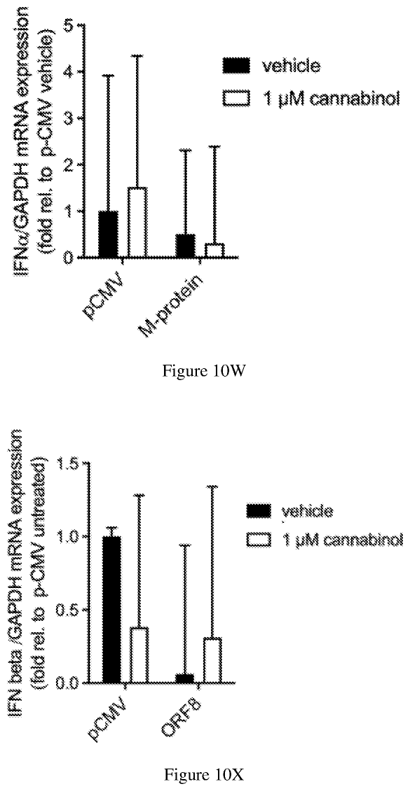

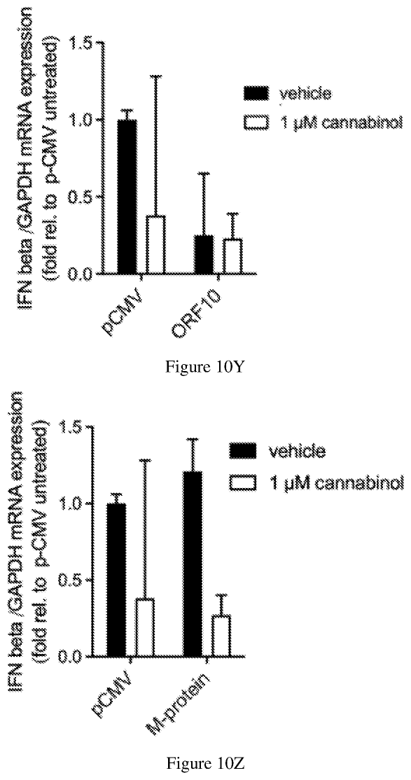

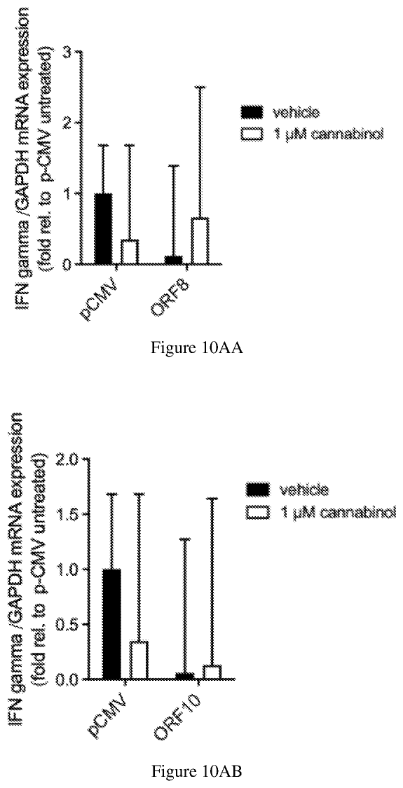

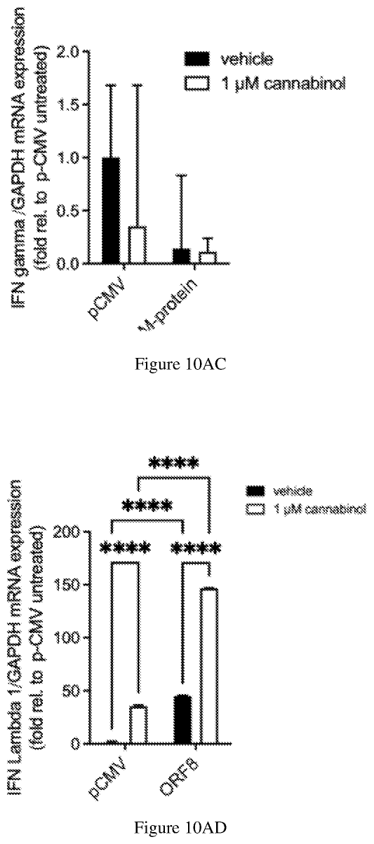

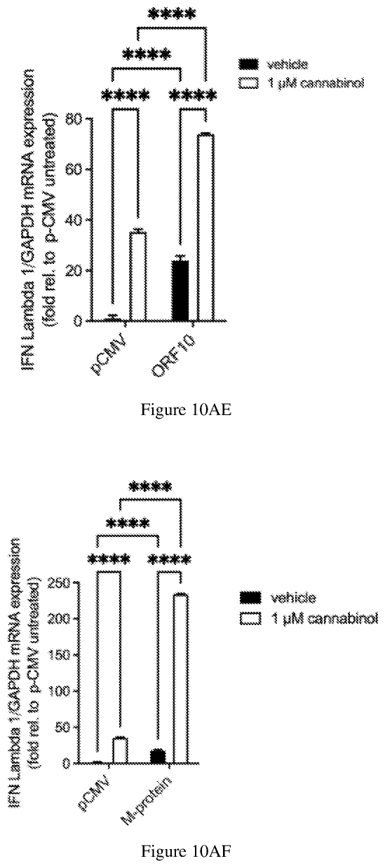

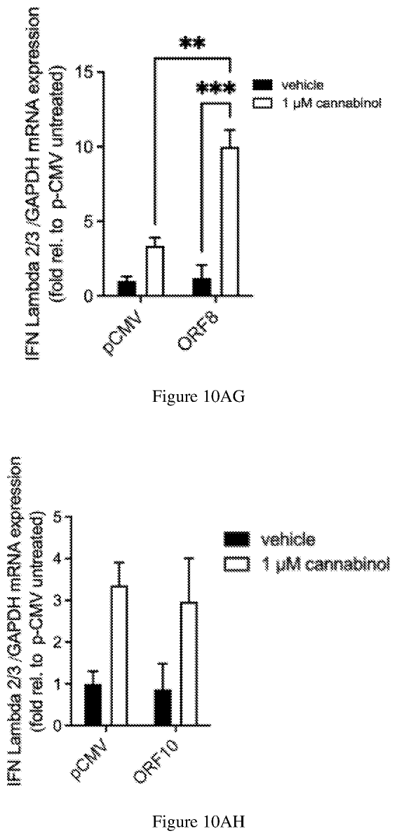

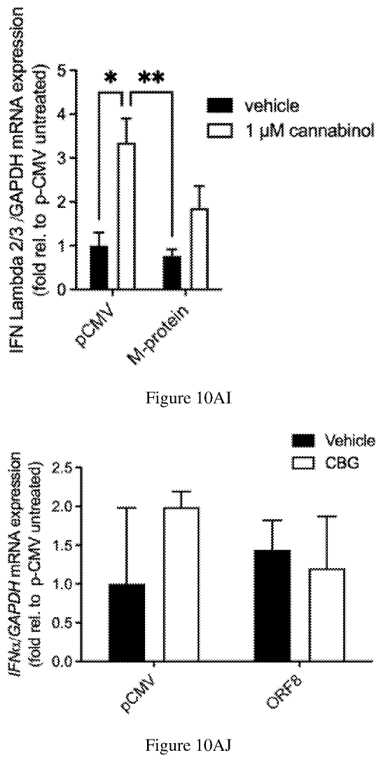

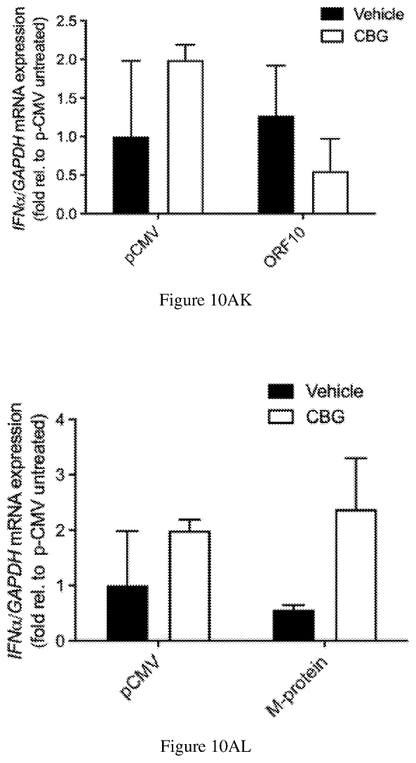

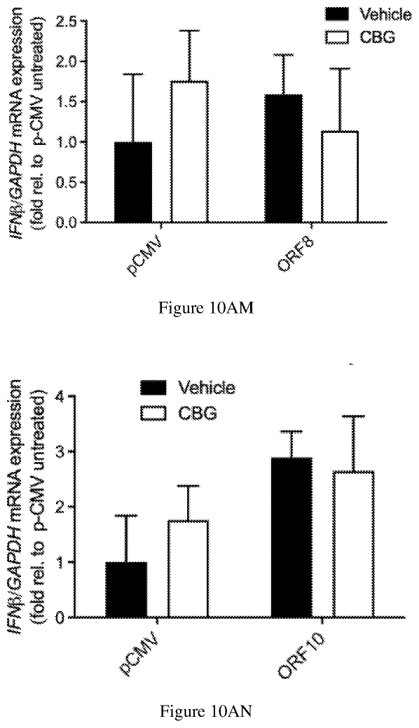

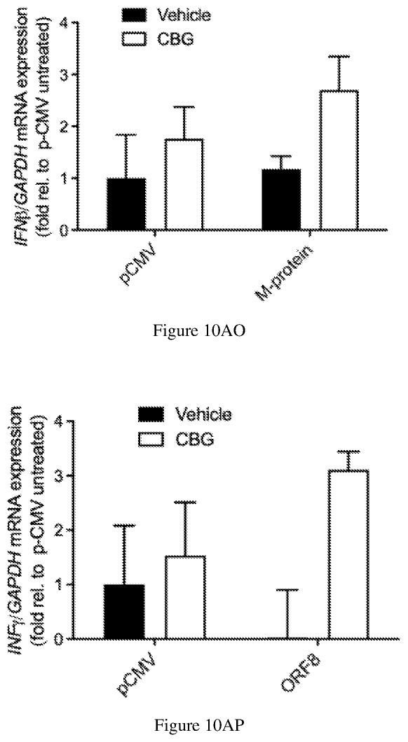

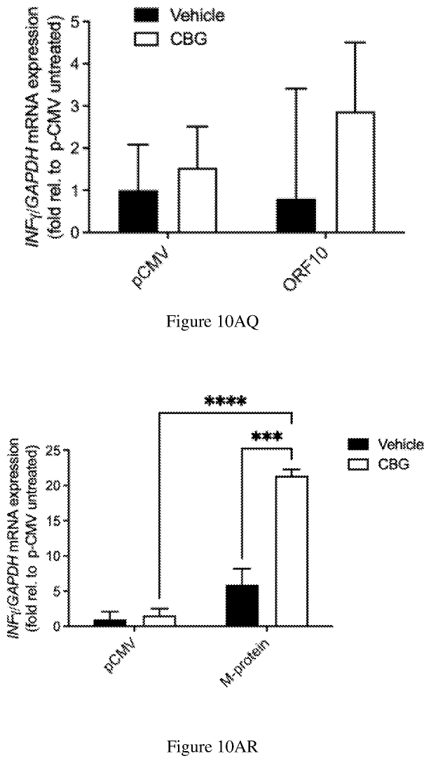

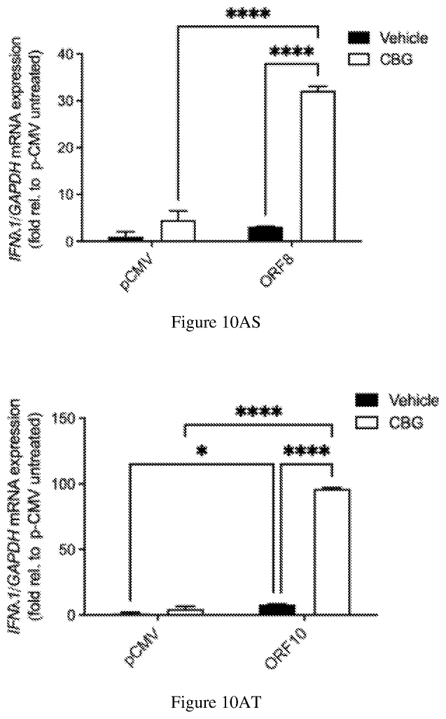

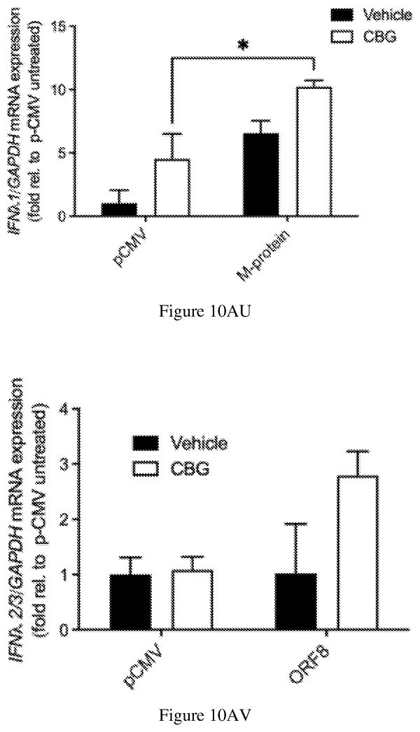

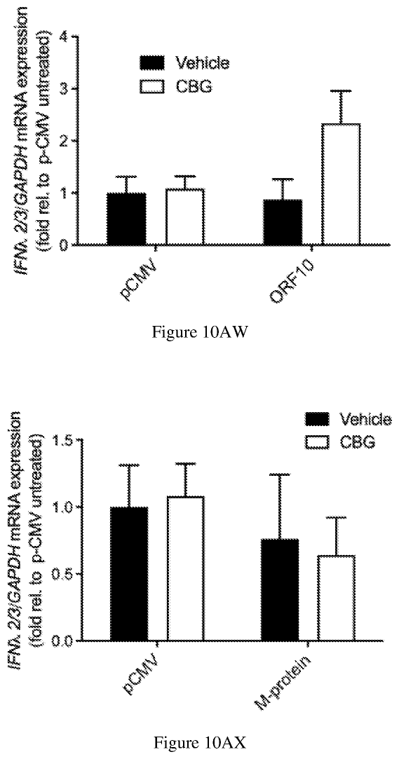

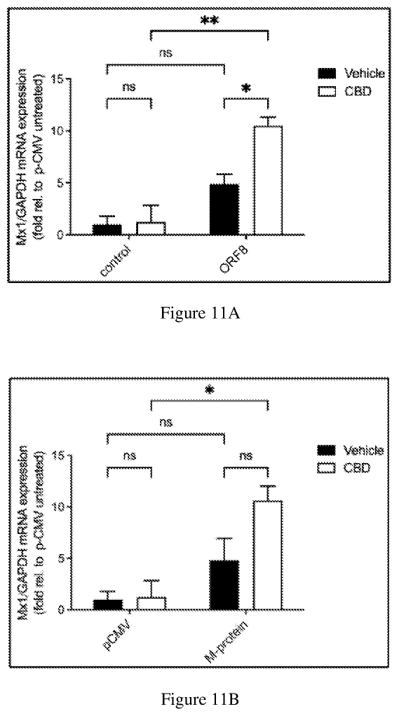

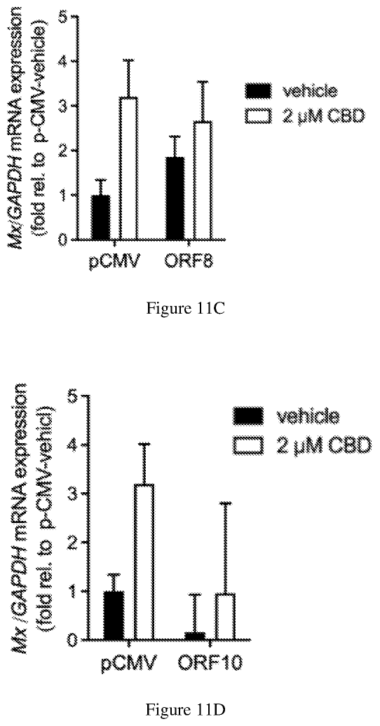

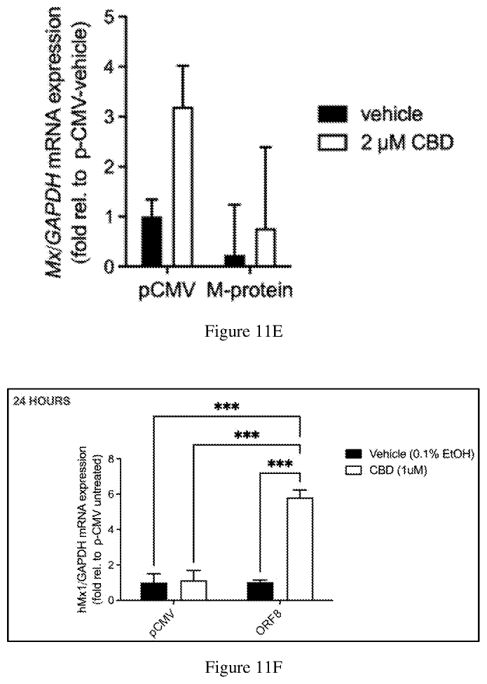

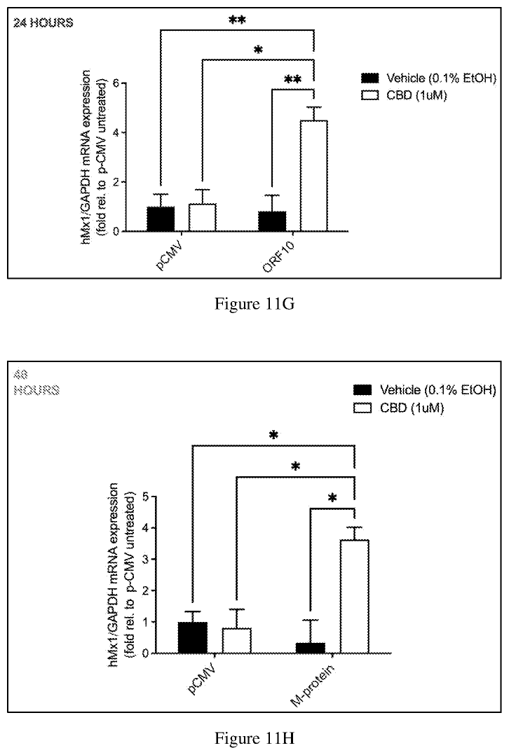

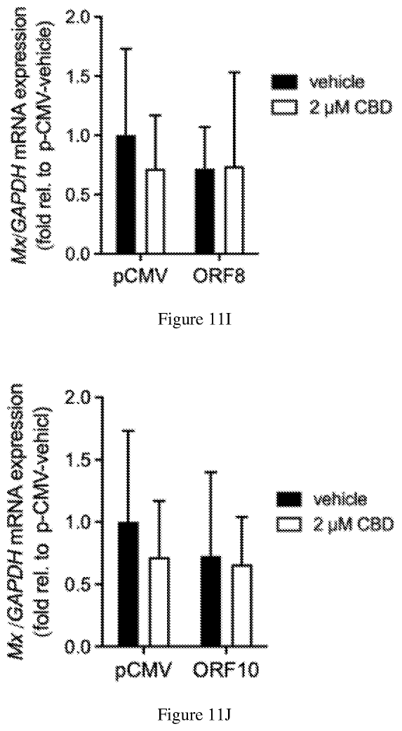

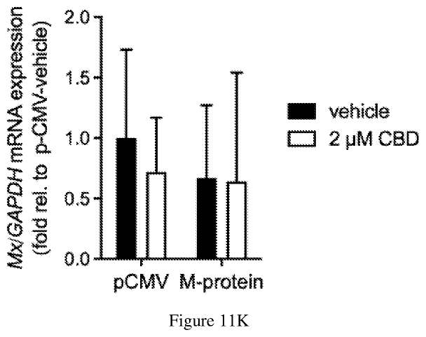

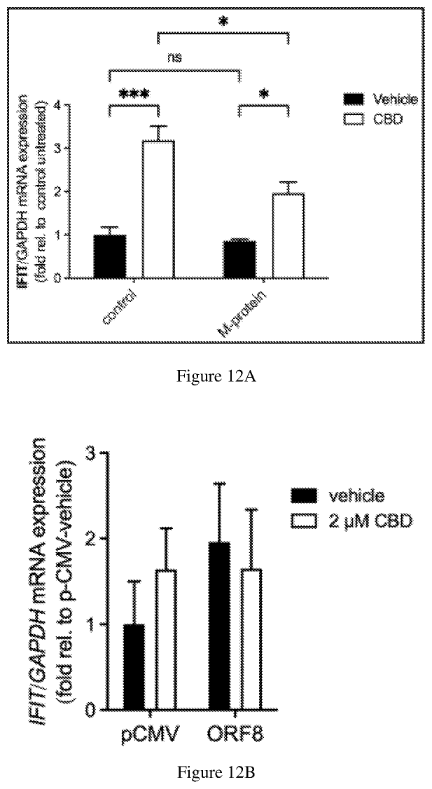

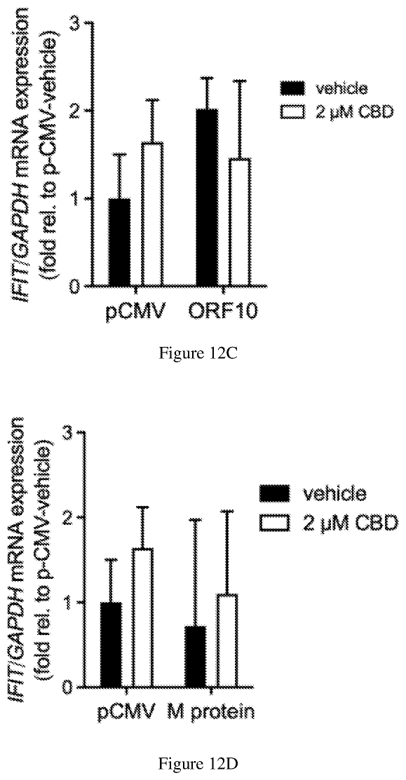

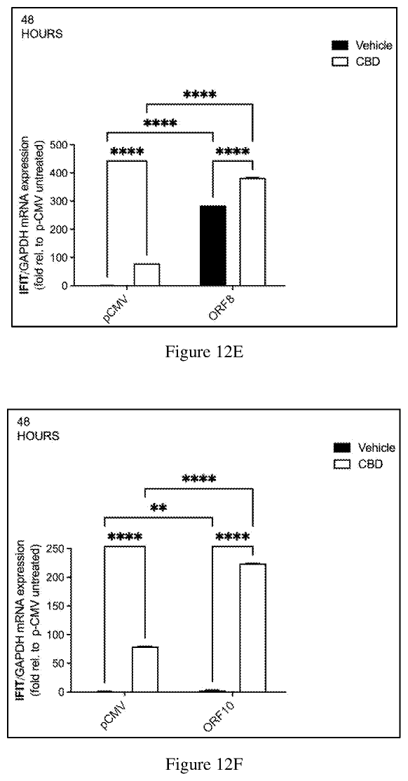

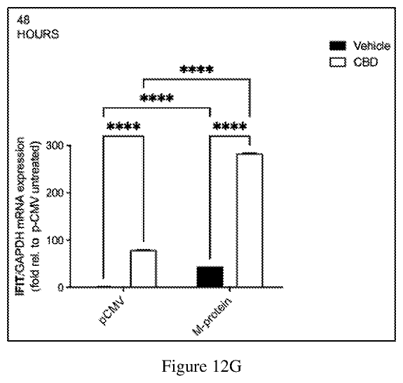

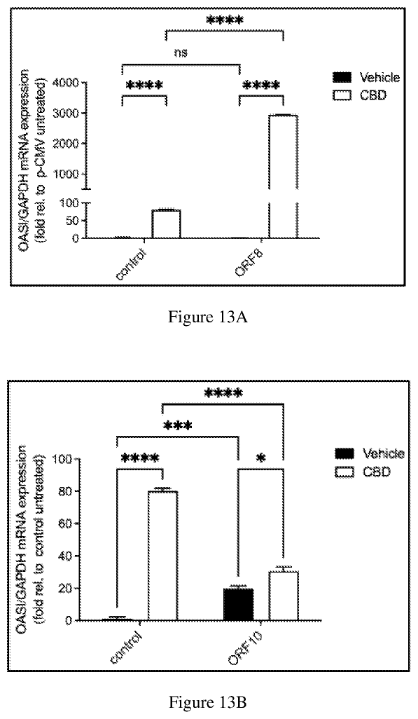

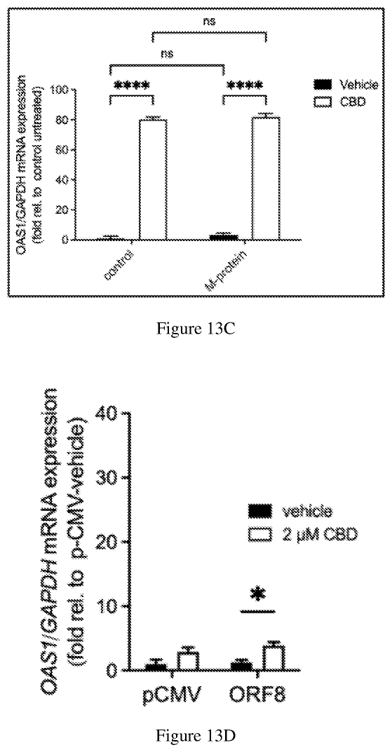

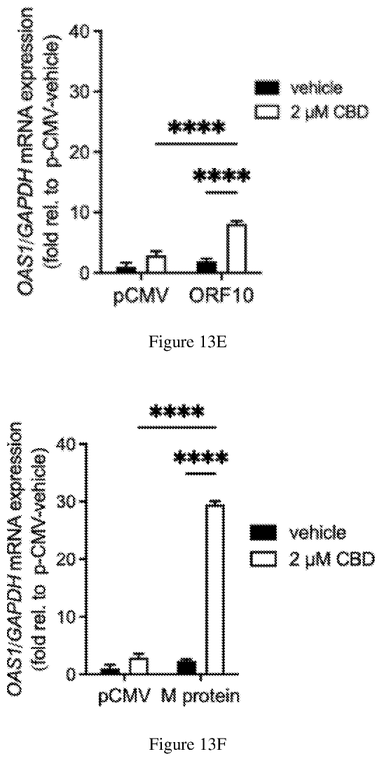

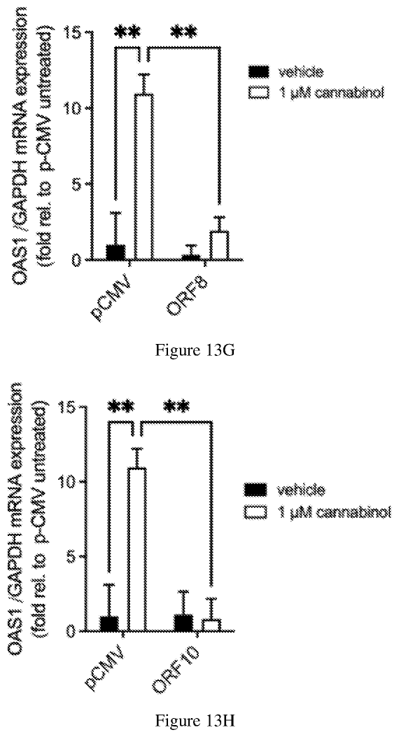

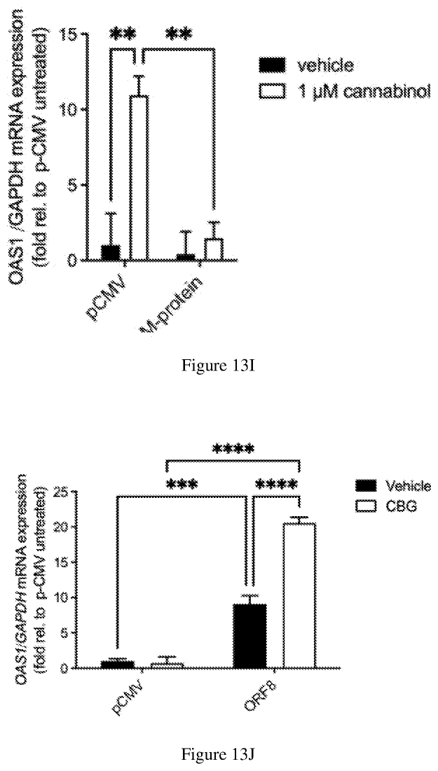

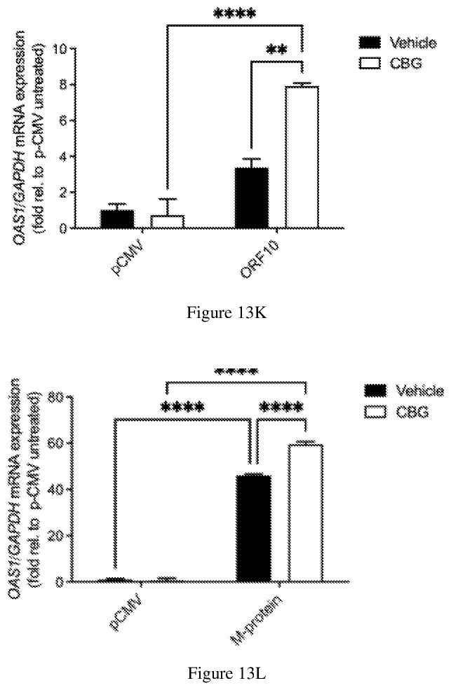

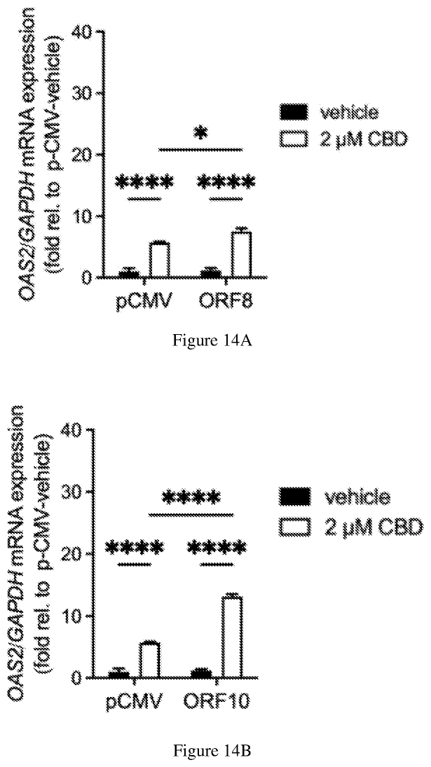

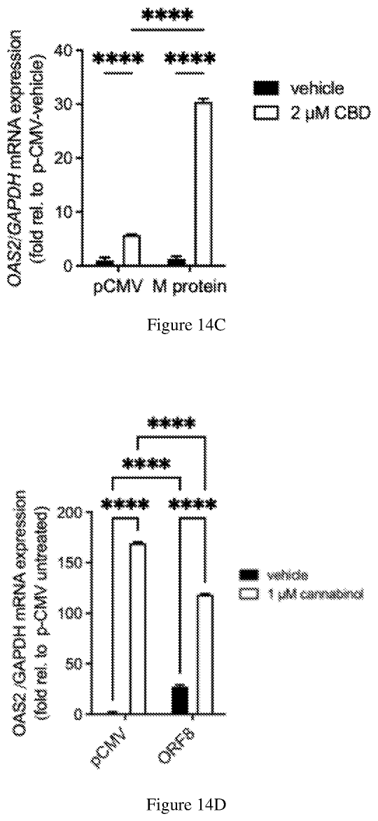

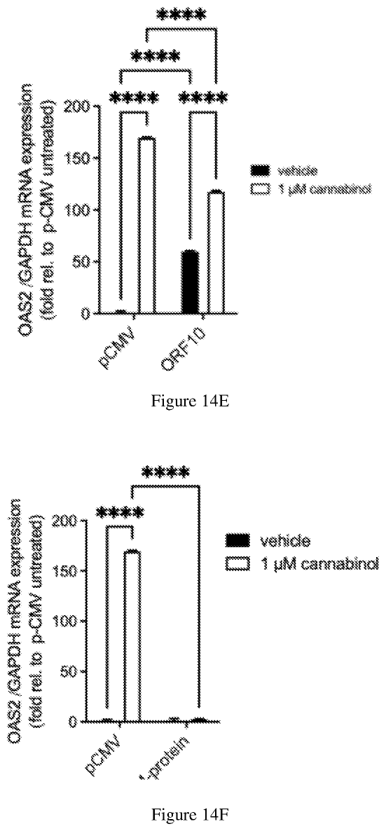

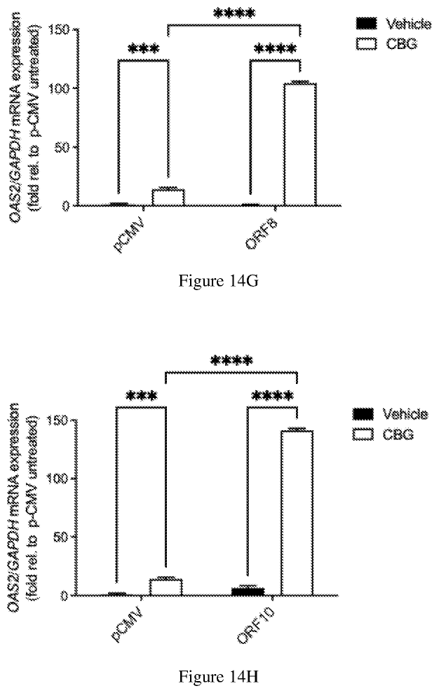

FIG. 4 provides a condition where HEK293 (human embryonic kidney) cells are transfected with plasmids expressing a vector expressing the viral Orf10 protein and further treated with Cannabidiol, cannabigerol (CBG), cannabinol, CBDA and D8. Tetrahydrocannabivarin.

Surprisingly, significant reduction in mean BrdU incorporation was observed. In cells expressing Orf10, BrdU incorporation was significantly reduced by treatment with any cannabinoid relative to untreated cells, except for delta 8-tetrahydrocannabivarin which exhibited less reduction. This could reflect a significant decrease in mean cell proliferation, or it could indicate that there are fewer cells, or a combination of these outcomes. Analysis is by 1-way ANOVA with Tukey's multiple comparison's test, where columns with different superscripts are significantly different, **P<0.01, ***P<0.001, ****P<0.0001.

In cells expressing Orf10 and treated with CBD, mean BrdU incorporation was 30.44% lower than in cells expressing Orf10 but untreated with cannabinoids.

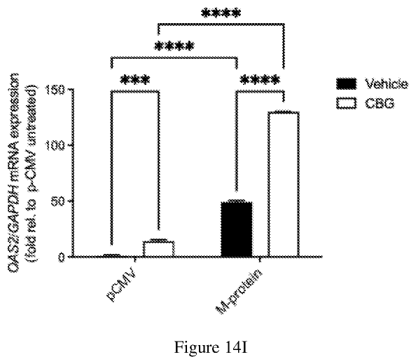

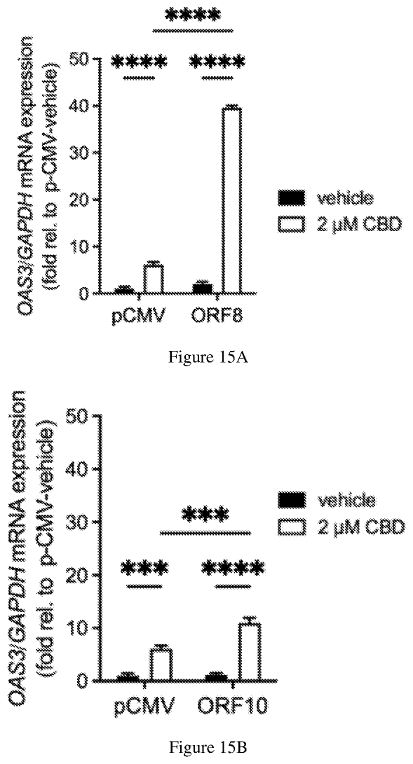

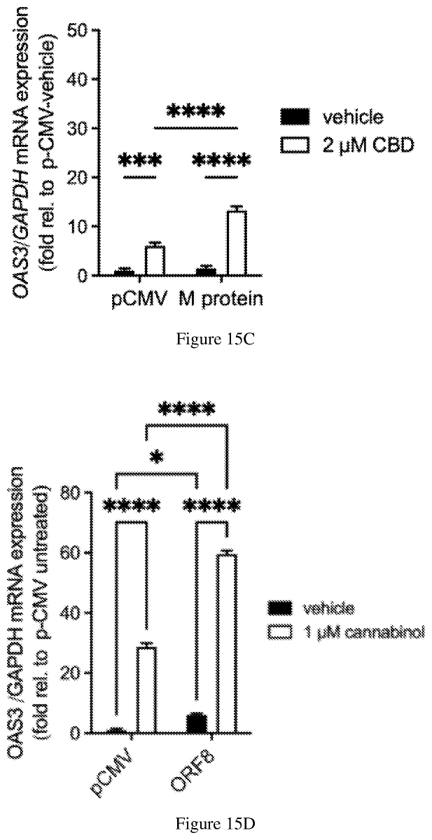

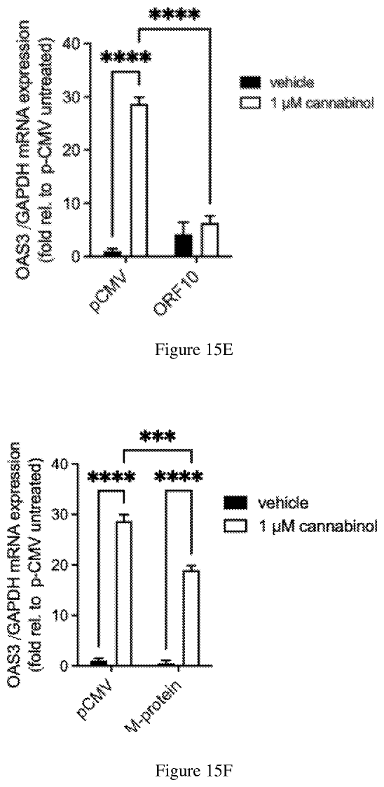

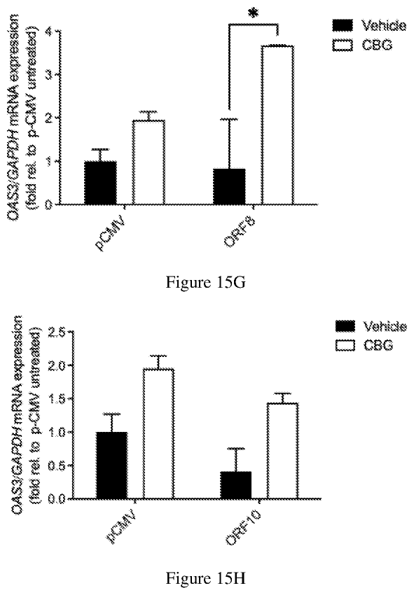

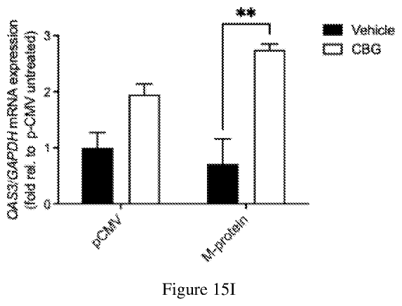

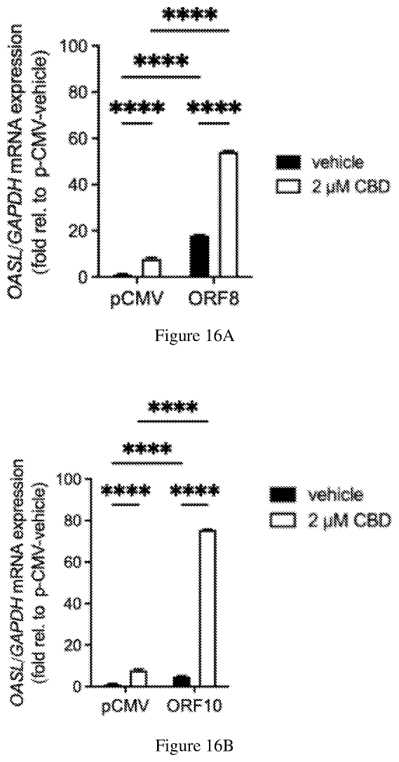

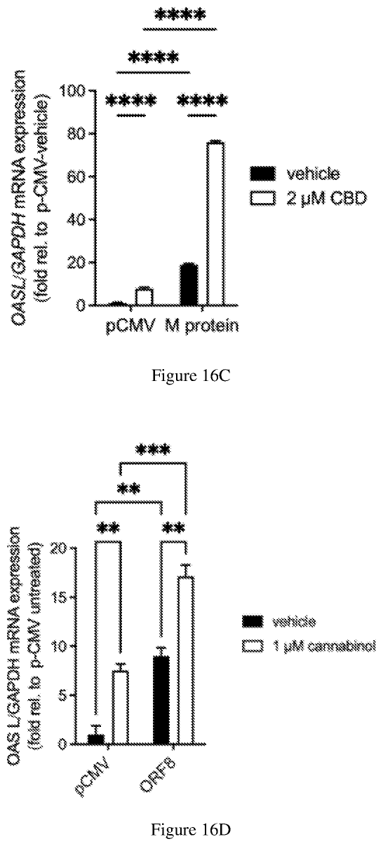

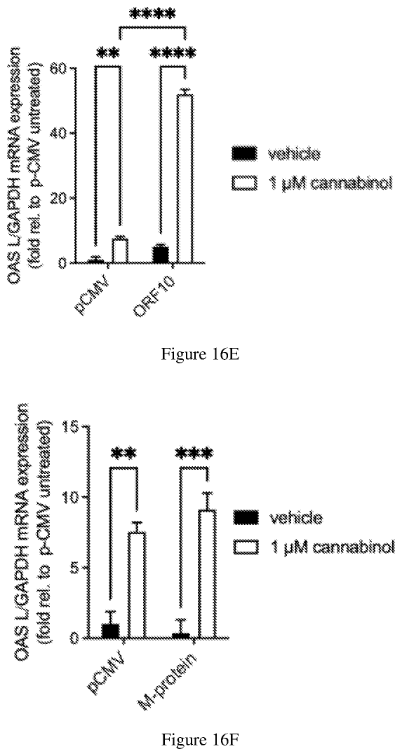

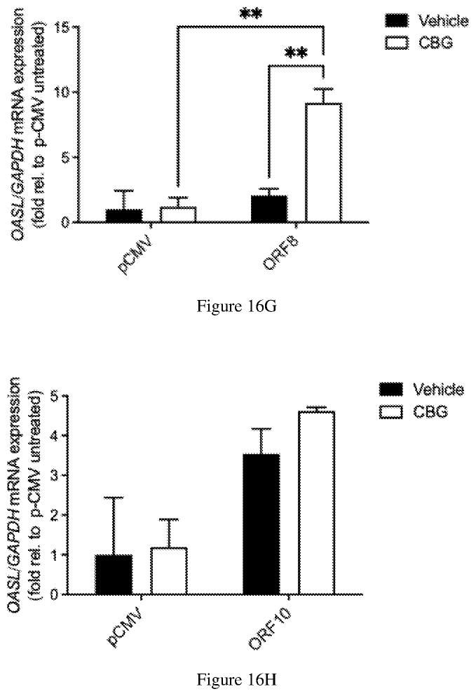

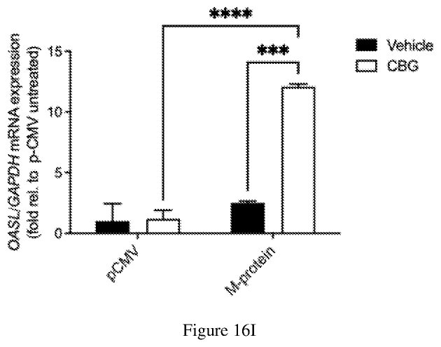

FIG. 5 provides a condition where HEK293 (human embryonic kidney) cells are transfected with plasmids expressing a vector expressing the viral M protein and further treated with Cannabidiol, cannabigerol (CBG), cannabinol, CBDA and D8-Tetrahy drocannabivarin.

Surprisingly, significant reduction in mean BrdU incorporation was observed.

In cells expressing M protein, BrdU incorporation was significantly reduced by treatment with any cannabinoid relative to untreated cells.

This could reflect a significant decrease in mean cell proliferation, or it could reflect a decrease in the number of cells in each well proliferating at the same rate, or a combination of both. Analysis is 1-way ANOVA with Bonferroni's multiple comparison's test, where columns with different superscripts are significantly different, **P<0.01.

In cells expressing M protein and treated with CBD, mean BrdU incorporation was 37.28% lower than in cells expressing M protein but untreated with cannabinoids.

FIG. 6 combines data of cell proliferation rates from all FIGS. 1-5 for ready comparison.

The combined data include cell proliferation rates of cells that are transfected with plasmids expressing an empty control vector (pCMV-3Tag-3a), ORF8, ORF10 and M protein and further treated with Cannabidiol, cannabigerol (CBG), cannabinol, CBDA and D8-Tetrahydrocannabivarin.

FIGS. 7A, 7B and 7C provide BrdU incorporation/cell proliferation, and therefore indicate the level of BrdU incorporation into nuclear DNA normalized to relative cell number in cells transfected with ORF8, ORF10 and M protein, respectively, and treated with or without Cannabidiol. These figures show that the level of BrdU incorporation per cell was not significantly different between cells transfected with control plasmid or with plasmids expressing ORF8 or ORF10 or M protein, whether treated with CBD or without (vehicle control). This indicates that the rate of HEK293 cell proliferation was not significantly altered by viral proteins or CBD, or a combination of both. It also indicated that in FIGS. 1 to 6, the decrease in BrdU incorporation was very likely due to a lower number of cells in each well, rather than a decrease in cell proliferation.

FIGS. 8A, 8B and 8C respectively provide an assay where adherent cells are stained by crystal violet and hence provide a measure of the relative cell number per well. These figures show that Cannabidiol does not significantly affect the relative number of cells per well when cells only express the control plasmid.

FIG. 8A provides relative cell number when cells are transfected with either a control plasmid or plasmid transfected with ORF8 and treated with or without Cannabidiol.

This figure shows that expression of ORF8 without Cannabidiol treatment does not reduce relative cell numbers, but relative cell numbers are decreased when cells expressing ORF8 and are treated with Cannabidiol, both in comparison to cells expressing ORF8 but treated with vehicle only, or in comparison to cells transfected with control plasmid and treated with Cannabidiol. This shows that Cannabidiol combines with this SARS-CoV-2 gene to cause a decrease in relative cell number that is only seen when the viral protein is combined with Cannabidiol.

FIG. 8B provides relative cell number when cells are transfected with either a control plasmid or plasmid transfected with ORF10 and treated with Cannabidiol.

This figure shows that expression of ORF10 without Cannabidiol treatment does not reduce relative cell numbers, but relative cell numbers are decreased when cells express ORF10 and are treated with Cannabidiol, both in comparison to cells expressing ORF10 but treated with vehicle only, or in comparison to cells transfected with control plasmid and treated with Cannabidiol. This shows that Cannabidiol combines with this SARS-CoV-2 gene to cause a decrease in relative cell number that is only seen when the viral protein is combined with Cannabidiol.

FIG. 8C provides relative cell number when cells are transfected with either a control plasmid or plasmid transfected with M protein and treated with Cannabidiol.

This figure shows that expression of M protein either with or without Cannabidiol will decrease relative cell numbers per well compared to cells transfected with the control plasmid alone and treated either with or without Cannabidiol, respectively.

However, in cells expressing M-protein, Cannabidiol treatment further enhanced the reduction in relative cell number.

FIGS. 8D, 8E, 8F and 8G respectively provides effect of ORF8, ORF10, and M protein expression, on relative number of cells, with and without Cannabidiol treatment, on HEK293 cell number.

FIG. 8D provides dose-dependent effects of Cannabidiol on the relative number of cells per well 24 h after transfection of cells with control plasmid (pCMV), or plasmids expressing ORF8, ORF10, and M protein (n=3-9).

This figure shows that expression of ORF8, ORF10, or M protein with Cannabidiol treatment reduce relative cell numbers, but relative cell numbers are not decreased when cells are transfected with only control plasmid (pCMV). There is a sharp decline in relative cell number with expression of ORF8, ORF10, or M protein with Cannabidiol treatment.

FIGS. 8E-8G provides effects of 2 μM Cannabidiol on relative cell number, n=6-12. Data are means±SEM, ****P<0.0001.

FIG. 8E provides relative cell number when cells are transfected with either a control plasmid or plasmid transfected with ORF8 and treated with or without Cannabidiol.

This figure shows that expression of ORF8 without Cannabidiol treatment does not reduce relative cell numbers, but relative cell numbers are decreased when cells express ORF8 and are treated with Cannabidiol, both in comparison to cells expressing ORF8 but treated with vehicle only, or in comparison to cells transfected with control plasmid and treated with Cannabidiol. This shows that Cannabidiol combines with this SARS-CoV-2 gene to cause a decrease in relative cell number that is only seen when the viral protein is combined with Cannabidiol.

FIG. 8F provides relative cell number when cells are transfected with either a control plasmid or plasmid transfected with ORF10 and treated with or without Cannabidiol.

This figure shows that expression of ORF10 without Cannabidiol treatment does not reduce relative cell numbers, but relative cell numbers are decreased when cells express ORF8 and are treated with Cannabidiol, both in comparison to cells expressing ORF10 but treated with vehicle only, or in comparison to cells transfected with control plasmid and treated with Cannabidiol. This shows that Cannabidiol combines with this SARS-CoV-2 gene to cause a decrease in relative cell number that is only seen when the viral protein is combined with Cannabidiol.

FIG. 8G provides relative cell number when cells are transfected with either a control plasmid or plasmid transfected with M protein and treated with or without Cannabidiol.

This figure shows that expression of M protein without Cannabidiol treatment does not reduce relative cell numbers, but relative cell numbers are decreased when cells express ORF8 and are treated with Cannabidiol, both in comparison to cells expressing M protein but treated with vehicle only, or in comparison to cells transfected with control plasmid and treated with Cannabidiol. This shows that Cannabidiol combines with this SARS-CoV-2 gene to cause a decrease in relative cell number that is only seen when the viral protein is combined with Cannabidiol.

FIGS. 9A and 9B provide respectively an early and late apoptosis data of HEK293 (human embryonic kidney) cells transfected with i) control plasmid expressing control vector and ii) plasmid expressing viral protein ORF8; and then treated with Cannabidiol. Cannabidiol treated cells which are transfected with control plasmid do not show any significant increase in early as well as late apoptosis but Cannabidiol treated cells which are transfected with plasmid expressing viral protein ORF8 have exhibited significant increases in early apoptosis and late apoptosis, both relative to ORF8-expressing cells treated only with vehicle control, and relative to control vector-expressing cells treated with Cannabidiol, indicating that Cannabidiol augments the cellular pro-apoptotic anti-viral response to ORF8, and this is specific to cells expressing ORF8.

FIGS. 9C and 9D respectively provide early apoptosis and late apoptosis data in cells transfected with a control plasmid or viral plasmid expressing ORF10 and treated with Cannabidiol. Cannabidiol induces apoptosis in cells transfected with ORF10 and treated with Cannabidiol to a significantly greater extent than in cells treated with Cannabidiol but expressing only control plasmid, indicating a specific ability of Cannabidiol to augment apoptosis when present in combination with the SARS-CoV-2 ORF10 protein, but not when anon-viral plasmid is present.

FIGS. 9E and 9F respectively provide early apoptosis and late apoptosis data in cells transfected with a control plasmid or viral plasmid expressing M protein and treated with Cannabidiol. Cells transfected with M protein and treated with Cannabidiol significantly increased both early and late apoptosis compared to cells treated under the same conditions but transfected only with control plasmid. Cells transfected with M-protein and treated with Cannabidiol had significantly elevated early and late apoptosis also relative to cells expressing M protein but treated only with vehicle.

FIGS. 9G and 9H respectively provides effect of ORF8, ORF10, or M protein expression, with and without CBD, on measures of early and late apoptosis. Dose-dependent effects of Cannabidiol on early and late apoptosis in cells transfected with control plasmid (pCMV), or plasmids expressing ORF8, ORF10 and M protein (n=3-9) are shown. Cannabidiol treated cells which are transfected with control plasmid do not show any significant increase in early as well as late apoptosis, but Cannabidiol treated cells which are transfected with plasmid expressing the viral proteins ORF8, ORF10 and M protein have exhibited significant increases in early apoptosis and late apoptosis.

FIG. 9I provide an early apoptosis data of HEK293 (human embryonic kidney) cells transfected with i) control plasmid expressing control vector and ii) plasmid expressing viral protein ORF8; and then treated with 2 μM Cannabidiol.

Cannabidiol treated cells which are transfected with control plasmid do not show any significant increase in early apoptosis, but Cannabidiol treated cells which are transfected with plasmid expressing viral protein ORF8 have exhibited significant increases in early apoptosis, both relative to ORF8-expressing cells treated only with vehicle control, and relative to control vector-expressing cells treated with Cannabidiol.

FIG. 9J provide an early apoptosis data of HEK293 (human embryonic kidney) cells transfected with i) control plasmid expressing control vector and ii) plasmid expressing viral protein ORF10; and then treated with 2 μM Cannabidiol.

Cannabidiol treated cells which are transfected with control plasmid do not show any significant increase in early apoptosis, but Cannabidiol treated cells which are transfected with plasmid expressing viral protein ORF10 have exhibited significant increases in early apoptosis, both relative to ORF10-expressing cells treated only with vehicle control, and relative to control vector-expressing cells treated with Cannabidiol.

FIG. 9K provide early apoptosis data of HEK293 (human embryonic kidney) cells transfected with i) control plasmid expressing control vector and ii) plasmid expressing viral M Protein; and then treated with 2 μM Cannabidiol. Cannabidiol treated cells which are transfected with control plasmid do not show any significant increase in early apoptosis, but Cannabidiol treated cells which are transfected with plasmid expressing viral M Protein have exhibited significant increases in early apoptosis, both relative to viral M Protein-expressing cells treated only with vehicle control, and relative to control vector-expressing cells treated with Cannabidiol.

FIG. 9L provide a late apoptosis data of HEK293 (human embryonic kidney) cells transfected with i) control plasmid expressing control vector and ii) plasmid expressing viral protein ORF8; and then treated with 2 μM Cannabidiol. Cannabidiol treated cells which are transfected with control plasmid do not show any significant increase in late apoptosis, but Cannabidiol treated cells which are transfected with plasmid expressing viral protein ORF8 have exhibited significant increases in late apoptosis, both relative to ORF8-expressing cells treated only with vehicle control, and relative to control vector-expressing cells treated with Cannabidiol.

FIG. 9M provide a late apoptosis data of HEK293 (human embryonic kidney) cells transfected with i) control plasmid expressing control vector and ii) plasmid expressing viral protein ORF10; and then treated with 2 μM Cannabidiol. Cannabidiol treated cells which are transfected with control plasmid do not show any significant increase in late apoptosis, but Cannabidiol treated cells which are transfected with plasmid expressing viral protein ORF10 have exhibited significant increases in late apoptosis, both relative to ORF10-expressing cells treated only with vehicle control, and relative to control vector-expressing cells treated with Cannabidiol.

FIG. 9N provide a late apoptosis data of HEK293 (human embryonic kidney) cells transfected with i) control plasmid expressing control vector and ii) plasmid expressing viral M Protein; and then treated with 2 μM Cannabidiol. Cannabidiol treated cells which are transfected with control plasmid do not show any significant increase in late apoptosis, but Cannabidiol treated cells which are transfected with plasmid expressing viral M Protein have exhibited significant increases in late apoptosis, both relative to viral M Protein-expressing cells treated only with vehicle control, and relative to control vector-expressing cells treated with Cannabidiol.

FIGS. 9O to 9P provide effect of ORF8, ORF10, or M protein expression, with and without cannabinol on measures of early and late apoptosis. Dose-dependent effects of cannabinol on early and late apoptosis in cells transfected with control plasmid (pCMV), or plasmids expressing ORF8, ORF10, or M protein (n=3-6) are shown. Cannabidiol treated cells which are transfected with control plasmid show some significant increases in early as well as late apoptosis and cannabinol treated cells which are transfected with plasmid expressing viral protein ORF8, ORF10 and M protein have also exhibited significant increases in early apoptosis and late apoptosis.

FIGS. 9Q to 9S provides effects of 1 μM cannabinol on measures of early apoptosis. Data are means±SEM, *P<0.05, **P<0.01,***P<0.01.

FIG. 9Q provide an early apoptosis data of HEK293 (human embryonic kidney) cells transfected with i) control plasmid expressing control vector and ii) plasmid expressing viral protein ORF8; and then treated with 1 μM cannabinol. Cannabinol treated cells which are transfected with control plasmid show a significant increase in early apoptosis, and cannabinol treated cells which are transfected with plasmid expressing viral protein ORF8 have also exhibited significant increases in early apoptosis relative to ORF8-expressing cells treated only with vehicle control.

FIG. 9R provide an early apoptosis data of HEK293 (human embryonic kidney) cells transfected with i) control plasmid expressing control vector and ii) plasmid expressing viral protein ORF10; and then treated with 1 μM cannabinol. Cannabinol treated cells which are transfected with control plasmid show a significant increase in early apoptosis, and cannabinol treated cells which are transfected with plasmid expressing viral protein ORF10 also exhibited significant increases in early apoptosis, relative to ORF10-expressing cells treated only with vehicle control.

FIG. 9S provides early apoptosis data of HEK293 (human embryonic kidney) cells transfected with i) control plasmid expressing control vector and ii) plasmid expressing viral M Protein; and then treated with 1 μM cannabinol. Cannabinol treated cells which are transfected with control plasmid show a significant increase in early apoptosis, and cannabinol treated cells which are transfected with plasmid expressing viral M Protein also exhibited significant increases in early apoptosis, relative to viral M Protein-expressing cells treated only with vehicle control.

FIGS. 9T to 9V provide effects of 1 μM cannabinol on measures of late apoptosis. Data are means±SEM, *P<0.05, **P<0.01, ***P<0.01.

FIG. 9T provides late apoptosis data of HEK293 (human embryonic kidney) cells transfected with i) control plasmid expressing control vector and ii) plasmid expressing viral protein ORF8; and then treated with 1 μM cannabinol. Cannabinol treated cells which are transfected with control plasmid show a significant increase in late apoptosis, and cannabinol treated cells which are transfected with plasmid expressing viral protein ORF8 also exhibited significant increases in late apoptosis, relative to ORF8-expressing cells treated only with vehicle control.

FIG. 9U provides late apoptosis data of HEK293 (human embryonic kidney) cells transfected with i) control plasmid expressing control vector and ii) plasmid expressing viral protein ORF10; and then treated with 1 μM Cannabinol. Cannabinol treated cells which are transfected with control plasmid show a significant increase in late apoptosis, and Cannabinol treated cells which are transfected with plasmid expressing viral protein ORF10 also have exhibited significant increases in late apoptosis, both relative to ORF10-expressing cells treated only with vehicle control, and relative to control vector-expressing cells treated with Cannabinol.

FIG. 9V provides late apoptosis data of HEK293 (human embryonic kidney) cells transfected with i) control plasmid expressing control vector and ii) plasmid expressing viral M Protein; and then treated with 1 μM cannabinol. Cannabinol treated cells which are transfected with control plasmid show a significant increase in late apoptosis and cannabinol treated cells which are transfected with plasmid expressing viral M Protein have significant increases in late apoptosis, relative to viral M Protein-expressing cells treated only with vehicle control.

FIGS. 9W and 9X provide effect of ORF8, ORF10, or M protein expression, with and without cannabinol, on measures of early and late apoptosis. Dose-dependent effects of cannabinol on early and late apoptosis in cells transfected with control plasmid (pCMV), or plasmids expressing ORF8, ORF10, or M protein (n=3-6) are shown. Cannabinol treated cells which are transfected with control plasmid show some significant increases in early as well as late apoptosis, and cannabinol treated cells which are transfected with plasmid expressing viral protein ORF8, ORF10 and M protein have exhibited significant increases in early apoptosis and late apoptosis.

FIGS. 9Y to 9AA provide effects of 2 μM cannabinol on measures of early apoptosis. Data are means±SEM, *P<0.05, **P<0.01,***P<0.01.

FIG. 9Y provide an early apoptosis data of HEK293 (human embryonic kidney) cells transfected with i) control plasmid expressing control vector and ii) plasmid expressing viral protein ORF8; and then treated with 2 μM cannabinol. Cannabinol treated cells which are transfected with control plasmid show a significant increase in early apoptosis, and cannabinol treated cells which are transfected with plasmid expressing viral protein ORF8 have exhibited significant increases in early apoptosis, both relative to ORF8-expressing cells treated only with vehicle control, and relative to control vector-expressing cells treated with cannabinol.

FIG. 9Z provides early apoptosis data of HEK293 (human embryonic kidney) cells transfected with i) control plasmid expressing control vector and ii) plasmid expressing viral protein ORF10; and then treated with 2 μM cannabinol. Cannabinol treated cells which are transfected with control plasmid show a significant increase in early apoptosis, and cannabinol treated cells which are transfected with plasmid expressing viral protein ORF10 exhibited significant increases in early apoptosis, relative to ORF10-expressing cells treated only with vehicle control.

FIG. 9AA provides early apoptosis data of HEK293 (human embryonic kidney) cells transfected with i) control plasmid expressing control vector and ii) plasmid expressing viral M Protein; and then treated with 2 μM cannabinol. Cannabinol treated cells which are transfected with control plasmid show a significant increase in early apoptosis, and cannabinol treated cells which are transfected with plasmid expressing viral M Protein exhibited significant increases in early apoptosis, both relative to viral M Protein-expressing cells treated only with vehicle control, and relative to control vector-expressing cells treated with cannabinol.

FIGS. 9AB to 9AD provides effects of 2 μM cannabinol on measures of late apoptosis. Data are means±SEM, *P<0.05, **P<0.01, ***P<0.01.

FIG. 9AB provide a late apoptosis data of HEK293 (human embryonic kidney) cells transfected with i) control plasmid expressing control vector and ii) plasmid expressing viral protein ORF8; and then treated with 2 μM cannabinol. Cannabinol treated cells which are transfected with control plasmid show a significant increase in late apoptosis, and cannabinol treated cells which are transfected with plasmid expressing viral protein ORF8 also exhibited significant increases in late apoptosis, relative to ORF8-expressing cells treated only with vehicle control, but not relative to control vector-expressing cells treated with cannabinol.

FIG. 9AC provide a late apoptosis data of HEK293 (human embryonic kidney) cells transfected with i) control plasmid expressing control vector and ii) plasmid expressing viral protein ORF10; and then treated with 2 μM cannabinol. Cannabinol treated cells which are transfected with control plasmid show a significant increase in late apoptosis, and cannabinol treated cells which are transfected with plasmid expressing viral protein ORF10 also exhibited significant increases in late apoptosis, relative to ORF10-expressing cells treated only with vehicle control, but not relative to control vector-expressing cells treated with cannabinol.

FIG. 9AD provide a late apoptosis data of HEK293 (human embryonic kidney) cells transfected with i) control plasmid expressing control vector and ii) plasmid expressing viral M Protein; and then treated with 2 μM cannabinol. Cannabinol treated cells which are transfected with control plasmid show a significant increase in late apoptosis, and cannabinol treated cells which are transfected with plasmid expressing viral M Protein also have exhibited significant increases in late apoptosis, both relative to viral M Protein-expressing cells treated only with vehicle control, and relative to control vector-expressing cells treated with cannabinol.

FIGS. 9AE to 9AJ provides effect of ORF8, ORF10, or M protein expression, with and without cannabidiolic acid (CBDA), on measures of early and late apoptosis. Effects of 1 μM CBDA on measures of early and late apoptosis (n=3) are shown. Data are means±SEM, *P<0.05, **P<0.01, ***P<0.01.

FIG. 9AE provide an early apoptosis data of HEK293 (human embryonic kidney) cells transfected with i) control plasmid expressing control vector and ii) plasmid expressing viral protein ORF8; and then treated with 1 μM CBDA. CBDA treated cells which are transfected with control plasmid do not show any significant increase in early apoptosis, but CBDA treated cells which are transfected with plasmid expressing viral protein ORF8 exhibited significant increases in early apoptosis, relative to control vector-expressing cells treated with CBDA.

FIG. 9AF provide an early apoptosis data of HEK293 (human embryonic kidney) cells transfected with i) control plasmid expressing control vector and ii) plasmid expressing viral protein ORF10; and then treated with 1 μM CBDA. CBDA treated cells, which are transfected with control plasmid, do not show any significant increase in early apoptosis but CBDA treated cells which are transfected with plasmid expressing viral protein ORF10 have exhibited significant increases in early apoptosis relative to control vector-expressing cells treated with CBDA.

FIG. 9AG provide an early apoptosis data of HEK293 (human embryonic kidney) cells transfected with i) control plasmid expressing control vector and ii) plasmid expressing viral M Protein; and then treated with 1 μM CBDA. CBDA treated cells which are transfected with control plasmid do not show any significant increase in early apoptosis, but CBDA treated cells which are transfected with plasmid expressing viral M Protein have exhibited significant increases in early apoptosis relative to control vector-expressing cells treated with CBDA.

FIG. 9AH provide a late apoptosis data of HEK293 (human embryonic kidney) cells transfected with i) control plasmid expressing control vector and ii) plasmid expressing viral protein ORF8; and then treated with 1 μM CBDA. CBDA treated cells which are transfected with control plasmid do not show any significant increase in late apoptosis and neither do CBDA treated cells which are transfected with plasmid expressing viral protein ORF8, either relative to ORF8-expressing cells treated only with vehicle control, or relative to control vector-expressing cells treated with CBDA.

FIG. 9AI provides a late apoptosis data of HEK293 (human embryonic kidney) cells transfected with i) control plasmid expressing control vector and ii) plasmid expressing viral protein ORF10; and then treated with 1 μM CBDA. CBDA treated cells which are transfected with control plasmid do not show any significant increase in late apoptosis, but CBDA treated cells which are transfected with plasmid expressing viral protein ORF10 have exhibited a significant increase in late apoptosis relative to control vector-expressing cells treated with CBDA.

FIG. 9AJ provide a late apoptosis data of HEK293 (human embryonic kidney) cells transfected with i) control plasmid expressing control vector and ii) plasmid expressing viral M Protein; and then treated with 1 μM CBDA. CBDA treated cells which are transfected with control plasmid do not show any significant increase in late apoptosis, but CBDA treated cells which are transfected with plasmid expressing viral M Protein have exhibited significant increases in late apoptosis relative to control vector-expressing cells treated with CBDA.

FIGS. 9AK to 9AL provides effect of ORF8, ORF10, or M protein expression, with and without Cannabigerol (CBG), on measures of early and late apoptosis. Dose-dependent effects of CBG on early and late apoptosis in cells transfected with control plasmid (pCMV), or plasmids expressing ORF8, ORF10, or M protein (n=3-6) are shown. CBG treated cells which are transfected with control plasmid show some significant increase in early as well as late apoptosis, and CBG treated cells which are transfected with plasmid expressing viral protein ORF8, ORF10 and M protein have also exhibited significant increases in early apoptosis and late apoptosis.

FIGS. 9AM to 9AO provides effects of 1 μM CBG on measures of early apoptosis. Data are means±SEM, *P<0.05, **P<0.01, ***P<0.01.

FIG. 9AM provide an early apoptosis data of HEK293 (human embryonic kidney) cells transfected with i) control plasmid expressing control vector and ii) plasmid expressing viral protein ORF8; and then treated with 1 μM CBG. CBG treated cells which are transfected with control plasmid show a significant increase in early apoptosis and CBG treated cells which are transfected with plasmid expressing viral protein ORF8 also have exhibited a significant increases in early apoptosis relative to ORF8-expressing cells treated only with vehicle control.

FIG. 9AN provides early apoptosis data of HEK293 (human embryonic kidney) cells transfected with i) control plasmid expressing control vector and ii) plasmid expressing viral protein ORF10; and then treated with 1 μM CBG. CBG treated cells which are transfected with control plasmid show significantly increased early apoptosis and CBG treated cells which are transfected with plasmid expressing viral protein ORF10 have exhibited significant increases in early apoptosis relative to ORF10-expressing cells treated only with vehicle control.

FIG. 9AO provides early apoptosis data of HEK293 (human embryonic kidney) cells transfected with i) control plasmid expressing control vector and ii) plasmid expressing viral M Protein; and then treated with 1 μM CBG. CBG treated cells which are transfected with control plasmid show a significant increase in early apoptosis and CBG treated cells which are transfected with plasmid expressing viral M Protein have exhibited significant increases in early apoptosis relative to viral M Protein-expressing cells treated only with vehicle control.

FIGS. 9AP to 9AR provides effects of 1 μM CBG on measures of late apoptosis. Data are means±SEM, *P<0.05, **P<0.01, ***P<0.01.

FIG. 9AP provide a late apoptosis data of HEK293 (human embryonic kidney) cells transfected with i) control plasmid expressing control vector and ii) plasmid expressing viral protein ORF8; and then treated with 1 μM CBG. CBG treated cells which are transfected with control plasmid do not show any significant increase in late apoptosis, but CBG treated cells which are transfected with plasmid expressing viral protein ORF8 exhibited a significant increase in late apoptosis relative to ORF8-expressing cells treated only with vehicle control.

FIG. 9AQ provide a late apoptosis data of HEK293 (human embryonic kidney) cells transfected with i) control plasmid expressing control vector and ii) plasmid expressing viral protein ORF10; and then treated with 1 μM CBG. CBG treated cells which are transfected with control plasmid show a significant increase in late apoptosis and CBG treated cells which are transfected with plasmid expressing viral protein ORF10 exhibited a significant increases in late apoptosis relative to ORF10-expressing cells treated only with vehicle control.

FIG. 9AR provides late apoptosis data of HEK293 (human embryonic kidney) cells transfected with i) control plasmid expressing control vector and ii) plasmid expressing viral M Protein; and then treated with 1 μM CBG. CBG treated cells which are transfected with control plasmid do not show any significant increase in late apoptosis, but CBG treated cells which are transfected with plasmid expressing viral M Protein exhibited a significant increases in late apoptosis relative to viral M Protein-expressing cells treated only with vehicle control.

FIGS. 9AS to 9AT provides effect of ORF8, ORF10, or M protein expression, with and without Cannabigerol (CBG), on measures of early and late apoptosis. Dose-dependent effects of CBG on early and late apoptosis in cells transfected with control plasmid (pCMV), or plasmids expressing ORF8, ORF10, or M protein (n=3-6) are shown. CBG treated cells which are transfected with control plasmid show some significant increases in early as well as late apoptosis and CBG treated cells which are transfected with plasmid expressing viral protein ORF8, ORF10 and M protein have exhibited significant increases in early apoptosis and late apoptosis.

FIGS. 9AU to 9AW provides effects of 2 μM CBG on measures of early apoptosis. Data are means±SEM, *P<0.05, **P<0.01, ***P<0.01.

FIG. 9AU provide an early apoptosis data of HEK293 (human embryonic kidney) cells transfected with i) control plasmid expressing control vector and ii) plasmid expressing viral protein ORF8; and then treated with 2 μM CBG. CBG treated cells which are transfected with control plasmid show a significant increase in early apoptosis and CBG treated cells which are transfected with plasmid expressing viral protein ORF8 exhibited a significant increase in early apoptosis, both relative to ORF8-expressing cells treated only with vehicle control, and relative to control vector-expressing cells treated with CBG.

FIG. 9AV provide an early apoptosis data of HEK293 (human embryonic kidney) cells transfected with i) control plasmid expressing control vector and ii) plasmid expressing viral protein ORF10; and then treated with 2 μM CBG. CBG treated cells which are transfected with control plasmid show a significant increase in early apoptosis and CBG treated cells which are transfected with plasmid expressing viral protein ORF10 have exhibited significant increases in early apoptosis relative to ORF10-expressing cells treated only with vehicle control.

FIG. 9AW provide an early apoptosis data of HEK293 (human embryonic kidney) cells transfected with i) control plasmid expressing control vector and ii) plasmid expressing viral M Protein; and then treated with 2 μM CBG. CBG treated cells which are transfected with control plasmid show a significant increase in early apoptosis and CBG treated cells which are transfected with plasmid expressing viral M Protein have exhibited significant increases in early apoptosis, both relative to viral M Protein-expressing cells treated only with vehicle control, and relative to control vector-expressing cells treated with CBG.

FIGS. 9AX to 9AZ provides effects of 2 μM CBG on measures of late apoptosis. Data are means±SEM, *P<0.05, **P<0.01, ***P<0.01.

FIG. 9AX provide a late apoptosis data of HEK293 (human embryonic kidney) cells transfected with i) control plasmid expressing control vector and ii) plasmid expressing viral protein ORF8; and then treated with 2 μM CBG. CBG treated cells which are transfected with control plasmid show a significant increase in late apoptosis and CBG treated cells which are transfected with plasmid expressing viral protein ORF8 have exhibited significant increases in late apoptosis relative to ORF8-expressing cells treated only with vehicle control.

FIG. 9AY provides late apoptosis data of HEK293 (human embryonic kidney) cells transfected with i) control plasmid expressing control vector and ii) plasmid expressing viral protein ORF10; and then treated with 2 μM CBG. CBG treated cells which are transfected with control plasmid show a significant increase in late apoptosis, but CBG treated cells which are transfected with plasmid expressing viral protein ORF10 exhibited a significant increase in late apoptosis relative to ORF10-expressing cells treated only with vehicle control.

FIG. 9AZ provide a late apoptosis data of HEK293 (human embryonic kidney) cells transfected with i) control plasmid expressing control vector and ii) plasmid expressing viral M Protein; and then treated with 2 μM CBG. CBG treated cells which are transfected with control plasmid show a significant increase in late apoptosis and CBG treated cells which are transfected with plasmid expressing viral M Protein have exhibited significant increases in late apoptosis, both relative to viral M Protein-expressing cells treated only with vehicle control, and relative to control vector-expressing cells treated with CBG.

FIGS. 9BA to 9BE provide effect of ORF8, ORF10 and M protein expression, with and without delta 8-tetrahydrocannabivarin (d8-THCV), on measures of early and late apoptosis.

FIGS. 9BA to 9BC provide effects of 1 μM d8-THCV on measures of early apoptosis (n=3). Data are means±SEM, *P<0.05, **P<0.01.

FIG. 9BA provides early apoptosis data of HEK293 (human embryonic kidney) cells transfected with i) control plasmid expressing control vector and ii) plasmid expressing viral protein ORF8; and then treated with 1 μM d8-THCV. d8-THCV treated cells which are transfected with control plasmid do not show any significant increase in early apoptosis, but d8-THCV treated cells which are transfected with plasmid expressing viral protein ORF8 have exhibited a significant increase in early apoptosis and relative to control vector-expressing cells treated with d8-THCV.

FIG. 9BB provides early apoptosis data of HEK293 (human embryonic kidney) cells transfected with i) control plasmid expressing control vector and ii) plasmid expressing viral protein ORF10; and then treated with 1 μM d8-THCV. d8-THCV treated cells which are transfected with control plasmid do not show any significant increase in early apoptosis, but d8-THCV treated cells which are transfected with plasmid expressing viral protein ORF10 have exhibited significant increases in early apoptosis, both relative to ORF10-expressing cells treated only with vehicle control, and relative to control vector-expressing cells treated with d8-THCV.

FIG. 9BC provide an early apoptosis data of HEK293 (human embryonic kidney) cells transfected with i) control plasmid expressing control vector and ii) plasmid expressing viral M Protein; and then treated with 1 μM d8-THCV. d8-THCV treated cells which are transfected with control plasmid do not show any significant increase in early apoptosis, but d8-THCV treated cells which are transfected with plasmid expressing viral M Protein have exhibited significant increases in early apoptosis, both relative to viral M Protein-expressing cells treated only with vehicle control, and relative to control vector-expressing cells treated with d8-THCV.

FIGS. 9BD to 9BF provides effects of 1 μM d8-THCV on measures of late apoptosis. Data are means±SEM, *P<0.05, **P<0.01, ***P<0.01.

FIG. 9BD provide a late apoptosis data of HEK293 (human embryonic kidney) cells transfected with i) control plasmid expressing control vector and ii) plasmid expressing viral protein ORF8; and then treated with 1 μM d8-THCV. d8-THCV treated cells which are transfected with control plasmid show a significant increase in late apoptosis and d8-THCV treated cells which are transfected with plasmid expressing viral protein ORF8 also have exhibited a significant increase in late apoptosis relative to ORF8-expressing cells treated only with vehicle control.

FIG. 9BE provide a late apoptosis data of HEK293 (human embryonic kidney) cells transfected with i) control plasmid expressing control vector and ii) plasmid expressing viral protein ORF10; and then treated with 1 μM d8-THCV. d8-THCV treated cells which are transfected with control plasmid show a significant increase in late apoptosis, but d8-THCV treated cells which are transfected with plasmid expressing viral protein ORF10 do not have significant increases in late apoptosis, either relative to ORF10-expressing cells treated only with vehicle control, or relative to control vector-expressing cells treated with d8-THCV.

FIG. 9BF provide a late apoptosis data of HEK293 (human embryonic kidney) cells transfected with i) control plasmid expressing control vector and ii) plasmid expressing viral M Protein; and then treated with 1 μM d8-THCV. d8-THCV treated cells which are transfected with control plasmid show a significant increase in late apoptosis, and d8-THCV treated cells which are transfected with plasmid expressing viral M Protein exhibited a significant increase in late apoptosis relative to viral M Protein-expressing cells treated only with vehicle control.

FIG. 10A provides Interferon Lambda 1 mRNA levels produced when cells expressing ORF8 or control vector are treated with Cannabidiol compared to vehicle control. In cells expressing ORF8, but not treated with CBD, Interferon Lambda 1 levels were not significantly elevated versus cells expressing only the empty-vector control plasmid. This highlights the problem that cells often have an inadequate innate anti-viral response to SARS-CoV-2.

In cells expressing ORF8, CBD significantly increased expression of Interferon lambda 1 at 24 hours versus treatment with vehicle alone, indicating that CBD augments this anti-viral response to ORF8.

FIG. 10B provides that CBD augmented the expression of INF-gamma in both control and ORF8-expressing cells but had a greater effect on this expression in ORF8 expressing cells.

FIG. 10C provides that in cells expressing ORF10, CBD significantly increased expression of Interferon gamma which is an indication of augmentation of the innate anti-viral response by cells. Expression of Interferon gamma is also seen in Cannabidiol treated cells transfected with a control plasmid, but to a lesser extent than in Cannabidiol-treated cells transfected with the SARS-CoV-2 gene ORF10.

FIGS. 10D and 10E provide that Cannabidiol induced both INF-lambda 1 and INF-lambda 2/3 in cells expressing M-protein, indicating that CBD augments the interferon response to this SARS-CoV-2 protein and augments this aspect of the innate intracellular anti-viral response.

FIGS. 10F to 10K provides effect of ORF8, ORF10, and M protein, with and without CBD, on gene expression of Type I INF. Data are means±SEM.

FIG. 10F provides expression of INFα in cells transfected with control plasmid (pCMV), or plasmid expressing ORF8 and treated with vehicle control (0.1% ethanol) or 2 μM CBD.

FIG. 10G provides expression of INFα in cells transfected with control plasmid (pCMV), or plasmid expressing ORF10 and treated with vehicle control (0.1% ethanol) or 2 μM CBD.

FIG. 10H provides expression of INFα in cells transfected with control plasmid (pCMV), or plasmid expressing M protein and treated with vehicle control (0.1% ethanol) or 2 μM CBD Data are means±SEM.

FIG. 10I provides expression of INFβ in cells transfected with control plasmid (pCMV), or plasmid expressing ORF8 and treated with vehicle control (0.1% ethanol) or 2 μM CBD.

FIG. 10J provides expression of INFβ in cells transfected with control plasmid (pCMV), or plasmid expressing ORF10 and treated with vehicle control (0.1% ethanol) or 2 μM CBD.

FIG. 10K provides expression of INFβ in cells transfected with control plasmid (pCMV), or plasmid expressing M protein and treated with vehicle control (0.1% ethanol) or 2 μM CBD.

FIGS. 10L to 10T provides effect of ORF8, ORF10 and M protein, with and without CBD, on gene expression of Type II and III INF.

FIG. 10L provides expression of INF γ in cells transfected with control plasmid (pCMV) or ORF8 and treated with vehicle control (0.1% ethanol) or 2 μM CBD (n=5).

FIG. 10M provides expression of INF γ in cells transfected with control plasmid (pCMV) or ORF10 and treated with vehicle control (0.1% ethanol) or 2 μM CBD (n=5).

FIG. 10N provides expression of INF γ in cells transfected with control plasmid (pCMV) or M Protein and treated with vehicle control (0.1% ethanol) or 2 μM CBD (n=5).

FIG. 10O provides expression of INF λ1 in cells transfected with control plasmid (pCMV) or ORF8 and treated with vehicle control (0.1% ethanol) or 2 μM CBD (n=5).

FIG. 10P provides expression of INF λ1 in cells transfected with control plasmid (pCMV) or ORF10 and treated with vehicle control (0.1% ethanol) or 2 μM CBD (n=5).

FIG. 10Q provides expression of INF λ1 in cells transfected with control plasmid (pCMV) or M Protein and treated with vehicle control (0.1% ethanol) or 2 μM CBD (n=5).

FIG. 10R provides expression of INF λ2/3 in cells transfected with control plasmid (pCMV) or ORF8 and treated with vehicle control (0.1% ethanol) or 2 μM CBD (n=5).

FIG. 10S provides expression of INF λ2/3 in cells transfected with control plasmid (pCMV) or ORF10 and treated with vehicle control (0.1% ethanol) or 2 μM CBD (n=5).

FIG. 10T provides expression of INF λ2/3 in cells transfected with control plasmid (pCMV) or M Protein and treated with vehicle control (0.1% ethanol) or 2 μM CBD (n=5).

FIGS. 10U to 10Z provides effect of ORF8, ORF10 and M protein, with and without 1 uM cannabinol, on gene expression of Type I INF. Data are means SEM, n=3.

FIG. 10U provides expression of INFα in cells transfected with control plasmid (pCMV), or a plasmid expressing ORF8 and treated with vehicle control (0.1% ethanol) or 1 μM cannabinol for 24 hours.

FIG. 10V provides expression of INFα in cells transfected with control plasmid (pCMV), or a plasmid expressing ORF10 and treated with vehicle control (0.1% ethanol) or 1 μM cannabinol for 24 hours.

FIG. 10W provides expression of INFα in cells transfected with control plasmid (pCMV), or a plasmid expressing M Protein and treated with vehicle control (0.10% ethanol) or 1 μM cannabinol for 24 hours.

FIG. 10X provides expression of INFβ in cells transfected with control plasmid (pCMV), or a plasmid expressing ORF8 and treated with vehicle control (0.1% ethanol) or 1 μM cannabinol for 24 hours.

FIG. 10Y provides expression of INFβ in cells transfected with control plasmid (pCMV), or a plasmid expressing ORF10 and treated with vehicle control (0.1% ethanol) or 1 μM cannabinol for 24 hours.

FIG. 10Z provides expression of INFβ in cells transfected with control plasmid (pCMV), or a plasmid expressing M Protein and treated with vehicle control (0.10% ethanol) or 1 μM cannabinol for 24 hours.

FIGS. 10AA to 10AI provides effect of ORF8, ORF10 and M protein, with and without 1 uM cannabinol, on gene expression of Type II & III IFN. Data are means±SEM, n=3.

FIG. 10AA provides expression of INFγ in cells transfected with control plasmid (pCMV) or a plasmid expressing ORF8 and treated with vehicle control (0.1% ethanol) or 1 μM cannabinol for 24 hours.

FIG. 10AB provides expression of INFγ in cells transfected with control plasmid (pCMV) or a plasmid expressing ORF10 and treated with vehicle control (0.1% ethanol) or 1 μM cannabinol for 24 hours.

FIG. 10AC provides expression of INFγ in cells transfected with control plasmid (pCMV), or a plasmid expressing M protein and treated with vehicle control (0.10% ethanol) or 1 μM cannabinol for 24 hours.

FIG. 10AD provides expression of INF λ1 in cells transfected with control plasmid (pCMV), or a plasmid expressing ORF8 and treated with vehicle control (0.1% ethanol) or 1 μM cannabinol for 24 hours.

FIG. 10AE provides expression of INF λ1 in cells transfected with control plasmid (pCMV) or a plasmid expressing ORF10 and treated with vehicle control (0.1% ethanol) or 1 μM cannabinol for 24 hours.

FIG. 10AF provides expression of INF λ1 in cells transfected with control plasmid (pCMV) or a plasmid expressing M protein and treated with vehicle control (0.1% ethanol) or 1 μM cannabinol for 24 hours.

FIG. 10AG provides expression of INF λ 2-3 in cells transfected with control plasmid (pCMV) or a plasmid expressing ORF8 and treated with vehicle control (0.1% ethanol) or 1 μM cannabinol for 24 hours.

FIG. 10AH provides expression of INF λ 2-3 in cells transfected with control plasmid (pCMV) or plasmids expressing ORF10 and treated with vehicle control (0.1% ethanol) or 1 μM cannabinol for 24 hours.

FIG. 10AI provides expression of INF λ 2-3 in cells transfected with control plasmid (pCMV) or a plasmid expressing M protein and treated with vehicle control (0.1% ethanol) or 1 μM cannabinol for 24 hours.

FIGS. 10AJ to 10AO provides effect of ORF8, ORF10, or M protein, with and without Cannabigerol (CBG), on gene expression of Type I INF. Data are means SEM, n=3

FIG. 10AJ provides expression of INFα in cells transfected with control plasmid (pCMV) or a plasmid expressing ORF8 and treated with vehicle control (0.1% ethanol) or 1 μM CBG for 24 hours.

FIG. 10AK provides expression of INFα in cells transfected with control plasmid (pCMV) or a plasmid expressing ORF10 and treated with vehicle control (0.1% ethanol) or 1 μM CBG for 24 hours.

FIG. 10AL provides expression of INFα in cells transfected with control plasmid (pCMV) or a plasmid expressing M protein and treated with vehicle control (0.1% ethanol) or 1 μM CBG for 24 hours.

FIG. 10AM provides expression of INFβ in cells transfected with control plasmid (pCMV) or a plasmid expressing ORF8 and treated with vehicle control (0.1% ethanol) or 1 μM CBG for 24 hours.

FIG. 10AN provides expression of INFβ in cells transfected with control plasmid (pCMV) or a plasmid expressing ORF10 and treated with vehicle control (0.1% ethanol) or 1 μM CBG for 24 hours.

FIG. 10AO provides expression of INFβ in cells transfected with control plasmid (pCMV) or a plasmid expressing M protein and treated with vehicle control (0.1% ethanol) or 1 μM CBG for 24 hours.

FIGS. 10AP to 10AX provides effect of ORF8, ORF10 and M protein, with and without cannabigerol (CBG), on gene expression of Type II & III IFN. Data are means±SEM, n=3.

FIG. 10AP provides expression of INF γ in cells transfected with control plasmid (pCMV) or a plasmid expressing ORF8 and treated with vehicle control (0.1% ethanol) or 1 μM CBG for 24 hours.

FIG. 10AQ provides expression of INF γ in cells transfected with control plasmid (pCMV) or a plasmid expressing ORF10 and treated with vehicle control (0.1% ethanol) or 1 μM CBG for 24 hours.

FIG. 10AR provides expression of INF γ in cells transfected with control plasmid (pCMV) or a plasmids expressing M protein and treated with vehicle control (0.1% ethanol) or 1 μM CBG for 24 hours.

FIG. 10AS provides expression of INF λ1 in cells transfected with control plasmid (pCMV) or a plasmid expressing ORF8 and treated with vehicle control (0.1% ethanol) or 1 μM CBG for 24 hours.

FIG. 10AT provides expression of INF λ1 in cells transfected with control plasmid (pCMV) or a plasmid expressing ORF10 and treated with vehicle control (0.1% ethanol) or 1 μM CBG for 24 hours.

FIG. 10AU provides expression of INF λ1 in cells transfected with control plasmid (pCMV) or a plasmid expressing M protein and treated with vehicle control (0.1% ethanol) or 1 μM CBG for 24 hours.

FIG. 10AV provides expression of INF λ2-3 in cells transfected with control plasmid (pCMV) or plasmid expressing ORF8 and treated with vehicle control (0.1% ethanol) or 1 μM CBG for 24 hours.

FIG. 10AW provides expression of INF λ2-3 in cells transfected with control plasmid (pCMV) or plasmid expressing ORF10 and treated with vehicle control (0.1% ethanol) or 1 μM CBG for 24 hours.

FIG. 10AX provides expression of INF λ2-3 in cells transfected with control plasmid (pCMV) or a plasmid expressing M protein and treated with vehicle control (0.1% ethanol) or 1 μM CBG for 24 hours.

FIG. 11A provides that Mx1 (Dynamin-Like GTPase myxovirus resistance protein 1) another interferon stimulated gene, is more highly expressed when cells transfected with ORF8 protein are treated with Cannabidiol for 24 hours, highlighting that Cannabidiol in combination with this SARS-CoV-2 protein augments this anti-viral response.

FIG. 11B provides that cells transfected with both control plasmid and M protein and treated with cannabidiol have exhibited greater expression of Mx1. Cannabidiol induces Mx1 gene expression in cells transfected with M-protein and treated with Cannabidiol to a significantly greater extent than in cells treated with Cannabidiol but expressing only control plasmid.

FIGS. 11C to 11E provides effect of ORF8, ORF10, or M protein, with and without CBD, on gene expression of Mx after 14 hours. Data are means±SEM.

FIG. 11C provides expression of Mx in cells transfected with control plasmid (pCMV), ORF8 and treated with vehicle control (0.1% ethanol) or 2 μM CBD for 14 h (n=5).

FIG. 11D provides expression of Mx in cells transfected with control plasmid (pCMV), ORF10 and treated with vehicle control (0.1% ethanol) or 2 μM CBD for 14 h (n=5).

FIG. 11E provides expression of Mx in cells transfected with control plasmid (pCMV), M protein, and treated with vehicle control (0.1% ethanol) or 2 μM CBD for 14 h (n=5).

FIG. 11F provides expression of Mx in cells transfected with control plasmid (pCMV), ORF8 and treated with vehicle control (0.1% ethanol) or 1 μM CBD for 24 h (n=5).

FIG. 11G provides expression of Mx in cells transfected with control plasmid (pCMV), ORF10 and treated with vehicle control (0.1% ethanol) or 1 μM CBD for 24 h (n=5).

FIG. 11H provides expression of Mx in cells transfected with control plasmid (pCMV), M protein and treated with vehicle control (0.1% ethanol) or 1 μM CBD for 48 h (n=5).

FIGS. 11I and 11J are same as 11C and 11D (they are repeated) FIG. 12A provides that cannabidiol significantly increases expression of IFIT1 either in cells transfected with M protein or control plasmid, and therefore may help to prime the innate cellular immune system to enhance ability to launch an anti-viral defense.

FIGS. 12B to 12D provides effect of ORF8, ORF10, or M protein, with and without CBD, on gene expression of IFIT. Data are means±SEM.

FIG. 12B provides expression of IFIT in cells transfected with control plasmid (pCMV), ORF8 and treated with vehicle control (0.1% ethanol) or 2 μM CBD for 14 h (n=5).

FIG. 12C provides expression of IFIT in cells transfected with control plasmid (pCMV), ORF10 and treated with vehicle control (0.1% ethanol) or 2 μM CBD for 14 h (n=5).

FIG. 12D provides expression of IFIT in cells transfected with control plasmid (pCMV), M protein, and treated with vehicle control (0.1% ethanol) or 2 μM CBD for 14 h (n=5).

FIG. 12E provides expression of IFIT in cells transfected with control plasmid (pCMV), ORF8 and treated with vehicle control (0.1% ethanol) or 1 μM CBD for 48 h (n=5).

FIG. 12F provides expression of IFIT in cells transfected with control plasmid (pCMV), ORF10 and treated with vehicle control (0.1% ethanol) or 1 μM CBD for 48 h (n=5).

FIG. 12G provides expression of IFIT in cells transfected with control plasmid (pCMV), M protein and treated with vehicle control (0.1% ethanol) or 1 μM CBD for 48 h (n=5).

FIG. 13A provides a highly significant increase in the expression of OAS1 (Oligoadenylate synthetases 1) gene in cells transfected with ORF8 protein and treated with Cannabidiol relative to all other groups and treatments.

FIG. 13B provides expression of OAS1 in cells transfected with a control plasmid or plasmid expressing ORF10 and treated with Cannabidiol. Treatment with Cannabidiol significantly increased the induction of OAS1 in cells transfected with ORF10 or control plasmid relative to treatment with vehicle alone (i.e. without Cannabidiol).

FIG. 13C provides that the cells transfected with either control plasmid or M protein and treated with Cannabidiol have exhibited significantly greater expression of OAS1 gene compared to their respective vehicle-treated cells.

FIGS. 13D to 13F provides effect of ORF8, ORF10 and M protein, with and without CBD, on gene expression of OAS1. Data are means±SEM. *P<0.05, ***P<0.001, ****P<0.0001.

FIG. 13D provides expression of OAS1 in cells transfected with control plasmid (pCMV), ORF8 and treated with vehicle control (0.1% ethanol) or 2 μM CBD for 14 h (n=5).

FIG. 13E provides expression of OAS1 in cells transfected with control plasmid (pCMV), ORF10 and treated with vehicle control (0.1% ethanol) or 2 μM CBD for 14 h (n=5).

FIG. 13F provides expression of OAS1 in cells transfected with control plasmid (pCMV), M protein, and treated with vehicle control (0.1% ethanol) or 2 μM CBD for 14 h (n=5).

FIGS. 13G to 13I provides effect of ORF8, ORF10, or M protein, with and without 1 uM cannabinol, on gene expression of OAS1. Data are means±SEM. *P<0.05, ***P<0.001, ****P<0.0001

FIG. 13G provides expression of OAS1 in cells transfected with control plasmid (pCMV), ORF8 and treated with vehicle control (0.1% ethanol) or 1 μM cannabinol for 24 h (n=3).

FIG. 13H provides expression of OAS1 in cells transfected with control plasmid (pCMV), ORF10 and treated with vehicle control (0.1% ethanol) or 1 μM cannabinol for 24 h (n=3).

FIG. 13I provides expression of OAS1 in cells transfected with control plasmid (pCMV), M protein, and treated with vehicle control (0.1% ethanol) or 1 μM cannabinol for 24 h (n=3).

FIGS. 13J to 13L provides effect of ORF8, ORF10, or M protein, with and without 1 uM cannabigerol (CBG), on gene expression of OAS1. Data are means±SEM. *P<0.05, ***P<0.001, ****P<0.0001.

FIG. 13J provides expression of OAS1 in cells transfected with control plasmid (pCMV), ORF8 and treated with vehicle control (0.1% ethanol) or 1 μM CBG for 24 h (n=3).

FIG. 13K provides expression of OAS1 in cells transfected with control plasmid (pCMV), ORF10 and treated with vehicle control (0.1% ethanol) or 1 μM CBG for 24 h (n=3).

FIG. 13L provides expression of OAS1 in cells transfected with control plasmid (pCMV), M protein and treated with vehicle control (0.1% ethanol) or 1 μM CBG for 24 h (n=3).

FIGS. 14A to 14C provides effect of ORF8, ORF10 and M protein, with and without CBD, on gene expression of OAS2. Data are means±SEM. *P<0.05, ***P<0.001, ****P<0.0001.

FIG. 14A provides expression of OAS2 in cells transfected with control plasmid (pCMV), ORF8 and treated with vehicle control (0.1% ethanol) or 2 μM CBD for 14 h (n=5).

FIG. 14B provides expression of OAS2 in cells transfected with control plasmid (pCMV), ORF10 and treated with vehicle control (0.10% ethanol) or 2 μM CBD for 14 h (n=5).

FIG. 14C provides expression of OAS2 in cells transfected with control plasmid (pCMV) or M protein, and treated with vehicle control (0.1% ethanol) or 2 μM CBD for 14 h (n=5).

FIGS. 14D to 14F provides effect of ORF8, ORF10, or M protein, with and without 1 uM cannabinol, on gene expression of OAS2. Data are means±SEM. *P<0.05, ***P<0.001, ****P<0.0001

FIG. 14D provides expression of OAS2 in cells transfected with control plasmid (pCMV), ORF8 and treated with vehicle control (0.1% ethanol) or 1 μM cannabinol for 24 h (n=3).

FIG. 14E provides expression of OAS2 in cells transfected with control plasmid (pCMV), ORF10 and treated with vehicle control (0.1% ethanol) or 1 μM cannabinol for 24 h (n=3).

FIG. 14F provides expression of OAS2 in cells transfected with control plasmid (pCMV), M protein, and treated with vehicle control (0.1% ethanol) or 1 μM cannabinol for 24 h (n=3).

FIGS. 14G to 14I provides effect of ORF8, ORF10, or M protein, with and without 1 uM cannabigerol (CBG), on gene expression of OAS2. Data are means±SEM. *P<0.05, ***P<0.001, ****P<0.0001.

FIG. 14G provides expression of OAS2 in cells transfected with control plasmid (pCMV), ORF8 and treated with vehicle control (0.1% ethanol) or 1 μM CBG for 24 h (n=3).

FIG. 14H provides expression of OAS2 in cells transfected with control plasmid (pCMV), ORF10 and treated with vehicle control (0.1% ethanol) or 1 μM CBG for 24 h (n=3).

FIG. 14I provides expression of OAS2 in cells transfected with control plasmid (pCMV), M protein and treated with vehicle control (0.1% ethanol) or 1 μM CBG for 24 h (n=3).

FIGS. 15A to 15C provides effect of ORF8, ORF10 and M protein, with and without CBD, on gene expression of OAS3. Data are means±SEM. *P<0.05, ***P<0.001, ****P<0.0001.

FIG. 15A provides expression of OAS3 in cells transfected with control plasmid (pCMV), ORF8 and treated with vehicle control (0.1% ethanol) or 2 μM CBD for 14 h (n=5).

FIG. 15B provides expression of OAS3 in cells transfected with control plasmid (pCMV), ORF10 and treated with vehicle control (0.1% ethanol) or 2 μM CBD for 14 h (n=5).

FIG. 15C provides expression of OAS3 in cells transfected with control plasmid (pCMV) or M protein, and treated with vehicle control (0.1% ethanol) or 2 μM CBD for 14 h (n=5).

FIGS. 15D to 15F provides effect of ORF8, ORF10, or M protein, with and without 1 uM cannabinol, on gene expression of OAS3. Data are means±SEM. *P<0.05, ***P<0.001, ****P<0.0001

FIG. 15D provides expression of OAS3 in cells transfected with control plasmid (pCMV), ORF8 and treated with vehicle control (0.1% ethanol) or 1 μM cannabinol for 24 h (n=3).

FIG. 15E provides expression of OAS3 in cells transfected with control plasmid (pCMV), ORF10 and treated with vehicle control (0.1% ethanol) or 1 μM cannabinol for 24 h (n=3).

FIG. 15F provides expression of OAS3 in cells transfected with control plasmid (pCMV), M protein, and treated with vehicle control (0.1% ethanol) or 1 μM cannabinol for 24 h (n=3).

FIGS. 15G to 15I provides effect of ORF8, ORF10, or M protein, with and without 1 uM cannabigerol (CBG), on gene expression of OAS3. Data are means±SEM. *P<0.05, ***P<0.001, ****P<0.0001.

FIG. 15G provides expression of OAS3 in cells transfected with control plasmid (pCMV), ORF8 and treated with vehicle control (0.1% ethanol) or 1 μM CBG for 24 h (n=3).

FIG. 15H provides expression of OAS3 in cells transfected with control plasmid (pCMV), ORF10 and treated with vehicle control (0.1% ethanol) or 1 μM CBG for 24 h (n=3).

FIG. 15I provides expression of OAS3 in cells transfected with control plasmid (pCMV), M protein and treated with vehicle control (0.1% ethanol) or 1 μM CBG for 24 h (n=3).

FIGS. 16A to 16C provides effect of ORF8, ORF10 and M protein, with and without CBD, on gene expression of OASL. Data are means±SEM. *P<0.05, ***P<0.001, ****P<0.0001.

FIG. 16A provides expression of OASL in cells transfected with control plasmid (pCMV), ORF8 and treated with vehicle control (0.1% ethanol) or 2 μM CBD for 14 h (n=5).

FIG. 16B provides expression of OASL in cells transfected with control plasmid (pCMV), ORF10 and treated with vehicle control (0.1% ethanol) or 2 μM CBD for 14 h (n=5).

FIG. 16C provides expression of OASL in cells transfected with control plasmid (pCMV) M protein, and treated with vehicle control (0.1% ethanol) or 2 μM CBD for 14 h (n=5).

FIGS. 16D to 16F provides effect of ORF8, ORF10, or M protein, with and without 1 uM cannabinol, on gene expression of OASL. Data are means±SEM. *P<0.05, ***P<0.001, ****P<0.0001

FIG. 16D provides expression of OASL in cells transfected with control plasmid (pCMV), ORF8 and treated with vehicle control (0.1% ethanol) or 1 μM cannabinol for 24 h (n=3).

FIG. 16E provides expression of OASL in cells transfected with control plasmid (pCMV), ORF10 and treated with vehicle control (0.1% ethanol) or 1 μM cannabinol for 24 h (n=3).

FIG. 16F provides expression of OASL in cells transfected with control plasmid (pCMV), M protein, and treated with vehicle control (0.1% ethanol) or 1 μM cannabinol for 24 h (n=3).

FIGS. 16G to 16I provides effect of ORF8, ORF10, or M protein, with and without 1 uM cannabigerol (CBG), on gene expression of OASL. Data are means±SEM. *P<0.05, ***P<0.001, ****P<0.0001.

FIG. 16G provides expression of OASL in cells transfected with control plasmid (pCMV), ORF8 and treated with vehicle control (0.1% ethanol) or 1 μM CBG for 24 h (n=3).

FIG. 16H provides expression of OASL in cells transfected with control plasmid (pCMV), ORF10 and treated with vehicle control (0.1% ethanol) or 1 μM CBG for 24 h (n=3).

FIG. 16I provides expression of OASL in cells transfected with control plasmid (pCMV), M protein and treated with vehicle control (0.1% ethanol) or 1 μM CBG for 24 h (n=3).

### BACKGROUND OF THE ART

Viral proteins usually play critical roles in interfering with the host acquired immune response, but can also directly interfere with anti-viral innate immune responses mediated directly within infected cells that are meant to stop viral replication and spread. The pandemic of coronavirus disease 2019 (COVID-19) caused by the 2019 novel coronavirus (2019-nCoV or SARS-CoV-2) infection has become a Public Health Emergency of International Concern (PHEIC). SARS-CoV-2 is highly pathogenic in human, having posed immeasurable public health challenges to the world.

SARS CoV-2 is related to an earlier strain that also causes respiratory disease in humans, SARS CoV. Prior characterization of SARS CoV has facilitated decoding the SARS CoV2 genome.

Genomic products of the SARS CoV-2 genome are designated in lower case letters, in italics (e.g. orf10), while viral genes are designated in upper case letters (e.g. ORF10).

The pandemic of coronavirus disease 2019 (COVID-19) caused by the 2019 novel coronavirus (2019-nCoV or SARS-CoV-2) infection has created havoc by infecting more than 127 million individuals across the world and by causing around 3 million deaths as of Mar. 29, 2021, with both the numbers of cases and deaths still climbing. It has been reported that some coronavirus proteins play an important role in modulating innate immunity of the host, but few studies have been conducted on SARS-CoV-2.

Several independent research studies by Lu, R. et al.; Zhou, P. et al, Xu, J. et al., provided that SARS-CoV-2 shares almost 80% of the genome with SARS-CoV. Lu, R. et al further provided that almost all encoded proteins of SARS-CoV-2 are homologous to SARS-CoV proteins.

(SARS)-CoV was identified as the etiologic agent of the 2002-3 international SARS outbreak. Chong-Shan Shi et al in a paper published in Journal of Immunology (2014) provides an insightful study on how SARS evades innate immune responses to cause human disease.

According to Shi, a protein encoded by SARS-CoV designated as open reading frame-9b (ORF-9b) plays multiple roles as follows:
1. It localizes to mitochondria and causes mitochondrial elongation by triggering ubiquitination and proteasomal degradation of dynamin-like protein (DRP1), a host protein involved in mitochondrial fission;
2. It acts on mitochondria and targets the mitochondrial-associated adaptor molecule viz. Mitochondrial antiviral signaling protein (signalosome) (MAVS) by usurping poly(C)-binding protein 2 (PCBP2) and the HECT domain E3 ligase AIP4 to trigger the degradation of MAVS, TRAF3, and TRAF6. This severely limits host cell interferon responses.
3. Transient ORF-9b expression led to a strong induction of autophagy in cells.

Shi reports as follows:
“These results indicate that SARS-CoV ORF-9b manipulates host cell mitochondria and mitochondrial function to help evade host innate immunity. This study has uncovered an important clue to the pathogenesis of SARS-CoV infection and illustrates the havoc that a small open reading frame can cause in cells.”

All viral proteins of SARS-COV-2 viz. NSP1, NSP2, NSP3, NSP4, NSP5, NSP6, NSP7, NSP8, NSP9, NSP10, NSP11, NSP12, NSP13, NSP14, NSP15, NSP16, S protein, ORF3a, E protein, M protein, ORF6, ORF7a, ORF7b, ORF8, N protein, ORF10 are being extensively researched for development of novel therapeutics to treat Covid-19 (Gordon, D. E et al, 2020).

Jin-Yan Li et al (2020) in their recently published “Short Communication” in Virus Research 286 (2020) 198074, screened the viral proteins of SARS-CoV-2.

Li et al (2020) reports the following mechanism which is triggered upon viral infection.
i) Binding of several transcription factors, such as IRF-3 and NF-κB, to the interferon promoter to stimulate type I IFN (IFN-α/β) expression (Garcia-Sastre and Biron, 2006) upon virus infection;
ii) Secretion of Interferon and its binding with its receptor;
iii) initiation of the JAK/STAT pathway and inducing the nucleus translocation of IFN-responsive transcriptional factors upon binding of interferon with its receptors;
iv) activation of genes containing interferon-stimulated response elements (ISREs) in their promoters, resulting in the expression of a set of IFN-stimulated genes (ISGs) establishing an antiviral state (Catanzaro et al., 2020).

Li further states that in response to this powerful selective environment, many viruses from diverse families, including filoviruses, poxviruses, influenza viruses, flaviviruses, and coronaviruses (CoVs), have evolved multiple passive and active mechanisms to avoid induction of the antiviral type I interferon, and they could optimize the intracellular resource for efficient virus replication (Volk et al., 2020).

Li et al found that the viral ORF6, ORF8 and nucleocapsid proteins were potential inhibitors of the type I interferon signaling pathway, a key component for antiviral response of host innate immunity. All the three proteins showed strong inhibition on type I interferon (IFN-0) and NF-κB-responsive promoters. Further examination by Li revealed that these proteins were able to inhibit the interferon-stimulated response element (ISRE) after infection with Sendai virus, while only ORF6 and ORF8 proteins were able to inhibit the ISRE after treatment with interferon beta.

SARS-CoV-2 ORF6, ORF8, N and ORF3b are potent interferon antagonists, and in the early stages of SARS-CoV-2 infection, delayed release of IFNs would hinder the host's antiviral response and then benefit virus replication. This, followed by the rapidly increased cytokines and chemokines attract inflammatory cells, such as neutrophils and monocytes, resulting in excessive immune infiltration causing tissue damage.

Khailany et al refers to an article by Koyama et al, 2020, wherein Koyama finds that ORF10, a short 38-residue peptide from SARS-CoV-2 genome is not homologous with other proteins in the NCBI repository and Khailany further states that since ORF10 doesn't have any comparative proteins in the NCBI repository, it is one of a kind protein, which can be used to distinguish the infection more rapidly than PCR based strategies, but the further characterization of this protein is strongly required.

Interestingly, a paper available on chemrxiv.org by Seema Mishra titled “ORF10: Molecular insights into the contagious nature of pandemic novel coronavirus 2019-nCoV” emphasized on the fact that ORF10 is an unknown protein with no homology to any known protein in organisms present till date. She further conducted immunoinformatics studies through which, it has been observed that among all ten 2019-nCoV proteins, ORF10 presents amongst the highest number of immunogenic, promiscuous CTL epitopes. (Cytotoxic T Lymphocytes).

While linking ORF10 to a contagious nature of pandemic novel coronavirus 2019-nCoV, she states as follows:
Through immunoinformatics studies, it has been observed that among all ten 2019-nCoV proteins, ORF10 presents amongst the highest number of immunogenic, promiscuous CTL epitopes. These epitopes are part of a cluster with HTL epitopes, suggesting that there is a high degree of epitope conservation in ORF10. Conservation of protein sequence across organisms is not seen, and there is no known structural template on which to model and derive a structure to get structural and functional insights. Because there is altogether no conservation of its sequence, or structure, it may be presented as a novel protein to the immune system. Further, the human body may not have been able to utilize any memory B and T cells generated against other microorganisms to target ORF10 and fight this pathogen, contributing to its deadly, contagious nature.”.

Additionally, ORF8 protein is another protein that is not homologous with other proteins in the SARS CoV genome (Xu, J. et al., Viruses 2020, 12, 244), although it does show very low homology to proteins encoded by other related viruses (Tang, X. et al. National Science Review 2020, 7, 1012-1023). The SARS CoV-2 ORF8 protein is of a particular interest due to the recent finding that it is potential inhibitor of type I interferon signaling pathway, a key component for antiviral response of host innate immunity.

The gene for orf8 (Accession YP_009724396.1, UniProt ID P0DTC8⋅NS8_SARS2), is encoded at the 3′ end of the SARS CoV-2 genome. It results in a protein that is 121 amino acids long, with the N-terminal region forming a predicted signal peptide identifying a cleavage site at aa 15 (Target P-2.0 prediction). The predicted subcellular localization (using PSORTII, https://psort.hgc.jp/form2.html) is extracellular (55.6%).

However, over 80 cellular proteins that potentially interact with ORF8 (www.ebi.ac/uk/interact/interactors/id:P0DTC8) have been identified. These include mitochondrial proteins involved in metabolism, and cardiolipin and lipid synthesis (e.g. mitochondrial glutamate carrier 1, mitochondrial ATP synthase subunits alpha and beta, alpha trifunctional protein, and various dehydratases and enolases), Golgi proteins (e.g. Coatomer subunits (/®/,©, etc), endoplasmic reticulum (ER) proteins (e.g. ER lectin 1, ER membrane protein complex subunit 1, etc), proteasomal proteins (e.g. 26S proteasome non-ATPase regulatory subunit 6, proteasome subunit alpha type-7), nuclear proteins (e.g. EIF3A, RBP2, etc.), and others.

ORF10 protein, being an unknown protein with no homology to any known protein in organisms present till date, and due to its unique association with SARS-CoV-2, also serves an interesting candidate.

ORF10 (Accession YP_009725255.1, UniProt ID A0A663DJA2*), is predicted by PSORT II to likely be cytoplasmic (56.5% probability), but also mitochondrial (21.7%), nuclear (13%), secretory system vesicle-associated (4.3%) or ER-associated (4.3%). This viral protein is small, at only 38 amino acids, and has a predicted N-terminal transmembrane helix spanning amino acids 5-19.

Protein interaction data from the IntAct database (https://www.ebi.ac.uk/intact/interactors/id:AOA663DJA2*) indicates only 30 potential interactors. It is notable, however, that there are several common interactors between ORF8 protein and ORF10 protein, including mitochondrial, Golgi, and endoplasmic reticulum proteins.

The membrane glycoprotein (M protein, Accession YP_009724393.1)) is a structural protein that is highly conserved across all beta-coronaviruses, but has been found to have some sequence variants in the SARS CoV-2 virus, with at least 7 amino acid substitutions identified thus far (M. Bianchi et al, BioMed Research International Vol 2020 Article ID 4389089). The M protein may be important for viral entry, replication, and particle assembly within host cells, as well as for viral budding. Data from an interaction study also suggests that M protein may interfere with mitochondrial metabolism (https://doi.org/10.1038/s41586-020-2286-9) and additional cellular processes.

There are a number of encoded non-structural proteins in the SARS CoV-2 genome. The non-structural protein 5 (NSP5) is encoded in open reading frame 1a (orf1a) that produces a polypeptide (Accession #YP_009725295.1) and orf1ab (polypeptide Accession #YP_009724389.1), which are further processed to yield non-structural proteins including NSP5. A recent interaction study has suggested based on protein-protein interactions that NSP5, which is the main protease of the SARS CoV-2 genome, may affect the ability of proteins to target the mitochondria and cause oxidative stress, and may be targeted therapeutically by anti-oxidant drugs, although this has not yet been shown experimentally.

A lack of basic knowledge about SARS CoV-2 is a limiting factor for the development of novel therapeutics to treat this disease. Although SARS-CoV-2 has been observed to share almost 80% of the genome with SARS-CoV (Catanzaro 2020), given that there are differences in the infectivity, host interaction, and pathogenicity between these two viruses (2), ORF8 protein and ORF10 protein are of significant interest, as well as M protein and NSP5, among the other known individual proteins in the SARS CoV-2 genome.

In recent times, interest in cannabidiol (CBD), which is a nonpsychoactive constituent of marijuana with potent antioxidant and anti-inflammatory effects, is rising exponentially.

CBD had been found to modulate translocation of various cellular proteins including transcription factors. CBD exposure rapidly increased TRPV2 protein expression and promoted its translocation to the cell surface of BV-2 cells (Samia Hassan 2014).

Chong-Shan Shi et al provides how a protein encoded by SARS-CoV designated as open reading frame-9b (ORF-9b) localizes to mitochondria and causes mitochondrial elongation by triggering ubiquitination and proteasomal degradation of dynamin-like protein (DRP1), a host protein involved in mitochondrial fission (Shi et al 2014). CBD has been found to rescue levels of dynamin 1 that are reduced in iron-overloaded cells (da Silva V K et al 2014).

Enkui Hao et al reports protective effects of CBD against doxycycline-induced cardiotoxicity and cardiac dysfunction by
(i) attenuating oxidative and nitrative stress, (ii) improving mitochondrial function,
(iii) enhancing mitochondrial biogenesis, (iv) decreasing cell death and expression of MMPs and (v) decreasing myocardial inflammation.

CBD had been found to modulate translocation of various cellular proteins including transcription factors (Huang Y et al, 2019) and membrane cation channels (Hassan S et al, 2014).

CBD has been found to function in the modulation of mitochondrial calcium metabolism, mitochondrially-mediated apoptosis, mitochondrial ferritin regulation, the electron transport chain, and mitochondrial biogenesis and fission (da Silva V K, 2018; Hao E et al, 2015; McKallip R J et al, 2006; Ryan D et al, 2009 and Valvassori S S et al, 2013).

In the inhouse work on adenovirus (unpublished data), inventors have found lower complex I activity in infected cells. However, it has been shown that CBD treatment of rats increased complex I, II, III, and IV activity, likely due to enhanced accumulation of calcium inside the mitochondria, which increased the activity of calcium-sensitive dehydrogenases and promoted availability of NADH for oxidative phosphorylation (Valvassori S S et al, 2013).

Various researchers have shown that CBD shows significant promise for the treatment of numerous cancers, based primarily on evidence of induction of a pro-apoptotic effect (Jeong S et al, 2019; Jeong S, Yun H K et al, 2019; Sultan A S et al, 2018). In inhouse work, inventors have found that in metabolically dysregulated cells, CBD decreases cell death, and has no effect in normal cells (unpublished data). The same is also observed by Olih A et al, 2016; and Solinas M et al, 2012.

Cell type may also be a factor in determining response. In an in vivo model of hypoxic-ischemic injury, mouse forebrain tissues subjected to oxygen-glucose deprivation had a 5-fold increase in caspase 9 activation that was attenuated nearly 50% by 100 μM CBD (Castillo A et al, 2010), while 5 μM CBD also significantly attenuated apoptosis and oxidative stress in cultured HT22 hippocampal neurons subjected to oxygen-glucose deprivation (Sun S et al, 2017).

Whether CBD, or other cannabinoids, can attenuate potential pro-apoptotic effects of viral proteins requires direct investigation.

Following data is found reported on the modulation of lipid metabolism by CBD:
a. CBD is reported to stimulate sphingomyelin hydrolysis in cells cultured from a patient with Niemann-Pick disease, suggesting that it may help to relieve symptoms caused by accumulation (Burstein S et al, 1984).
b. In a paper published almost 40 years ago, it was also found that cannabidiol and other cannabinoids dose-dependently inhibited cholesterol esterification in cultured human fibroblasts, without affecting triacylglycerol or phospholipid synthesis (Comicelli J A, et al 1981).
c. CBD treatment of cultured mouse microglial cells also alters the accumulation of specific species of N-acylethanolamines (N-AE) in membrane lipid rafts (Rimmerman N et al, 2012). Although minor components, N-AE are highly bioactive. As docking sites for membrane-bound proteins, and ‘scaffolding sites’ for the assembly of signaling complexes, lipid rafts are important sites in cells for physiological and pathophysiological regulation.
d. Regulation of lipid rafts may also have specific importance in COVID-19. The ACE2 receptor, which binds the Spike protein of SARS CoV-2 to initiate cellular entry and infection, is located within cholesterol-rich lipid domains (Lu Y et al, 2008).

Since both sphingomyelin and free (i.e. unesterified) cholesterol are significant components of lipid rafts, these reports indicate a potential role for CBD in regulation of these membrane subdomains.

Cannabidiol is the main cannabinoid constituent of Cannabis sativa plant. It binds very weakly to CB1 and CB2 receptors. CANNABIDIOL does not induce psychoactive or cognitive effects and is well tolerated without side effects in humans, thus making it a putative therapeutic target. In the United States, the Cannabidiol drug Epidiolex was approved by the Food and Drug Administration in 2018 for the treatment of two epilepsy disorders: Dravet Syndrome and Lennox/Gasteaut Syndrome.

Cannabidiol is designated chemically as 2-[(1R,6R)-3-Methyl-6-(1-methylethenyl)-2-cyclohexen-1-yl]-5-pentyl-1,3-benzenediol. The chemical structure is as follows.

The U.S. Pat. No. 6,410,588 discloses the use of Cannabidiol to treat inflammatory diseases.

The PCT publication no. WO2001095899A2 relates to Cannabidiol derivatives and to pharmaceutical compositions comprising Cannabidiol derivatives being anti-inflammatory agents having analgesic, antianxiety, anticonvulsive, neuroprotective, antipsychotic and anticancer activity.

Cannabidiol is approved as an anti-seizure drug (Barnes, 2006; Devinsky et al., 2017). Cannabidiol lacks adverse cardiac toxicity and ameliorates diabetes/high glucose induced deleterious cardiomyopathy (Cunha et al., 1980; Izzo, Borrelli, Capasso, Di Marzo & Mechoulam, 2009; Rajesh et al., 2010).

Cannabidiol has been shown to be effective in protecting endothelial function and integrity in human coronary artery endothelial cells (HCAECs) by Rajesh M et al.

They have proposed following action of Cannabidiol by inhibiting
Reactive oxygen species production by mitochondria;
NF-κB activation;
Transendothelial migration of monocytes;
Monocyte-endothelial adhesion in HCAECs.

Nagarkatti et al provides as follows:
Cannabinoids, the active components of Cannabis sativa, and endogenous cannabinoids mediate their effects through activation of specific cannabinoid receptors known as cannabinoid receptor 1 and 2 (CBT and CB2).
The cannabinoid system has been shown both in vivo and in vitro to be involved in regulating the immune system through its immunomodulatory properties.
Cannabinoids suppress inflammatory response and subsequently attenuate disease symptoms. This property of cannabinoids is mediated through multiple pathways such as induction of apoptosis in activated immune cells, suppression of cytokines and chemokines at inflammatory sites and upregulation of FoxP3 regulatory T cells.
Cannabinoids have been tested in several experimental models of autoimmune disorders such as multiple sclerosis, rheumatoid arthritis, colitis and hepatitis and have been shown to protect the host from the pathogenesis through induction of multiple anti-inflammatory pathways.

Vuolo et al has demonstrated Role of Cannabidiol Treatment in Animal Model of Asthma. The levels of all 6 cytokines implicated in asthma viz. TNFα, IL-6, IL-4, IL-13, IL-10, and IL-5 were determined in Control animals, asthma induced animals and asthma induced animals treated with Cannabidiol. Induced asthma has increased all 6 cytokines; however, in animal group treated with CBD, levels of all cytokines has been reduced significantly. This action of Cannabidiol is very important not only in asthma but in other conditions where a rise in cytokines is reported. Recent studies by Huang, C. et al. have shown that in addition to dyspnea, hypoxemia, and acute respiratory distress, lymphopenia, and cytokine release syndrome are also important clinical features in patients with severe SARS-CoV-2 infection. Thus, Cannabidiol is also proposed as a treatment for Covid-19 due to its ability to reduce cytokines.

Interestingly further, very recently Zhou Sirui et al (Zhou Sirui et al, 2021) states as follows:
“ . . . we found that an s.d. increase in OAS1 levels was associated with reduced COVID-19 death or ventilation (odds ratio (OR) 0.54, P=7×10-8), hospitalization (OR=0.61, P=8×10-8) and susceptibility (OR=0.78, P=8×10-6). Measuring OAS1 levels in 504 individuals, we found that higher plasma OAS1 levels in a non-infectious state were associated with reduced COVID-19 susceptibility and severity. Further analyses suggested that a Neanderthal isoform of OAS1 in individuals of European ancestry affords this protection. Thus, evidence from MR and a case-control study support a protective role for OAS1 in COVID-19 adverse outcomes. Available pharmacological agents that increase OAS1 levels could be prioritized for drug development.”

### DETAILED DESCRIPTION OF THE INVENTION

### Definition of Terms

The terms Cannabidiol and CBD are used synonymously.

Cannabinoid is one or more of a natural, semisynthetic, biosynthetic, synthetic or combinations thereof. The term Cannabinoid means a Cannabinoid including but not restricted to Cannabidiol (CBD), Cannabigerol (CBG), Cannabidiolic acid (CBA), Cannabinol (CBN) and Delta8 THCV.

Interferon λ2/3 and Interferon λ2-3 are used synonymously.

Interferon-induced antiviral effectors, interferon stimulating genes and interferon stimulated genes are used synonymously.

The term early apoptosis covers early apoptosis as a stage of apoptosis.

Apoptosis early after infection indicates timepoint when the cells undergo apoptosis after infection. This covers both early and late apoptosis as long as they happen early after infection. In the present invention it is noted that cells undergo apoptosis at 24 hrs which indicates that Cannabidiol causes Apoptosis early after infection wherein some cells may be in early apoptosis and some cells may be in late stage of apoptosis.

Patient covers any animal or human who is infected. Patients include symptomatic as well as asymptomatic patients/carrier.

Animals include pets and any other animal such as mammals and also include poultry animals such as birds raised commercially or domestically.

Procurement of Materials

Plasmids expressing ORF8 protein (YP_009724396.1) tagged at the C-terminus with 3×DYKDDDK tag (Ex-CoV229-M14), ORF10 protein (YP_009725255.1) tagged at the C-terminus with 3×hemagglutinin tag (Ex-CoV231-M07), and M protein (YP_009724393.1) tagged at the C-terminus with green fluorescent protein (Ex-CoV225-M03) were procured from GeneCopoeia (Rockland, MD, U.S.A). The control plasmid was pCMV-3Tag-3A (pCMV) and is procured from Agilent Technologies, Santa Clara, CA, U.S.A.).

CBD (#ISO60156-1) and other cannabinoids were purchased from Cedarlane Labs (Burlington, ON, Canada).

Cannabidiol has been reported previously to be effective in protecting endothelial function and integrity in human coronary artery endothelial cells (HCAECs).

Cannabidiol has reduced cytokines in induced asthma. Cannabidiol plays multiple roles such as anti-inflammatory, inhibitor of cytokines, agent to reduce LQT and a cardioprotective agent.

All Cannabinoids are considered safe. Long term treatment with Cannabidiol has been considered safe.

All viral proteins of SARS-COV-2 viz. NSP1, NSP2, NSP3, NSP4, NSP5, NSP6, NSP7, NSP8, NSP9, NSP10, NSP11, NSP12, NSP13, NSP14, NSP15, NSP16, S protein, ORF3a, E protein, M protein, ORF6, ORF7a, ORF7b, ORF8, N protein, ORF10 are being extensively researched for development of novel therapeutics to treat Covid-19 (Gordon, D. E et al, 2020).

Understanding the cellular properties and function of viral proteins will allow for testing of new therapies and strategic interventions for COVID-19. The present inventors have initiated work to elucidate mechanism of action of ORF8, ORF10, M protein and NSP5). These novel proteins, ORF8 and ORF10, have not yet been fully characterized experimentally, and their functions in cells cannot be inferred from prior work. The functions of the SARS CoV-2 form of M protein, and NSP5 are poorly understood. Indeed, little is known yet about the proteins that make up the SARS CoV-2 genome, since they all contain differences compared to other known viral proteins. Knowledge of the cellular function and pathophysiological roles of these novel proteins in the SARS CoV-2 genome is expected to provide potential new targets for therapeutic intervention. Further, the work is initiated on certain compounds which may interfere with the action of these viral proteins and prove to be useful to mankind to fight the pandemic. These compounds are those which may particularly reverse cellular perturbations caused by these viral proteins.

As on Jul. 17, 2021, the worldometers reports 190,804,868 cases of Covid-19 worldwide and 4,099,310 deaths.

Initial infection with a virus does not cause severe disease (the estimated infectious dose is 1000 virions). Illness only occurs when the virus enters cells and hijacks the cellular machinery to replicate, forming 1000s or millions of new virions.

SARS-CoV-2 has many variants, but some are of particular importance due to
i) increased transmissibility,
ii) increased virulence, and
iii) reduced effectiveness of vaccines.

Notable Variants of SARS-CoV-2 include Cluster 5, Lineage B.1.1.7, Lineage B.1.1.207, Lineage B.1.1.317, Lineage B.1.1.318, Lineage B.1.351, Lineage B.1.429/CAL.20C, Lineage B.1.525 Lineage P.1, Lineage P.3.

Mutations can arise in different genes in the SARS-CoV-2 viral genome. However, variants that arise in the spike protein are particularly concerning. Mutations causing alterations in the amino acid sequence of this viral envelope surface protein can change its structure. This may increase transmission rates by improving interactions with ACE2 (Pascarella, S., et al., 2021). More concerning, however, these mutations can also result in evasion from neutralizing antibodies produced following infection, vaccination, or the application of a therapeutic source [Lazarevic, I., et al., 2021]. Antibodies rely on a ‘key and lock’ binding mechanism that is dependent on highly specific interactions between the epitope on an antigen, such as a portion of the spike protein, and the paratope within the antibody. If the epitope changes enough, antibody recognition and interaction will be lost. The most common vaccines for SARS-CoV-2 (i.e. Astra Zeneca/Oxford, Pfizer, and Moderna) all result in the production of antibodies against the SARS-CoV-2 spike protein, based on the original wild-type strain and sequence that was circulating in 2019 [Cerutti, G., et al., 2021]. However, variants of concern (VOC) contain mutated spike proteins, and there is evidence that neutralizing antibodies generated against the sequence of the initial spike protein are less effective against new VOC [Kemp, S. A., et al., 2020].

The compositions and methods of the present invention do not restrict to any specific strain of SARS-CoV-2. The compositions and methods are effective for all known and reported strains and even future mutations. Most mutations involve mutation in one or more spike proteins. The mechanism described in this patent for SARS-CoV-2 treatment and prophylaxis occurs independent of the spike protein, and is based on enhancement of innate intracellular immunity. Thus, it would be expected to work equally well with wild-type virus or novel variants, including variants arising from mutations in the spike protein.

There are many mechanisms to attack virus. Most methods focus on inhibiting viral replication alone. One unique way to prevent the virus from replicating initially and then spreading and also from mutating, is making unavailable to the virus the infected host machinery. If the innate immune response of infected cells causes an early apoptosis causing death of infected cells, this will fragment the infected cellular machinery of the host, preventing i) replication and ii) the formation of new, infectious virions that can spread throughout the body, causing an overwhelming infection.

One problem with COVID-19 is that the innate immune response is often inadequate. Viral entry triggers interferons but if interferons are not triggered in sufficient amounts, then such inadequate induction of interferons is problematic.

Researchers have been studying prevention of viral replication through one or more means such as by measuring reduction in number of viral RNA after treatment with drugs by comparing viral RNA number in drug-treated cells and viral RNA content in vehicle-treated controls. In other studies, cells treated with drug and subjected to infection are stained for spike proteins and % of cells expressing spike proteins are plotted.

The present inventors focused on three viral proteins viz. ORF8, ORF10 and M protein.

ORF8 is an accessory protein that has been proposed to interfere with immune responses of the host. ORF8 is unique in that it may be dispensible in viral replication, but it has a unique role of evading immune surveillance of host cells i.e. it has a role in the way virus evades immunity of host cell.

Khailany et al refers to an article by Koyama et al, 2020, wherein Koyama finds that ORF10, a short 38-residue peptide from SARS-CoV-2 genome is not homologous with other proteins in the NCBI repository and Khailany further states that since ORF10 doesn't have any comparative proteins in the NCBI repository, it is one of a kind protein, which can be used to distinguish the infection more rapidly than PCR based strategies, but the further characterization of this protein is strongly required.

The membrane glycoprotein (M protein, Accession YP_009724393.1)) is a structural protein that is highly conserved across all beta-coronaviruses but has been found to have some sequence variants in the SARS CoV-2 virus, with at least 7 amino acid substitutions identified thus far (M. Bianchi et al, BioMed Research International Vol 2020 Article ID 4389089). The M protein may be important for viral entry, replication, and particle assembly within host cells, as well as for viral budding. Data from an interaction study also suggests that M protein may interfere with mitochondrial metabolism (https://doi.org/10.1038/s41586-020-2286-9) and additional cellular processes.

In an earlier filed co-pending application IN202021030633 the inventors pondered upon a question whether CBD, or other cannabinoids, can attenuate potential pro-apoptotic effects of viral proteins and concluded that the question cannot be answered unless direct investigation is done. First, whether viral proteins exhibit pro-apoptotic effects or not should be established and then whether cannabinoids alter the effect of proteins needs investigation.

When host cells are infected with virus, they transcribe interferons that will block RNA processing, to try to block viral replication. Viruses ‘hijack’ the cellular machinery to make copies of themselves, which requires RNA processing. Interferons are made as a very early response, when a virus particle enters a cell, and they shut down RNA-mediated processes in cells. This stops viruses from replicating. However, this action of interferon may also stop cells from dividing, and can cause them to undergo apoptosis, and die. However, most of the times cells selectively block viral proteins while allowing cellular proteins to continue being made.

Thus, it is essential to find factors that can augment the initial intracellular anti-viral defenses of cells, particularly those defenses which host cells can launch immediately upon viral entry such as restoring type I, II or III interferon signaling pathways.

The present invention provides i) pharmaceutical compositions and methods for treating Covid-19 infectious disease, 2) pharmaceutical compositions and methods for prophylaxis or prophylactic treatment of Covid-19 infectious disease, 3) pharmaceutical compositions and method of administering pharmaceutical compositions for preventing or reducing mutation of Sars-Cov-2 virus in a patient and 4) pharmaceutical composition and method comprising therapeutically effective amount of Cannabidiol for use in preventing or better preparing for Covid-19 infectious disease in mammals/humans who are about to get infected.

The compositions and methods of administering the compositions of the present invention produce an enhancement/augmentation of innate immunity of the patient/human due to at least one of the following effects,
a) induction of interferon transcription in the patient;
b) induction of interferon-induced antiviral effectors in the patient.

Additionally, in patients of Covid-19, compositions and methods of administering the compositions of the present invention can produce enhancement/augmentation of innate immunity of the patient as infected patient cells undergo apoptosis early after infection. As mentioned earlier, apoptosis early after infection includes both early apoptosis and late apoptosis.

The compositions of the present invention have prophylactically/therapeutically effective amount of one or more cannabinoids. A preferred cannabinoid is one or more from Cannabidiol (CBD), Cannabigerol (CBG), Cannabidiolic acid (CBA), Cannabinol (CBN) and Delta8-THCV.

The present invention includes a number of experiments carried out using HEK293 cells which were seeded in 96 or 24 well plates and transfected with plasmids expressing an empty control vector (pCMV-3Tag-3a) or vectors expressing the viral Orf8, Orf10 or M proteins.

HEK293 (human embryonic kidney) cells were chosen for transfecting with various viral proteins. HEK293 were seeded in 96 well plates, then transfected with plasmids expressing an empty control vector (pCMV-3Tag-3a) or vectors expressing the viral Orf8, Orf10 or M proteins. A few hours later the cells were treated with 1p M of the cannabinoid, then grown for 24 hours, and assayed using a colorimetric ELISA that detects BrdU incorporation.

HEK293 cells were seeded at a density of 1×10⁴ cells per well in either 96- or 24-well plates and transfected 24 hours later using JetPRIME (Polyplus Transfection, New York, NY, U.S.A.), according to the manufacturer's instructions. Briefly, for transfection in a 96-well plate, 0.1 μg of plasmid DNA and 0.25 μL jetPRIME reagent were mixed with 5 μL buffer and incubated for 10 min at room temperature. For transfection in a 24-well plate, 0.5 μg of plasmid DNA and 1.25 μL jetPRIME reagent were mixed with 50 μL buffer and incubated for 10 min at room temperature. The incubated solution was diluted in culture medium to a volume of 100 μL (for 96-well plates) or 500 μL (for 24-well plates) and the mixture replaced the culture medium of the cells. Approximately 2-3 h after transfection, cells were treated with either CBD or vehicle (0.1% ethanol) for 24 h.

Statistical analyses Two-way analysis of variance (ANOVA) followed by Tukey's post-hoc-test for multiple comparisons was performed for all experiments. Data shown are means±S.E.M.; n-values denote the number of biological replicates, which are averages of technical replicates.

The studies undertaken by the present inventors focused on following experiments:

1. Cell Proliferation Study by BrdU Incorporation

It involves to study effects of one or more cannabinoids on nuclear BrdU incorporation in cells transfected with control plasmid expressing a control vector as well as in cells transfected with plasmid expressing viral proteins;

2. Quantification of Relative Cell Numbers by Crystal Violet Staining Method

This is a simple assay useful for obtaining quantitative information about the relative density of cells adhering to multi-well cluster dishes. The cells adhering are living cells and cells washed away are dead cells. The dye in this assay, crystal violet, stains cells. Three types of cells are quantified in this assay.
i) cells transfected with the control vector;
ii) cells transfected with plasmids expressing viral proteins, and treated with vehicle;
iii) cells transfected with plasmids expressing viral proteins, and treated with one or more Cannabinoids.

Detailed process for quantification of Relative Cell number is described under example 2.

Cells transfected with the control vector shall not reduce in number. Out of the remaining two types/sets of cells, if only one show reduction in number, conclusions can be drawn.

3. Apoptosis Assay

Differences in cell number can result from changes in cell proliferation, or cell death (i.e. apoptosis or necrosis), or both. Initially both cell proliferation and apoptosis were assayed using cells treated with either vehicle or 1 μM CBD, and found a >60% increase in apoptosis indexes, but no significant effect on cell proliferation. Therefore, inventors focused their studies on apoptosis. A dose-dependent effect of one or more Cannabinoids on the activation of an early marker of apoptosis (pSIVA), and incorporation of a late marker of apoptosis (PI), was studied in cells expressing ORF8, ORF10, and M protein and also in cells transfected only with the control plasmid.

The detailed process of apoptosis assay is provided under example 3.

4. INF and ISG mRNA Expression

Interferons (IFNs) induced at different time points following infection (Lee A J and Ashkar A A) 2018 are a family of inducible cytokines with pleiotropic biological effects which help to regulate the innate, intracellular, anti-viral host defense (Durbin R K et al, 2013). There are different classes of interferons. Type I (α; alpha and β; beta) IFNs tend to slow down proliferation and regulate cell survival, Type II (γ; gamma) IFNs also regulate cell survival and proliferation, and Type III (λ; lambda) IFNs induce cell apoptosis, more so than Types I or II (Stanifer M L et al., 2019). Inadequate induction of IFNs, and especially λ-type interferons, has been identified as a factor in SARS-CoV-2 infection leading to more severe disease (Andreakos E. and Tsiodras S., 2020). The IFN λ family are important inducers of the anti-viral immune response at mucosal surfaces (Ye L et al., 2019], and people with a greater IFN λ induction tend to have less viral inflammation, and may not even develop disease (Andreakos E. and Tsiodras S., 2020)].

In the present invention, inventors worked on the hypothesis they postulated earlier which is augmented induction of interferons (INF) and interferon stimulating genes (ISG) could play a role in the enhanced apoptosis observed in cells expressing viral proteins and treated with one or more cannabinoids. In both early and late apoptosis studies, various Cannabinoids exhibited significant apoptosis in presence of viral proteins. In such studies if no significant induction of interferons is observed, these interferons have no role in apoptosis but if induction of certain interferons is significantly augmented it clearly suggests their role in the apoptosis.

Effect of one or more Cannabinoids on augmentation of certain interferon induced genes such as 2′-5′-oligoadenylate synthetase (OAS) family members in presence of viral proteins in of particular interest. Various researchers (Castelli J et al., 1998; Castelli J C et al., 1999; Flodstrom-Tullberg M et al., 2005; Stone V M et al., 2021) have previously established role of these enzymes viz. OAS family members as powerful mediators of virus-associated apoptosis. Therefore, the augmentation of OAS family members establish their role in apoptosis.

Expression of INF Genes and Expression of ISG

Inventors hypothesized that augmented induction of INF and ISG could play a role in the enhanced apoptosis observed in cells expressing viral proteins and treated with one or more Cannabinoids. This was one of the reasons for testing augmentation of interferons and interferons stimulating genes.

It has been established that interferons (IFNs) help to regulate the innate, intracellular, anti-viral host defense. Type I (u; alpha and R; beta) IFNs tend to slow down proliferation and regulate cell survival, Type II (γ; gamma) IFNs also regulate cell survival and proliferation, and Type III (k; lambda) IFNs induce cell apoptosis. Interestingly, in year 2020, Andreakos E and Tsiodras S. have shown that inadequate induction of IFNs, and especially λ-type interferons, has been identified as a factor in SARS-CoV-2 infection leading to more severe disease. Lambda-type INF are of significant interest in COVID-19 as a result of evidence showing their greater efficacy at controlling SARS-CoV-2 replication and spread (Stanifer M L et al., 2020). Among the Type III INF-stimulated genes (ISG) that act as down-stream effectors to induce apoptosis are the 2′-5′-oligoadenylate synthetase (OAS) family members (Liu M Q et al., 2012 and Palma-Ocampo H K et al., 2015) and hence study on augmentation of these members is also incorporated in the present study. Other downstream effectors included in this study are Mx1 and IFIT genes.

Methods for measuring levels of interferons and effectors gene expression are provided under example 4.

Cell Proliferation by BrdU Incorporation

BrdU is incorporated into the nucleus of dividing cells, and therefore can provide a relative measure of cell proliferation. However, since this measure is dependent on the number of cells present, measures of BrdU incorporation can only be interpreted to indicate changes in cell proliferation rate after the data are normalized to the relative number of cells that are present and being measured. Thus, a decrease in BrdU incorporation could mean that cell proliferation rates are lower, or it could mean that cell proliferation rates are not different, but that there are fewer cells being measured.

Effects of Viral proteins ORF8, ORF10, and M protein are determined on BrdU incorporation into HEK293 (human embryonic kidney) cells and further investigation is conducted to check whether treatment of transfected cells with Cannabinoid reverses any observed effect of viral protein.

It was surprisingly observed that Viral proteins did not have much impact on BrdU incorporation of HEK293 (human embryonic kidney) cells. Although no significant impact was observed, a slight reduction in BrdU incorporation rates of HEK293 (human embryonic kidney) cells was observed with all viral proteins where reduction was higher than that of a reduction where a control plasmid expressing a control vector was used. For HEK293 (human embryonic kidney) cells, even a control plasmid expressing a control vector was a foreign body, but no significant reduction in BrdU incorporation due to the control plasmid was observed.

It was further surprisingly observed that in HEK293 (human embryonic kidney) cells transfected with various viral proteins of SARS-CoV-2 when treated with one or more Cannabinoids, a sharp and significant reduction in BrdU incorporation rates of these cells were observed. This effect was common for all viral genes tested.

The inventors have run 2-way ANOVA. This has been tested in multiple separate assays on different days/weeks, with n=5 to 6 biological replicates (where separate passages of cells were considered different biological replicates). Each biological replicate was seeded in 2 to 6 technical replicates per plate, and those were averaged at each trial to give n=1 for that biological replicate in that trial. Note that these data were not normalized to the relative number of cells that were present in each well.

The BrdU incorporation level into DNA was measured by incorporating and quantifying bromodeoxyuridine (BrdU) into DNA of actively proliferating cells. The absorbance values are measured by ELISA assay with a BioTek Synergy H1 Hybrid Multi-Mode Microplate reader assay at 370 nm (reference wavelength: approx. 492 nm). FIGS. 1-5 provide results of the tests performed. FIG. 6 combines data from all figures for ready comparison. The absorbance is expressed as % untreated control and the absorbance values are normalized.

The absorbance value reflects the average quantity of BrdU incorporated into nuclei of cells in each well of cells, as DNA of these cells have incorporated bromodeoxyuridine which is measured in the assay. The absorbance when cells are transfected with a plasmid expressing an empty control vector (pCMV-3Tag-3a) is taken as a control. pCMV-3Tag-3A is a control vector that expresses a very small protein comprised of 3 FLAG tags in tandem (the amino acid sequence is DYKDDDDKDYKDDDDKDYKDDDDK. All absorbance values are to be compared to the control value. A significant deflection from the control value should reflect either reduction or enhancement in BrdU incorporation. It could reflect a difference in cell proliferation, or it could reflect a difference in cell number, indicating enhancement of cell apoptosis, which would reduce the number of cells per well.

The viral infected cells are simulated by transfecting cells with a plasmid expressing different viral proteins. Transfected cells are grown for 24 hrs in order to allow time for viral protein expression before they are assayed using a colorimetric ELISA that detects BrdU incorporation. No significant deflection but a slight reduction is observed in absorbance values, which indicates that viral proteins alone have no, or only a very small inhibitory effect on BrdU incorporation.

The effects of Cannabidiol on viral infected cells are simulated by treating cells transfected with a plasmid expressing different viral proteins with Cannabidiol. Transfected cells are grown for 24 hrs in order to allow time for viral protein expression before they are assayed using a colorimetric ELISA that detects BrdU incorporation. Surprisingly, after treatment with cannabinoids, significant reductions in absorbance are observed.

Since the absorbance when cells are transfected with a plasmid expressing an empty control vector is observed to be maximum, this absorbance value is normalized to 100% or 100 and all other values are plotted relative to the 100%.

As provided in FIG. 3, in cells expressing ORF8 protein and treated with CBD, mean BrdU incorporation was 37.28% lower than in cells expressing ORF8 protein but untreated with cannabinoids.

As provided in FIG. 4, in cells expressing Orf10 and treated with Cannabidiol, mean cell BrdU incorporation was 30.44% lower than in cells expressing Orf10 but untreated with cannabinoids.

As provided in FIG. 5, in cells expressing M protein and treated with CBD, mean cell BrdU incorporation was 37.28% lower than in cells expressing M protein but untreated with cannabinoids.

Thus, Cannabidiol impacted BrdU incorporation levels of HEK293 (human embryonic kidney) cells transfected with all three viral proteins of SARS-CoV-2. There is a significant reduction in each case. Surprisingly, Cannabidiol in untreated cells as well as in cells transfected with a control plasmid expressing a control vector does not reduce cell proliferation. This is an indication of tremendous potential of Cannabidiol in impacting either cell number or cell proliferation in infected cells.

Reduction in absorbance/reduction in BrdU incorporation after treatment with Cannabidiol does reflect a few things.
1. Interferons are produced as a response to entry of virus. However, interferons stop cell proliferation and increase apoptosis, which would reduce cell numbers and also reduce BrdU incorporation. Therefore, even when interferons are produced, it is likely to cause a reduction in absorbance. Thus, reduction in absorbance may reflect an enhanced production of interferons, and an increase in the innate intracellular response to these SARS-CoV-2 genes.
2. Reduction in BrdU incorporation and therefore absorbance can also be due to increased cell apoptosis. If reduction in absorbance is due to lower cell numbers due to increased cell apoptosis, it also reflects activation of innate cellular defense to viral genes. It is an indication that transfected cells (cells transfected with viral genes) are undergoing programmed cell death. This process has a potential where infected host cells can selectively undergo apoptosis leaving behind healthy cells.

Thus, host machinery of infected cells are destroyed or fragmented and virus has no place for replication or mutation, suppressing the creation of new variants.
3. It is possible that interferons are also produced and transfected cells are also undergoing apoptosis.

In each case, activation of cell defense after treatment with Cannabidiol is apparent.

Recently, Banerjee et al have reported that all of the viral proteins of SARS-CoV-2 responsible for inhibiting RNA processing by cells (NSP1, NSP8, NSP9, and NSP16) are produced in the first stage of the viral life cycle, prior to generation of double stranded RNA (dsRNA). dsRNA is detected by host immune sensors and a type I interferon response is triggered. This means that SARS-CoV-2 viral proteins that can stop the transcription of interferons are formed earlier than the events that trigger a type I interferon response. Therefore, unless a mechanism can allow interferons to still be produced, in the face of interferon production arrest by SARS CoV-2 proteins, then cell defenses to viral infection cannot be activated.

The present study provides such an early defense mechanism, where cells due to presence of Cannabidiol either rapidly produce interferons upon viral protein expression at viral entry or cause apoptosis of infected cells as a result of cellular defense.

The data from FIGS. 1-6 reflecting reduction in BrdU incorporation were not normalized to cell number. This may mean that reduction in BrdU incorporation as noted when cells transfected with a control plasmid and plasmid transfected with different viral proteins and treated with cannabidiol is actually not due to a reduction in the rate of cell proliferation, but to a reduction in cell number due to increased apoptosis. To confirm whether cell proliferation is reduced by treatment with Cannabidiol, cell proliferation data should be normalized to cell number.

FIGS. 7A, 7B and 7C provide BrdU incorporation/cell proliferation, where the measure of the relative incorporation of BrdU is normalized to relative cell number per well, for cells transfected with ORF8, ORF10 and M protein respectively and treated with Cannabidiol. It also provides cells transfected with a control plasmid and treated with Cannabidiol. It is noted that there is no significant difference in BrdU incorporation/cell proliferation rate when it is normalized to cell number, i.e. there is no reduction in cell proliferation rate when cells transfected with a control plasmid or with viral protein are treated with Cannabidiol. This means that treatment of cells transfected with a control plasmid or plasmid expressing viral proteins with Cannabidiol does not reduce cell proliferation rate, although reduced BrdU incorporation level was observed earlier. It is further necessary to find reasons for the earlier observed BrdU incorporation reduction. This can be done by crystal violet staining assay, which provides a relative measure of the number of adherent cells present in a well.

Quantification of Relative Cell Numbers by Crystal Violet Staining Method

FIGS. 8A, 8B, 8C respectively provide crystal violet assay where cells are stained by crystal violet and hence provide relative cell number. FIG. 8A-7-9 provides relative cell number when cells are transfected with either a control plasmid or plasmid expressing ORF8 and treated with Cannabidiol. FIG. 8B-7E provides relative cell number when cells are transfected with either a control plasmid or plasmid expressing ORF10 and treated with Cannabidiol. FIG. 7F provides relative cell number when cells are transfected with either a control plasmid or plasmid expressing M protein and treated with Cannabidiol.

FIGS. 8A, 8B, 8C respectively provide an assay where adherent cells are stained by crystal violet and hence provide a measure of the relative cell number per well. These figures show that Cannabidiol does not significantly affect the relative number of cells per well when cells only express the control plasmid. FIG. 8A-7-D provides relative cell number when cells are transfected with either a control plasmid or plasmid transfected with ORF8 and treated with or without Cannabidiol.

This figure shows that expression of ORF8 without Cannabidiol treatment does not reduce relative cell numbers, but relative cell numbers are decreased when cells expressing ORF8 and are treated with Cannabidiol, both in comparison to cells expressing ORF8 but treated with vehicle only, or in comparison to cells transfected with control plasmid and treated with Cannabidiol. This shows that Cannabidiol combines with this SARS-CoV-2 gene to cause a decrease in relative cell number that is only seen when the viral protein is combined with Cannabidiol.

FIG. 8B provides relative cell number when cells are transfected with either a control plasmid or plasmid transfected with ORF10 and treated with Cannabidiol. This figure shows that expression of ORF10 without Cannabidiol treatment does not reduce relative cell numbers, but relative cell numbers are decreased when cells express ORF10 and are treated with Cannabidiol, both in comparison to cells expressing ORF10 but treated with vehicle only, or in comparison to cells transfected with control plasmid and treated with Cannabidiol. This shows that Cannabidiol combines with this SARS-CoV-2 gene to cause a decrease in relative cell number that is only seen when the viral protein is combined with Cannabidiol.

FIG. 8C provides relative cell number when cells are transfected with either a control plasmid or plasmid transfected with M protein and treated with Cannabidiol. This figure shows that expression of M protein either with or without Cannabidiol will decrease relative cell numbers per well compared to cells transfected with the control plasmid alone and treated either with or without Cannabidiol, respectively. However, in cells expressing M-protein, Cannabidiol treatment further enhanced the reduction in relative cell number.

A dose response curve was generated by treating cells transfected with the control plasmid (pCMV) or plasmids expressing viral proteins with vehicle (0.1% EtOH) or increasing concentrations of CBD. The range of concentrations tested was based on pharmacologically achievable blood concentrations observed in human pharmacokinetic studies (Chan J Z and Duncan R E, 2021). A dose-dependent decrease in relative cell number was observed in cells expressing SARS-CoV-2 proteins, but not in cells transfected only with the control plasmid (FIG. 8D). Overall effects on relative cell number at each concentration were highly similar among the different viral proteins tested. Reduction in relative cell numbers is greater than 60% when cells transfected with plasmids expressing viral proteins are treated with 2 μM CBD. Specific analyses are shown comparing cells transfected with the control vector or plasmids expressing viral proteins, and treated with either vehicle or 2 μM CBD, which showed the greatest effect (FIG. 8E, 8F, 8G). Treatment of cells with 2 μM CBD did not significantly affect the relative number of control plasmid-transfected cells at 24 h compared to treatment with vehicle alone. In cells expressing ORF8, ORF10, or M protein and treated with vehicle alone, the relative number of cells per well was not significantly different from the number of cells in wells transfected with control plasmid. However, in cells expressing ORF8, ORF10, or M protein and treated with 2 μM CBD, there was a significant reduction in the relative cell number per well.

Crystal violet assay provides that for cells transfected with each viral protein and treated with Cannabidiol, there is a significant reduction in relative cell number. This signals at apoptosis of cells treated with Cannabidiol and necessitates apoptosis studies.

An investigation is conducted to find out the reasons whether i) reduction in cell BrdU incorporation due to treatment with Cannabidiol after transfection with viral proteins as observed earlier and provided in FIGS. 1-6 when cell BrdU incorporation data was not normalized to cell number is due to increased cell apoptosis and whether ii) reduction in relative cell number when cells are transfected with viral proteins and treated with Cannabidiol is due to increased cell apoptosis. In the present study, it has been surprisingly found that Cannabidiol, although it does not exert any significant effect on cells that are transfected with a control plasmid (empty plasmid), exerted unique and significant effects on cells that are transfected with a plasmid expressing the viral Orf8, Orf10 or M proteins. This study unfolds several avenues for use of Cannabidiol for Covid-19.

First, this reflects that the Cannabidiol may be able to differentiate and distinguish between a non-infected and infected cells and act accordingly.

Second, since cells transfected with viral proteins but untreated with Cannabidiol did not show any significant deflection/reduction from a control value, it is possible that interferons are not produced, or apoptosis is not induced in such cells.

The viral plasmids alone appear to cause only a minor decrease in cell proliferation (or, possibly increases in cell death, or both).

Early and Late Apoptosis Studies

Apoptosis Studies

Apoptotic cell death is a highly regulated process that is characterized by stereotypical and morphological changes of the cellular architecture including Cell shrinkage, plasma membrane blebbing, cell detachment, externalization of phosphatidylserine, nuclear condensation and ultimately DNA fragmentation (Taylor, R. C. et al, 2008 and Henry, C. M., 2013).

In the early apoptosis, phosphatidylserine concentration rises outside the cell. pSIVA is a marker of early apoptosis that binds to phosphatidylserine, which rises in concentration on the outside of cells when apoptosis begins, and fluoresces after binding. Cells do not yet have to be permeable in order for this interaction to happen.

In late apoptosis, Propidium iodide (PI) is used which binds to DNA, causing fluorescence. PI can only enter cells when they are in a later stage of apoptosis, where the cell and nuclear membranes have become permeable and begun to fragment, which allows PI to enter into cells. This fluorescence is read in a plate reader, which detects pSIVA and PI at different excitation/emission spectra, and so both can be present, but are read separately.

The steps are as follows:
1. HEK293 (human embryonic kidney) cells are chosen for the studies. Cells were seeded in 96-well plates at a density of 10⁴ cells per well, then grown to 60-70% confluence.
2. Then cells are either transfected with a control plasmid expressing a control vector viz. pCMV-3Tag-3A, or transfected with plasmids expressing ORF8, ORF10, or M-protein. These transfections are done in duplicate.
3. One side is treated with C-BD Cannabinoid, and one side is treated with ethanol (0.01% v/v final concentration).
3. The pORF8 and pORF10 plasmids express ORF8 (tagged with 3×FLAG tag, so pCMV-3-Tag-3A is essentially a perfect control) or ORF10 (tagged with hemagluttin), while the M-protein is tagged with green fluorescent protein. Thus, pCMV-3Tag-3A represents a control plasmid that is a small, foreign DNA, expressing a small, foreign transcript that is not viral in origin.
4. Twenty-four hours after transfection/treatment, cells were tested for relative rates of apoptosis by adding two markers of apoptosis into the medium viz. pSIVA (for early apoptosis) and propidium iodide (for late apoptosis).

The fluorescence readings gives a relative measure of the proportion of cells in a well that are in either the early stage or late stage of apoptosis at 24 hours.

The experiment is initiated with a fixed number of cells. However, when the experiment is conducted over 24 hrs., due to cell apoptosis, cells get detached and fragmented. Early and late apoptosis markers read adherent cells and hence it is necessary to take a measure of the relative number of cells in a well, when the apoptosis assay is completed.

The density of cells per well is estimated by staining cells after an assay with a cell-stain like crystal violet. Crystal violet is then eluted from cells, and the absorbance per well is measured. The higher the absorbance, the greater the number of cells.

The next step is to normalize the measures on apoptosis (i.e. total fluorescence for pSIVA and total fluorescence for PI) to relative cell number, by dividing those fluorescence values by the crystal violet absorbance measures.

FIGS. 9A and 9B provide respectively an early and late apoptosis data of HEK293 (human embryonic kidney) cells transfected with i) control plasmid expressing control vector and ii) plasmid expressing viral protein ORF8; and then treated with Cannabidiol. Cannabidiol treated cells which are transfected with control plasmid do not show any significant increase in early as well as late apoptosis but Cannabidiol treated cells which are transfected with plasmid expressing viral protein ORF8 have exhibited significant increases in early apoptosis and late apoptosis, both relative to ORF8-expressing cells treated only with vehicle control, and relative to control vector-expressing cells treated with Cannabidiol, indicating that Cannabidiol augments the cellular pro-apoptotic anti-viral response to ORF8, and this is specific to cells expressing ORF8.

FIGS. 9C and 9D provide respectively an early and late apoptosis data of HEK293 (human embryonic kidney) cells transfected with i) control plasmid expressing control vector and ii) plasmid expressing viral protein ORF10; and then treated with Cannabidiol. Cannabidiol treated cells which are transfected with control plasmid do not show any significant increase in early as well as late apoptosis but Cannabidiol treated cells which are transfected with plasmid expressing viral protein ORF10 have exhibited significant increases in early apoptosis and late apoptosis, relative to control vector-expressing cells treated with Cannabidiol, indicating that Cannabidiol augments the cellular pro-apoptotic anti-viral response to ORF10.

FIGS. 9E and 9F provide respectively an early and late apoptosis data of HEK293 (human embryonic kidney) cells transfected with i) control plasmid expressing control vector and ii) plasmid expressing viral M protein; and then treated with Cannabidiol. Cannabidiol treated cells which are transfected with control plasmid do not show any significant increase in early as well as late apoptosis but Cannabidiol treated cells which are transfected with plasmid expressing viral M protein have exhibited significant increases in early apoptosis and late apoptosis, both relative to viral M protein expressing cells treated only with vehicle control, and relative to control vector-expressing cells treated with Cannabidiol, indicating that Cannabidiol augments the cellular pro-apoptotic anti-viral response to M protein, and this is specific to cells expressing M protein.

Differences in cell number can result from changes in cell proliferation, or cell death (i.e. apoptosis or necrosis), or both. Initially both cell proliferation and apoptosis were assayed using cells treated with either vehicle or 1 μM CBD, and found a >60% increase in apoptosis indexes, but no significant effect on cell proliferation. Therefore, inventors focused their studies on apoptosis. A dose-dependent effect of CBD on the activation of an early marker of apoptosis (pSIVA), and incorporation of a late marker of apoptosis (PI), was evident in cells expressing ORF8, ORF10, and M protein, but this was not observed in cells transfected only with the control plasmid (FIGS. 9G, H). Specific analyses comparing cells transfected with the control vector or plasmids expressing viral proteins, and treated with either vehicle or 2 μM CBD (FIG. 9I-N), demonstrate several important effects.

First, this analysis shows that CBD alone, even at the highest concentration tested, does not significantly increase apoptosis in control cells. Additionally, it demonstrates that expression alone of the viral proteins ORF8, ORF10, or M protein, also does not significantly increase either early or late apoptosis relative to control cells, indicating a poor ability of cells to detect and respond to the presence of these viral RNAs or proteins. This indicates that it is only a combination of viral proteins and Cannabidiol in cells that causes apoptosis. Healthy cells (cells without viral proteins) thus will not undergo apoptosis. Interestingly, however, in cells expressing ORF8, early and late apoptosis indexes were both increased by over 6-fold in cells treated with 2 μM CBD compared to indexes in vehicle-treated cells alone (FIGS. (9I, 9L). In cells expressing ORF10 (FIGS. 9J, 9M), early and late apoptosis were increased ˜4.7- and ˜4.0-fold, respectively, while these respective increases were ˜5.6- and ˜4.7-fold in cells expressing M protein (FIGS. 9K, 9N).

Another cannabinoid, Cannabinol is tested in a similar way. A dose-dependent effect of Cannabinol on the activation of an early marker of apoptosis (pSIVA), and incorporation of a late marker of apoptosis (PI), was provided in cells expressing ORF8, ORF10, and M protein, (FIGS. 9O, 9P). Specific analyses comparing cells transfected with the control vector or plasmids expressing viral proteins, and treated with either vehicle or 1 μM Cannabinol (FIGS. 9Q-9V) provided that 1 μM cannabinol augments apoptosis above levels resulting just from viral proteins however this augmentation is significant with 2 μM Cannabinol (FIGS. 9Y-9AD).

Dose response curve for Cannabinol (FIGS. 9W and 9X) is reproduced again and same as earlier. (FIGS. 9O, 9P).

Another Cannabinoid tested is Cannabidiolic acid. Effect of Cannabidiolic acid on the activation of an early marker of apoptosis (pSIVA), and incorporation of a late marker of apoptosis (PI), was provided in cells expressing ORF8, ORF10, and M protein, (FIGS. 9AE-9AJ). Specific analyses comparing cells transfected with the control vector or plasmids expressing viral proteins, and treated with either vehicle or 1 μM Cannabinol (FIGS. 9AE-9AJ) provided that there were very few significant differences between groups expressing the same plasmid (i.e. pCMV or a viral plasmid), however, in most cases, addition of 1 uM CBDA into cells expressing a viral protein resulted in significantly greater levels of early or late apoptosis compared to cells expressing pCMV and treated with CBDA.

One more Cannabinoid tested for early and Late apoptosis effect is Cannabigerol (CBG). FIGS. 9AS and 9AT (duplicates of 9AK and 9AL) show effects of Cannabigerol on early and late apoptosis in cells expressing either the control plasmid (pCMV) or a plasmid encoding a viral protein. All these figures are dose-responses from 0 μM CBG (vehicle only) to 2 μM CBG. There are either 3 or 6 samples analyzed at each dose. The bar graphs provided in FIGS. 9AM to 9AO (early apoptosis) and FIGS. 9AP to 9AR (late apoptosis) provide comparisons for cells treated either with vehicle, or 1 μM CBG, for 24 hours. 1 μM CBG augments apoptosis above levels resulting just from viral proteins but not above control levels.

The data from these figures indicate that Cannabigerol can provide a useful treatment by inducing apoptosis when viral proteins are present. When 2 μM CBG is used, CBG treatment augmented early apoptosis in cells expressing ORF8 and M-protein as shown in FIGS. 9AU to 9AW, and late apoptosis in cells expressing M protein as shown in FIGS. 9AX to 9AZ.

Yet another Cannabinoid viz. delta 8-tetrahydrocannabivarin (d8-THCV) is also employed in the apoptosis studies. Effects of 1 μM d8-THCV on measures of early and late apoptosis (n=3) are provided in FIGS. 9BA-9BC (early apoptosis) and FIGS. 9BD-9BF (late apoptosis). In all cases, addition of 1 uM d8-THCV into cells expressing a viral protein resulted in significantly greater levels of early apoptosis compared to cells expressing pCMV and treated with d8-THCV.

Late apoptosis was significantly augmented by d8-THCV in cells expressing either pCMV or ORF8 or M-protein relative to the same cells treated only with vehicle, but the effect was not greater for cells expressing viral proteins compared to control vector.

Most of the cannabinoids tested have exhibited an ability to enhance early apoptosis or late apoptosis or both in the presence of viral proteins. Cannabidiol was the most effective and next was Cannabigerol. Nevertheless, all cannabinoids exhibited an ability to enhance apoptosis when HEK293 cells transfected with viral proteins are treated with Cannabinoids. This makes them especially useful agents in treating infectious Covid disease.

Stimulation of Interferons and Interferons Stimulated Genes/Antiviral Effectors—ORF8 Protein.

The studies involved a step of Investigation whether the effects of Cannabinoids are also due to production of interferons in cells transfected with viral proteins. This checks production of interferons and their downstream effectors upon transfection with viral proteins and upon treatment with Cannabinoids.

CBD and Interferons

The study involved estimating interferon lamda-1 levels of cells transfected with a plasmid expressing viral ORF8 and treated with Cannabidiol. The levels are also estimated in cells transfected with a control plasmid expressing control vector and treated with Cannabidiol.

FIG. 10A provides Interferon Lambda 1 mRNA levels produced when cells expressing ORF8 or a control plasmid are treated with Cannabidiol. It also provides comparison of production of Interferon Lambda 1 levels between the cells expressing ORF8, but not treated with CBD and control treated cells not treated with Cannabidiol.

In cells expressing ORF8, but not treated with CBD, Interferon Lambda 1 gene expression was not significantly elevated versus control-treated cells. This highlights the problem that cells often have an inadequate innate anti-viral response to SARS-CoV-2.

In cells expressing ORF8 and treated with CBD, CBD significantly increased expression of Interferon lambda 1 at 24 hours versus treatment with vehicle alone, indicating that CBD augments this anti-viral response to ORF8. However, CBD did not significantly affect INF lambda1 expression in cells transfected with a control plasmid, indicating that CBD specifically augments the anti-viral response to a SARS-Cov-2 gene.

Further, levels of Interferon gamma are also studied in cells transfected with control plasmid and plasmid expressing viral proteins. As provided in FIG. 10B, it provides Interferon gamma levels produced when cells expressing ORF8 or a control plasmid are treated with Cannabidiol. It provides comparison of production of Interferon gamma levels between the cells expressing ORF8, but not treated with CBD and control treated cells not treated with Cannabidiol.

CBD augmented the expression of INF-gamma in both control and ORF8-expressing cells, but had a greater effect on this expression in ORF8 expressing cells.

As provided in FIG. 10C, it provides Interferon gamma levels produced when cells expressing ORF10 or a control plasmid are treated with Cannabidiol. It provides comparison of production of Interferon gamma levels between the cells expressing ORF10, but not treated with CBD and control treated cells not treated with Cannabidiol.

FIG. 10C provides that in cells expressing ORF10 and treated with CBD, CBD significantly increased expression of Interferon gamma which is an indication of augmentation of the innate anti-viral response by cells. Expression of Interferon gamma is also seen in Cannabidiol treated cells transfected with a control plasmid, but to a lesser extent than in Cannabidiol-treated cells transfected with the SARS-CoV-2 gene ORF10.

FIGS. 10D and 10E respectively provide INF-lambda 1 and INF-lambda 2/3 levels produced when cells expressing M protein or a control plasmid are treated with Cannabidiol. They also provide comparison of INF-lambda 1 and INF-lambda 2/3 levels between the cells expressing M protein, but not treated with CBD and control treated cells not treated with Cannabidiol.

FIGS. 10D and 10E provide that Cannabidiol induced both INF-lambda 1 and INF-lambda 2/3 in cells expressing M-protein and treated with CBD, indicating that CBD augments the interferon response to this SARS-CoV-2 protein and augments this aspect of the innate intracellular anti-viral response.

This finding has immense applications. Cannabidiol is certainly augmenting the innate immune response upon transfection with viral proteins by significantly elevating levels of Interferon lambda 1 in just 24 hours.

FIGS. 10F to 10K provides effect of ORF8, ORF10, and M protein, with and without CBD, on gene expression of Type I INF. Data are means±SEM.

FIG. 10F-10H respectively provide expression of INFα in cells transfected with control plasmid (pCMV), or plasmid expressing ORF8, ORF10, M protein and treated with vehicle control (0.1% ethanol) or 2 μM CBD.

FIG. 10I-10K respectively provide expression of INFβ in cells transfected with control plasmid (pCMV), or plasmid expressing ORF8, ORF10, M protein and treated with vehicle control (0.1% ethanol) or 2 μM CBD.

Expression of the Type I INFs (INFα and INFβ) was not significantly altered by ORF8, ORF10, or M protein, either with or without 2 μM CBD (FIG. 3A-F) (FIGS. 10F-10K).

FIGS. 10L to 10T provides effect of ORF8, ORF10 and M protein, with and without CBD, on gene expression of Type II and III INF.

FIGS. 10L, 10M and 10N respectively provides expression of INFγ in cells transfected with control plasmid (pCMV) and ORF8, control plasmid (pCMV) and ORF10 and control plasmid (pCMV) and M protein and treated with vehicle control (0.10% ethanol) or 2 μM CBD (n=5).

FIG. 10M provides expression of INFγ in cells transfected with control plasmid (pCMV), ORF10 and treated with vehicle control (0.1% ethanol) or 2 μM CBD (n=5).

FIG. 10N provides expression of INFγ in cells transfected with control plasmid (pCMV), M Protein and treated with vehicle control (0.1% ethanol) or 2 μM CBD (n=5).

FIGS. 10O, 10P, 10Q respectively provide expression of INF λ1 in cells transfected with control plasmid (pCMV) and ORF8, control plasmid (pCMV) and ORF10, control plasmid (pCMV) and M protein and treated with vehicle control (0.10% ethanol) or 2 μM CBD (n=5).

FIGS. 10R, 10S, 10T respectively provide expression of INF λ2/3 in cells transfected with control plasmid (pCMV) and ORF8, control plasmid (pCMV) and ORF10, control plasmid (pCMV) and M protein and treated with vehicle control (0.10% ethanol) or 2 μM CBD (n=5).

The presence of viral proteins significantly increased gene expression of Type II INF (INFγ) and Type III INFs (INFλ1 and INF λ 2/3), and this was further augmented by 2 μM CBD (FIG. 10L-10T).

CBD and augmentation of interferons Although relative cell number and apoptosis measures were not significantly affected by 2 μM CBD, this treatment caused an ˜5-fold increase in expression of INFγ in pCMV-control cells compared to pCMV-controls cells treated only with vehicle (FIGS. 10L-10N). Similarly, INFλ1 and INFλ2/3 were increased in pCMV-transfected control cells treated with 2 μM CBD by 3-fold (FIGS. 10O-10Q) and 7-fold (FIGS. 10R-10T), respectively.

Cannabinol and Interferons

FIGS. 10U-10W respectively provide expression of INFα in cells transfected with control plasmid (pCMV), or plasmids expressing ORF8, control plasmid (pCMV), or plasmids expressing ORF10, control plasmid (pCMV), or plasmids expressing M protein and treated with vehicle control (0.1% ethanol) or 1 μM cannabinol for 24 hours.

FIGS. 10X-10Z respectively provide expression of INFβ in cells transfected with control plasmid (pCMV), or plasmids expressing ORF8, control plasmid (pCMV), or plasmids expressing ORF10, control plasmid (pCMV), or plasmids expressing M protein and treated with vehicle control (0.1% ethanol) or 1 μM cannabinol for 24 hours.

FIGS. 10U-10Z indicate that IFN alpha and IFN beta gene expression levels did not differ significantly after treatment with 1 μM Cannabinol indicating that these Type I interferons likely do not play a significant role in mediating increased apoptosis due to the combination of Cannabinol and SARS-CoV-2 proteins.

FIGS. 10AA to 10AI provides effect of ORF8, ORF10 and M protein, with and without 1 uM cannabinol, on gene expression of Type II & III IFN. Data are means±SEM, n=3.

FIGS. 10AA-10AC provide expression of INFγ in cells transfected with control plasmid (pCMV), or plasmids expressing ORF8, control plasmid (pCMV), or plasmids expressing ORF10, control plasmid (pCMV), or plasmids expressing M protein and treated with vehicle control (0.1% ethanol) or 1 μM cannabinol for 24 hours.

FIGS. 10AD-10AG provides expression of INF λ1 in cells transfected with control plasmid (pCMV), or plasmids expressing ORF8, control plasmid (pCMV), or plasmids expressing ORF10, control plasmid (pCMV), or plasmids expressing M protein and treated with vehicle control (0.1% ethanol) or 1 μM cannabinol for 24 hours.

FIGS. 10AH-10AI provide expression of INF λ 2/3 in cells transfected with control plasmid (pCMV), or plasmids expressing ORF8, control plasmid (pCMV), or plasmids expressing ORF10, control plasmid (pCMV), or plasmids expressing M protein and treated with vehicle control (0.1% ethanol) or 1 μM cannabinol for 24 hours.

Cannabinol and Augmentation of Interferons

Gene expression data was analyzed in cells treated with 1 uM cannabinol for 24 hours. Cannabinol significantly augmented the induction of IFN lambda 1 in cells expressing ORF8, ORF10, or M-protein, compared to cells expressing these proteins but treated only with vehicle, or compared to control cells transfected with pCMV and treated either with vehicle or cannabinol. A similar effect was seen for IFN lambda 2/3 in cells expressing ORF8.

Cannabigerol and Interferons

FIGS. 10AJ to 10AO provide expression of Type I INF. in cells transfected with control plasmid (pCMV), or plasmids expressing ORF8, control plasmid (pCMV), or plasmids expressing ORF10, control plasmid (pCMV), or plasmids expressing M protein and treated with vehicle control (0.1% ethanol) or 1 μM Cannabigerol (CBG) for 24 hours. Data are means±SEM, n=3

FIGS. 10AJ-10AL provide expression of INFα in cells transfected with control plasmid (pCMV), or plasmids expressing ORF8, control plasmid (pCMV), or plasmids expressing ORF10, control plasmid (pCMV), or plasmids expressing M protein and treated with vehicle control (0.1% ethanol) or 1 μM CBG for 24 hours.

FIGS. 10AM-10AO provide expression of INFβ in cells transfected with control plasmid (pCMV), or plasmids expressing ORF8, control plasmid (pCMV), or plasmids expressing ORF10, control plasmid (pCMV), or plasmids expressing M protein and treated with vehicle control (0.1% ethanol) or 1 μM CBG for 24 hours.

FIGS. 10AP to 10AX provide expression of Type II & III IFN. Data are means SEM, n=3.

FIGS. 10AP-10AR provide expression of INFγ in cells transfected with control plasmid (pCMV), or plasmids expressing ORF8, control plasmid (pCMV), or plasmids expressing ORF10, control plasmid (pCMV), or plasmids expressing M protein and treated with vehicle control (0.1% ethanol) or 1 μM CBG for 24 hours.

FIGS. 10AS-10AU provide expression of INF λ1 in cells transfected with control plasmid (pCMV), or plasmids expressing ORF8, control plasmid (pCMV), or plasmids expressing ORF10, control plasmid (pCMV), or plasmids expressing M protein and treated with vehicle control (0.1% ethanol) or 1 μM CBG for 24 hours.

FIGS. 10AV-10AX provide expression of INF λ2/3 in cells transfected with control plasmid (pCMV), or plasmids expressing ORF8, control plasmid (pCMV), or plasmids expressing ORF10, control plasmid (pCMV), or plasmids expressing M protein and treated with vehicle control (0.1% ethanol) or 1 μM CBG for 24 hours.

Cannabigerol and Augmentation of Interferons

IFN alpha and IFN beta gene expression levels did not differ significantly, after treatment with Cannabigerol indicating that these Type I interferons likely do not play a significant role in mediating increased apoptosis due to combinations of CBG and SARS-CoV-2 proteins.

Gene expression data was analyzed in cells treated with 1 μM CBG for 24 hours. CBG significantly augmented the induction of IFN gamma in cells expressing M protein, compared to cells expressing M-protein but treated only with vehicle, or compared to control cells transfected with pCMV and treated either with vehicle or CBG. This also happens for IFN-lambda 1 and ORF8, ORF10 and M protein. This means that in these cases, CBG heightened the anti-viral innate immunity response.

Cannabinoids and Interferon Stimulated Genes (ISG)

Elevation in levels of interferons is essentially an exciting finding. Interferon elevation in the human body as a response to viral entry stimulates interferon stimulated genes also called as interferon stimulated antiviral effectors. If these genes are found in a human body, it is a confirmation of body's augmented immune response and a condition where healthy individuals are better able to fight with the infection and patients are better able to handle Covid-19 infection because the situation would not worsen.

While working on such downstream effector genes, inventors came across such effector genes which are significantly elevated in cells transfected with viral protein particularly ORF8 and treated with one or more Cannabinoids.

CBD and Mx1 (Dynamin-Like GTPase Myxovirus Resistance Protein 1)

As provided in FIG. 11A, Mx1 is more highly expressed when cells transfected with ORF8 protein are treated with Cannabidiol, highlighting that Cannabidiol in combination with this SARS-CoV-2 protein augments this anti-viral response. The same effect is observed in FIG. 11F where hMx1 is highly expressed when cells transfected with ORF8 protein are treated with Cannabidiol. One more visible effect in both FIGS. 11A and 11 F is when cells transfected with a control plasmid does not express significant elevations in Mx1 or hMx when treated with Cannabidiol.

In FIG. 11B, cells transfected with both control plasmid and M protein and treated with cannabidiol have exhibited enhanced expression of Mx1. Cannabidiol induces Mx1 gene expression in cells transfected with M-protein and treated with Cannabidiol to a significantly greater extent than in cells treated with Cannabidiol but expressing only control plasmid.

FIGS. 11C, 11D and 11E respectively provide expression of Mx in cells transfected with control plasmid (pCMV) and ORF8, control plasmid (pCMV) and ORF10 and control plasmid (pCMV) and M protein treated with vehicle control (0.1% ethanol) or 2 μm CBD for 14 h (n=5).

FIG. 11F, 11G, 11H respectively provides expression of Mx in cells transfected with control plasmid (pCMV) and ORF8, control plasmid (pCMV) and ORF10 and control plasmid (pCMV) and M protein and treated with vehicle control (0.1% ethanol) or 1 μm CBD for 24 h or 48 h (n=5).

CBD and IFIT

FIG. 12A provides that cannabidiol significantly increases expression of IFIT1 either in cells transfected with M protein or control plasmid, and therefore may help to prime the innate cellular immune system to enhance ability to launch an anti-viral defense.

FIGS. 12B to 12D respectively provide effect of ORF8, ORF10, or M protein, with and without CBD, on gene expression of IFIT at 14 hrs. Data are means SEM.

FIG. 12E-12G respectively provide expression of IFIT in cells transfected with control plasmid (pCMV) and ORF8, control plasmid (pCMV) and ORF10, control plasmid (pCMV) and M protein and treated with vehicle control (0.1% ethanol) or 1 μm CBD for 48 h (n=5). In all three experiments, expression of IFIT is enhanced in cells transfected with either of ORF8, ORF10 and M protein and treated with CBD at 48 hrs.

CBD and OAS1:

FIG. 13A provides a highly significant increase in the expression of OAS1 (Oligoadenylate synthetases 1) gene in cells transfected with ORF8 protein and treated with Cannabidiol relative to all other groups and treatments.

FIG. 13B provides expression of OAS1 in cells transfected with a control plasmid or plasmid expressing ORF10 and treated with Cannabidiol. Treatment with Cannabidiol significantly increased the induction of OAS1 in cells transfected with ORF10 or control plasmid relative to treatment with vehicle alone (i.e. without Cannabidiol).

FIG. 13C provides that the cells transfected with either control plasmid or M protein and treated with Cannabidiol have exhibited significantly greater expression of OAS1 gene compared to their respective vehicle-treated cells.

As provided in FIG. 13A, a highly significant increase in the expression of the OAS1 (Oligoadenylate synthetases 1) gene is found in cells transfected with ORF8 protein and treated with Cannabidiol. The extent by which CBD augmented OAS1 in ORF8-expressing cells was significantly higher than the extent by which CBD augmented OAS1 expression in cells transfected with control vector. This finding is extremely interesting and exciting and it confirmed the role of Cannabidiol in treating Covid-19 infection.

Thus, significant enhancement in levels of OAS1 gene is a confirmation to select Cannabidiol as a therapeutic agent in Drug Development.

More interestingly, the interferon-stimulated genes are not found to be upregulated upon transfection with ORF8 alone, but only with ORF8 and Cannabidiol, although interferon gamma is significantly upregulated by transfection of cells with a plasmid expressing ORF8, even without added Cannabidiol. ORF8 is an accessory protein that has been proposed to interfere with immune responses of the host. The very protein which interferes with the immune response of the host will be unable to exert any effect in the presence of Cannabidiol because in the present case ORF8 is expressed and still, OAS1 has been produced in significant amount.

Particularly, when transfection of cells with plasmid expressing ORF8 protein is carried out without treatment with Cannabidiol, OAS1 gene expression is not significantly elevated compared to levels in vehicle-treated cells transfected with control plasmid. This indicates that an individual exposed to viral protein in absence of Cannabidiol is not able to produce interferons and interferon-induced antiviral effectors such as OAS1 in significant amounts. Also, the fact that Cannabidiol either had a lesser effect, or no effect on control-transfected cells, indicates a high margin of safety.

The data presented in FIG. 13A-13C is also very interesting because Cannabidiol in absence of viral proteins ORF8, ORF10 and M protein has also produced OAS1 (figures which means that if Cannabidiol is consumed by healthy individuals not exposed to virus they can also induce interferon transcription and interferon induced antiviral effectors, and become better prepared to respond to a viral threat.

This OAS1 expression can increase more than 10-fold, more than 20-fold and more than 30-fold when viral protein is introduced to make an individual better ready to fight against Covid-19.

FIGS. 13D to 13F provides expression of OAS1 in cells transfected with control plasmid (pCMV) and ORF8, control plasmid (pCMV) and ORF10, control plasmid (pCMV) and M protein and treated with vehicle control (0.1% ethanol) or 2 μm CBD for 14 h (n=5).

Cannabinol and OAS1 Gene Expression

FIG. 13G to 13 I respectively provide expression of OAS1 in cells transfected with control plasmid (pCMV) and ORF8, control plasmid (pCMV) and ORF10 and control plasmid (pCMV) and M protein treated with vehicle control (0.1% ethanol) or 1 μm cannabinol for 24 h (n=3).

It is seen that expression of OAS1 in cells transfected with control plasmid and treated with Cannabinol is not significantly enhanced.

Cannabigerol and OAS1 Gene Expression

FIGS. 13J, 13k and 13L respectively provide expression of OAS1 in cells transfected with control plasmid (pCMV) and ORF8, control plasmid (pCMV) and ORF10, control plasmid (pCMV) and M protein and treated with vehicle control (0.1% ethanol) or 1 μm CBG for 24 h (n=3).

In these analyses, 1 μM CBG augmented the gene expression of OAS family proteins when combined with viral proteins, above control levels, or levels seen in cells expressing the viral proteins, but not treated with 1 μM CBG.

It is seen that expression of OAS1 in cells transfected with control plasmid and treated with Cannabigerol is enhanced. This may be useful in prophylactic action of Cannabigerol or protect those who are about to get infected but not infected yet.

Cannabidiol and OAS2 Gene Expression

FIGS. 14A-14C respectively provide expression of OAS2 gene in cells transfected with control plasmid (pCMV) and ORF8, control plasmid (pCMV) and ORF10, control plasmid (pCMV) and M protein and treated with vehicle control (0.1% ethanol) or 2 μm CBD for 14 h (n=5).

It is seen from these figures that cells transfected with viral proteins and treated with Cannabidiol led to more enhanced production of OAS1.

Cannabinol and OAS2 Gene Expression

FIGS. 14D-14F respectively provide expression of OAS2 gene in cells transfected with control plasmid (pCMV) and ORF8, control plasmid (pCMV) and ORF10, control plasmid (pCMV) and M protein and treated with vehicle control (0.1% ethanol) or 2 μm CBD for 14 h (n=5).

It is seen that expression of OAS2 in cells transfected with control plasmid and treated with Cannabinol is enhanced. This may be useful in prophylactic action of Cannabinol or protect those who are about to be exposed to SARS-CoV-2 but are not infected yet.

Cannabigerol and OAS2 Gene Expression

FIGS. 14G-14I respectively provide expression of OAS2 gene in cells transfected with control plasmid (pCMV) and ORF8, control plasmid (pCMV) and ORF10, control plasmid (pCMV) and M protein and treated with vehicle control (0.1% ethanol) or 1 μm Cannabigerol for 24 h (n=3).

It is seen from these figures that cells transfected with viral proteins and treated with Cannabigerol led to more enhanced production of OAS2.

It is seen that expression of OAS2 in cells transfected with control plasmid and treated with Cannabgerol is enhanced. This may be useful in prophylactic action of Cannabigerol or protect those who are about to be exposed to SARS-CoV-2 but are not infected yet.

Cannabidiol and OAS3 Gene Expression

FIGS. 15A-15C respectively provide expression of OAS3 gene in cells transfected with control plasmid (pCMV) and ORF8, control plasmid (pCMV) and ORF10, control plasmid (pCMV) and M protein and treated with vehicle control (0.1% ethanol) or 2 μm Cannabidiol for 14 h (n=5).

It is seen from these figures that cells transfected with viral proteins and treated with Cannabidiol led to more enhanced production of OAS3.

Cannabinol and OAS3 Gene Expression

FIGS. 15D-15F respectively provide expression of OAS3 gene in cells transfected with control plasmid (pCMV) and ORF8, control plasmid (pCMV) and ORF10, control plasmid (pCMV) and M protein and treated with vehicle control (0.1% ethanol) or 1 μm Cannabinol for 24 h (n=3).

Cannabigerol and OAS3 Gene Expression

FIGS. 15G-15I respectively provide expression of OAS3 gene in cells transfected with control plasmid (pCMV) and ORF8, control plasmid (pCMV) and ORF10, control plasmid (pCMV) and M protein and treated with vehicle control (0.1% ethanol) or 1 μm Cannabigerol for 24 h (n=3).

As seen from these figures, Cannabigerol augments OAS3 genes in cells transfected with viral proteins.

Cannabidiol and OASL Gene Expression

FIGS. 16A-16C respectively provide expression of OASL gene in cells transfected with control plasmid (pCMV) and ORF8, control plasmid (pCMV) and ORF10, control plasmid (pCMV) and M protein and treated with vehicle control (0.1% ethanol) or 2 μm Cannabidiol for 14 h (n=5).

It is seen from these figures that cells transfected with viral proteins and treated with Cannabidiol led to more enhanced production of OASL.

Cannabinol and OASL Gene Expression

FIGS. 16D-16F respectively provide expression of OASL gene in cells transfected with control plasmid (pCMV) and ORF8, control plasmid (pCMV) and ORF10, control plasmid (pCMV) and M protein and treated with vehicle control (0.1% ethanol) or 1 μm Cannabinol for 24 h (n=3).

It is seen from these figures that cells transfected with viral proteins and treated with Cannabinol led to more enhanced production of OASL.

Cannabigerol and OASL Gene Expression

FIG. 16G respectively provide expression of OASL gene in cells transfected with control plasmid (pCMV) and ORF8, and treated with vehicle control (0.1% ethanol) or 1 μm Cannabinol for 24 h (n=3).

It is seen from these figures that cells transfected with viral protein and treated with Cannabigerol led to more enhanced production of OASL.

Tables 1-5 provided below summarize actions of various Cannabinoids on cells transfected with control plasmid as well as viral proteins and implications of such effects in
i) treating Covid-19 infectious disease;
ii) prophylaxis of Covid-19 infectious disease;
iii) preventing mutations of Sars-Cov-2 virus;
iv) protecting mammals and humans about to get infected from Covid-19 infectious disease.

TABLE 1
Cannabidiol

| | | | Summary of | |
|---|---|---|---|---|
| Compound | Conc'n | Outcome | relative effect | Implications |
| Cannabidiol | 1 μM | Cell | In cells expressing | (i) Infected patient - The |
| (CBD) | | number | the SARS-CoV-2 | loss of cells containing |
| | | | viral proteins | SARS-CoV-2 proteins |
| | | | ORF8, ORF10, or | helps to clear away virus |
| | | | M-protein for 24 | (ii) Prophylaxis - |
| | | | hours, cell numbers | Augmented rapid |
| | | | were decreased by | clearance of cells |
| | | | around 20%. | expressing SARS-CoV- |
| | | | However, when | 2 proteins prevents the |
| | | | cells expressed | development of |
| | | | these viral proteins | infection, and raises the |
| | | | and were also | infectious titre needed |
| | | | treated with 1 mM | (iii) Mutation |
| | | | CBD, cell number | prevention - |
| | | | decreased by | Augmented rapid |
| | | | almost 50% at 24 | clearance of cells |
| | | | hours. | expressing SARS-CoV- |
| | | | | 2 viral proteins prevents |
| | | | | viral replication |
| Cannabidiol | 2 μM | Cell | In cells expressing | (i) Infected patient - The |
| (CBD) | | number | the SARS-CoV-2 | loss of cells containing |
| | | | viral proteins | SARS-CoV-2 proteins |
| | | | ORF8, ORF10, or | helps to clear away virus |
| | | | M-protein for 24 | (ii) Prophylaxis - |
| | | | hours, cell numbers | Augmented rapid |
| | | | were decreased by | clearance of cells |
| | | | 20-25%. However, | expressing SARS-CoV- |
| | | | when cells | 2 proteins prevents the |
| | | | expressed these | development of |
| | | | viral proteins and | infection, and raises the |
| | | | were also treated | infectious titre needed |
| | | | with 2 mM CBD, | (iii) Mutation |
| | | | cell number | prevention - |
| | | | decreased by | Augmented rapid |
| | | | almost 80% at 24 | clearance of cells |
| | | | hours. Treatment of | expressing SARS-CoV- |
| | | | control cells with | 2 viral proteins prevents |
| | | | CBD did not | viral replication |
| | | | significantly | |
| | | | decrease cell | |
| | | | numbers when | |
| | | | viral proteins were | |
| | | | not present. | |
| Cannabidiol | 1 μM | Early | In cells expressing | (i) Infected patient - |
| (CBD) | | Apoptosis | the SARS-CoV-2 | Augmented early |
| | | Index | viral proteins ORF8, | apoptosis of cells |
| | | | ORF10, or M- | containing SARS-CoV- |
| | | | protein for 24 hours, | 2 proteins helps to clear |
| | | | the early apoptosis | away virus |
| | | | index was not | (ii) Prophylaxis - |
| | | | significantly higher | Augmented early |
| | | | than in cells not | apoptosis of cells |
| | | | expressing the | expressing SARS-CoV- |
| | | | proteins. However, | 2 proteins prevents the |
| | | | when cells | development of |
| | | | expressed these | infection, and raises the |
| | | | viral proteins and | infectious titre needed to |
| | | | were also treated | cause disease |
| | | | with 1 mM CBD, | (iii) Mutation |
| | | | the early apoptosis | prevention - |
| | | | index was augmented, | Augmented early |
| | | | increasing to ~2.5- | apoptosis of cells |
| | | | fold higher than in | expressing SARS-CoV- |
| | | | cells treated with | 2 viral proteins prevents |
| | | | vehicle only, at 24 | viral replication, and |
| | | | hours. Treatment of | therefore mutant |
| | | | control cells with | (variant) formation |
| | | | CBD did not | |
| | | | significantly | |
| | | | increase the early | |
| | | | apoptosis index | |
| | | | when SARS-CoV-2 | |
| | | | proteins were not | |
| | | | present, indicating | |
| | | | specificity of this | |
| | | | effect. | |
| Cannabidiol | 2 μM | Early | In cells expressing | (i) Infected patient - |
| (CBD) | | Apoptosis | the SARS-CoV-2 | Augmented early |
| | | Index | viral proteins | apoptosis of cells |
| | | | ORF8, ORF10, or | containing SARS-CoV- |
| | | | M-protein for 24 | 2 proteins helps to clear |
| | | | hours, the early | away virus |
| | | | apoptosis index | (ii) Prophylaxis - |
| | | | was not | Augmented early |
| | | | significantly higher | apoptosis of cells |
| | | | than in cells not | expressing SARS-CoV- |
| | | | expressing the | 2 proteins prevents the |
| | | | proteins. However, | development of |
| | | | when cells | infection, and raises the |
| | | | expressed these | infectious titre needed to |
| | | | viral proteins and | cause disease |
| | | | were also treated | (iii) Mutation |
| | | | with 1 mM CBD, | prevention - |
| | | | the early apoptosis | Augmented early |
| | | | index was augmented, | apoptosis of cells |
| | | | increasing to 6- to | expressing SARS-CoV- |
| | | | 8-fold higher than | 2 viral proteins prevents |
| | | | in cells treated with | viral replication, and |
| | | | vehicle only, at 24 | therefore mutant |
| | | | hours. Treatment of | (variant) formation |
| | | | control cells with | |
| | | | CBD did not | |
| | | | significantly | |
| | | | increase the early | |
| | | | apoptosis index | |
| | | | when SARS-CoV- | |
| | | | 2 proteins were not | |
| | | | present, indicating | |
| | | | specificity of this | |
| | | | effect. | |
| Cannabidiol | 1 μM | Late | In cells expressing | (i) Infected patient - |
| (CBD) | | Apoptosis | the SARS-CoV-2 | Augmented late |
| | | Index | viral proteins | apoptosis of cells |
| | | | ORF8, ORF10, or | containing SARS-CoV- |
| | | | M-protein for 24 | 2 proteins helps to clear |
| | | | hours, the late | away virus |
| | | | apoptosis index | (ii) Prophylaxis - |
| | | | was increased by | Augmented late |
| | | | approximately 25- | apoptosis of cells |
| | | | 50% (or 1.25- to | expressing SARS-CoV- |
| | | | 1.5-fold). | 2 proteins prevents the |
| | | | However, when | development of |
| | | | cells expressed | infection, and raises the |
| | | | these viral proteins | infectious titre needed to |
| | | | and were also | cause disease |
| | | | treated with 1 mM | (iii) Mutation |
| | | | CBD, the late | prevention - |
| | | | apoptosis index | Augmented late |
| | | | was augmented, | apoptosis of cells |
| | | | increasing to ~2.1- | expressing SARS-CoV- |
| | | | to 2.3-fold at 24 | 2 viral proteins prevents |
| | | | hours. Treatment of | viral replication, and |
| | | | control cells with | therefore mutant |
| | | | CBD did not | (variant) formation |
| | | | significantly | |
| | | | increase the late | |
| | | | apoptosis index | |
| | | | when viral proteins | |
| | | | were not present. | |
| Cannabidiol | 2 μM | Late | In cells expressing | (i) Infected patient - |
| (CBD) | | Apoptosis | the SARS-CoV-2 | Augmented late |
| | | Index | viral proteins | apoptosis of cells |
| | | | ORF8, ORF10, or | containing SARS-CoV- |
| | | | M-protein for 24 | 2 proteins helps to clear |
| | | | hours, the late | away virus |
| | | | apoptosis index | (ii) Prophylaxis - |
| | | | was increased by | Augmented late |
| | | | approximately 25- | apoptosis of cells |
| | | | 50% (or 1.25- to | expressing SARS-CoV- |
| | | | 1.5-fold). | 2 proteins prevents the |
| | | | However, when | development of |
| | | | cells expressed | infection, and raises the |
| | | | these viral proteins | infectious titre needed to |
| | | | and were also | cause disease |
| | | | treated with 2 mM | (iii) Mutation |
| | | | CBD, the late | prevention - |
| | | | apoptosis index | Augmented late |
| | | | was augmented, | apoptosis of cells |
| | | | increasing to ~6- to | expressing SARS-CoV- |
| | | | 7.5-fold at 24 | 2 viral proteins prevents |
| | | | hours. Treatment of | viral replication, and |
| | | | control cells with | therefore mutant |
| | | | CBD did not | (variant) formation |
| | | | significantly | |
| | | | increase the late | |
| | | | apoptosis index | |
| | | | when viral proteins | |
| | | | were not present. | |
| Cannabidiol | 2 μM | Type I | CBD does not | Modulation of Type I |
| (CBD) | | Interferon | significantly | interferon gene |
| | | gene | change the | expression does not |
| | | expression | expression of | likely contribute to the |
| | | | interferon-alpha or | augmented early and |
| | | | interferon-beta in | late apoptosis index |
| | | | either control cells, | observed in cells treated |
| | | | or cells expressing | with CBD and |
| | | | SARS-CoV-2 | expressing SARS-CoV- |
| | | | proteins after 14 | 2 proteins. |
| | | | hours. | |
| Cannabidiol | 2 μM | Type II | CBD treatment for | (i) Infected patient - |
| (CBD) | | Interferon | 14 hours | Augmented interferon |
| | | gene | significantly | gamma expression in |
| | | expression | augments the | cells containing SARS- |
| | | | expression of | CoV-2 proteins |
| | | | interferon-gamma | indicates a better innate |
| | | | in cells expressing | cellular anti-viral |
| | | | ORF8, ORF10, or | immune response to |
| | | | M-protein by 1.5- | help fight off infection |
| | | | to 3-fold relative to | in cells already infected |
| | | | vehicle treatment | with virus, including |
| | | | only. | through induction of |
| | | | | apoptosis. |
| | | | | (ii) Prophylaxis - |
| | | | | Augmented interferon |
| | | | | gamma expression in |
| | | | | cells containing SARS- |
| | | | | CoV-2 proteins |
| | | | | indicates a better innate |
| | | | | cellular anti-viral |
| | | | | immune response to |
| | | | | slow or halt viral |
| | | | | processes during initial |
| | | | | infection, helping to |
| | | | | prevent disease |
| | | | | development, and also |
| | | | | give the acquired |
| | | | | immune system more |
| | | | | time to respond |
| | | | | (iii) Mutation |
| | | | | prevention - |
| | | | | Augmented interferon |
| | | | | gamma expression in |
| | | | | cells containing SARS- |
| | | | | CoV-2 proteins |
| | | | | indicates a better innate |
| | | | | cellular anti-viral |
| | | | | immune response, |
| | | | | which slows or halts |
| | | | | viral replication, and |
| | | | | therefore limits or |
| | | | | prevents mutant |
| | | | | (variant) formation |
| Cannabidiol | 2 μM | Type II | CBD treatment for | (iv) Priming - The |
| (CBD) | | Interferon | 14 hours | induction of interferon |
| | | gene | significantly | gamma by CBD in cells |
| | | expression | augments the | that do not express |
| | | | expression of | SARS-CoV-2 proteins, |
| | | | interferon-gamma | which was not |
| | | | in cells expressing | associated with |
| | | | control plasmid | increased cell loss or |
| | | | (pCMV) by 4.8- | early or late apoptosis, |
| | | | fold relative to | indicates that cells are |
| | | | vehicle treatment | primed to be better |
| | | | only. | ready to respond to viral |
| | | | | proteins, which would |
| | | | | be protective in a |
| | | | | situation of high |
| | | | | likelihood of SARS- |
| | | | | CoV-2 exposure. |
| Cannabidiol | 2 μM | Type III | CBD treatment for | (i) Infected patient - |
| (CBD) | | Interferon | 14 hours | Augmented interferon |
| | | gene | significantly | lambda 1 and interferon |
| | | expression | augments the | lambda 2/3 expression |
| | | | expression of | in cells containing |
| | | | interferon-lambda | SARS-CoV-2 proteins |
| | | | 1 and interferon | indicates a better innate |
| | | | lambda 2/3 in cells | cellular anti-viral |
| | | | expressing ORF8, | immune response to |
| | | | ORF10, or M- | help fight off infection |
| | | | protein by | in cells already infected |
| | | | approximately 3.8- | with virus, including |
| | | | to 11.2-fold | through induction of |
| | | | relative to vehicle | apoptosis. |
| | | | treatment only. | (ii) Prophylaxis - |
| | | | | Augmented interferon |
| | | | | lambda 1 and interferon |
| | | | | lambda 2/3 expression |
| | | | | in cells containing |
| | | | | SARS-CoV-2 proteins |
| | | | | indicates a better innate |
| | | | | cellular anti-viral |
| | | | | immune response to |
| | | | | slow or halt viral |
| | | | | processes during initial |
| | | | | infection, helping to |
| | | | | prevent disease |
| | | | | development, and also |
| | | | | give the acquired |
| | | | | immune system more |
| | | | | time to respond. |
| | | | | (iii) Mutation |
| | | | | prevention - |
| | | | | Augmented interferon |
| | | | | lambda 1 and interferon |
| | | | | lambda 2/3 expression |
| | | | | in cells containing |
| | | | | SARS-CoV-2 proteins |
| | | | | indicates a better innate |
| | | | | cellular anti-viral |
| | | | | immune response, |
| | | | | which slows or halts |
| | | | | viral replication, and |
| | | | | therefore limits or |
| | | | | prevents mutant |
| | | | | (variant) formation |
| Cannabidiol | 2 μM | Type III | CBD treatment for | (iv) Priming - The |
| (CBD) | | Interferon | 14 hours | induction of interferon |
| | | gene | significantly | lambda 1 and interferon |
| | | expression | augments the | lambda 2/3 by CBD in |
| | | | expression of | cells that do not express |
| | | | interferon-lambda | SARS-CoV-2 proteins, |
| | | | 1 and interferon | which was not |
| | | | lambda 2/3 in cells | associated with |
| | | | expressing control | increased cell loss or |
| | | | plasmid (pCMV) | early or late apoptosis, |
| | | | by 3- to 7-fold | indicates that cells are |
| | | | relative to vehicle | primed to be better |
| | | | treatment only. | ready to respond to viral |
| | | | | proteins, which would |
| | | | | be protective in a |
| | | | | situation of high |
| | | | | likelihood of SARS- |
| | | | | CoV-2 exposure. |
| Cannabidiol | 2 μM | IFIT and | CBD does not | Modulation of IFIT or |
| (CBD) | | Mx gene | significantly | Mx gene expression |
| | | expression | change the | does not likely |
| | | | expression of | contribute to the |
| | | | Interferon Induced | augmented early and |
| | | | Protein with | late apoptosis index |
| | | | Tetratricopeptide | observed in cells treated |
| | | | Repeats 1 (IFIT) or | with CBD and |
| | | | Mx dynamin-like | expressing SARS-CoV- |
| | | | GTPase 1 (Mx) | 2 proteins. |
| | | | in either control | |
| | | | cells, or cells | |
| | | | expressing SARS- | |
| | | | CoV-2 proteins | |
| | | | after 14 hours. | |
| Cannabidiol | 2 μM | OAS | CBD treatment for | (i) Infected patient - |
| (CBD) | | family | 14 hours | Augmented OAS family |
| | | gene | significantly | gene expression in cells |
| | | expression | augments the | containing SARS-CoV- |
| | | | expression of 2′-5′- | 2 proteins indicates a |
| | | | oligoadenylate | better innate cellular |
| | | | synthetase | anti-viral immune |
| | | | (OAS) family | response to help fight |
| | | | members (i.e. | off infection in cells |
| | | | OAS1, OAS3, | already infected with |
| | | | OAS3, and OASL) | virus, including through |
| | | | in cells expressing | induction of apoptosis. |
| | | | ORF8, ORF10, or | (ii) Prophylaxis - |
| | | | M-protein by 3- to | Augmented OAS family |
| | | | 20-fold relative to | gene expression in cells |
| | | | vehicle treatment | containing SARS-CoV- |
| | | | only. | 2 proteins indicates a |
| | | | | better innate cellular |
| | | | | anti-viral immune |
| | | | | response to slow or halt |
| | | | | viral processes during |
| | | | | initial infection, helping |
| | | | | to prevent disease |
| | | | | development, and also |
| | | | | give the acquired |
| | | | | immune system more |
| | | | | time to respond, |
| | | | | including through |
| | | | | activation of viral RNA |
| | | | | degradation and |
| | | | | increased apoptosis of |
| | | | | infected cells. |
| | | | | (iii) Mutation |
| | | | | prevention - |
| | | | | Augmented OAS family |
| | | | | gene expression in cells |
| | | | | containing SARS-CoV- |
| | | | | 2 proteins indicates a |
| | | | | better innate cellular |
| | | | | anti-viral immune |
| | | | | response, which slows |
| | | | | or halts viral replication, |
| | | | | and therefore limits or |
| | | | | prevents mutant |
| | | | | (variant) formation, |
| | | | | including through the |
| | | | | activation of viral RNA |
| | | | | degradation. |
| Cannabidiol | 2 μM | OAS | CBD treatment for | (iv) Priming - |
| (CBD) | | family | 14 hours | induction of OAS |
| | | gene | significantly | family member gene |
| | | expression | augments the | expression by CBD in |
| | | | expression of OAS | cells that do not express |
| | | | family members in | SARS-CoV-2 proteins, |
| | | | cells expressing | which was not |
| | | | control plasmid | associated with |
| | | | (pCMV) by 2.9- to | increased cell loss or |
| | | | 7.8-fold relative to | early or late apoptosis, |
| | | | vehicle treatment | indicates that cells are |
| | | | only. | primed to be better |
| | | | | ready to respond to viral |
| | | | | proteins, which would |
| | | | | be protective in a |
| | | | | situation of high |
| | | | | likelihood of SARS- |
| | | | | CoV-2 exposure, |
| | | | | including through the |
| | | | | activation of viral RNA |
| | | | | degradation. |

TABLE 2
Cannabidiolic Acid (CBDA)

| | | | Summary of | |
|---|---|---|---|---|
| Compound | Conc'n | Outcome | relative effect | Implications |
| Cannabidiolic | 1 μM | Early | In cells | (i) Infected patient - |
| Acid | | Apoptosis | expressing the | Augmented early |
| (CBDA) | | Index | SARS-CoV-2 | apoptosis of cells |
| | | | viral proteins | containing SARS-CoV-2 |
| | | | ORF8, ORF10, | proteins helps to clear |
| | | | or M-protein for | away virus |
| | | | 24 hours, the | (ii) Prophylaxis - |
| | | | early apoptosis | Augmented early |
| | | | index was not | apoptosis of cells |
| | | | significantly | expressing SARS-CoV-2 |
| | | | different from | proteins prevents the |
| | | | control cells | development of infection, |
| | | | when grown | and raises the infectious |
| | | | only with | titre needed to cause |
| | | | vehicle control. | disease |
| | | | When cells | (iii) Mutation prevention - |
| | | | expressed these | Augmented early |
| | | | viral proteins | apoptosis of cells |
| | | | and were also | expressing SARS-CoV-2 |
| | | | treated with 1 | viral proteins prevents |
| | | | mM CBDA, the | viral replication, and |
| | | | early apoptosis | therefore mutant (variant) |
| | | | index was ~1.5- | formation |
| | | | to 2.1-fold | |
| | | | higher than in | |
| | | | control cells | |
| | | | treated with | |
| | | | CBDA. | |
| | | | Treatment of | |
| | | | control cells | |
| | | | with CBDA did | |
| | | | not significantly | |
| | | | alter the early | |
| | | | apoptosis index | |
| | | | when viral | |
| | | | proteins were | |
| | | | not present. | |
| Cannabidiolic | 1 μM | Late | In cells | (i) Infected patient - |
| Acid | | Apoptosis | expressing the | Augmented late apoptosis |
| (CBDA) | | Index | SARS-CoV-2 | of cells containing SARS- |
| | | | viral proteins | CoV-2 proteins helps to |
| | | | ORF10, or M- | clear away virus |
| | | | protein for 24 | (ii) Prophylaxis - |
| | | | hours, 1 mM | Augmented late apoptosis |
| | | | CBDA | of cells expressing SARS- |
| | | | augmented the | CoV-2 proteins prevents |
| | | | late apoptosis | the development of |
| | | | index to be 2.1- | infection, and raises the |
| | | | fold higher than | infectious titre needed to |
| | | | control cells | cause disease |
| | | | treated with 1 | (iii) Mutation prevention - |
| | | | mM CBDA. | Augmented late |
| | | | | apoptosis of cells |
| | | | | expressing SARS-CoV-2 |
| | | | | viral proteins prevents |
| | | | | viral replication, and |
| | | | | therefore mutant (variant) |
| | | | | formation. |

TABLE 3
Cannabigerol (CBG)

| | | | Summary of | |
|---|---|---|---|---|
| Compound | Conc'n | Outcome | relative effect | Implications |
| Cannabigerol | 1 mM | Early | In cells expressing | (i) Infected patient - |
| (CBG) | | Apoptosis | the SARS-CoV-2 | Augmented early |
| | | Index | viral proteins | apoptosis of cells |
| | | | ORF8, ORF10, or | containing SARS-CoV- |
| | | | M-protein for 24 | 2 proteins helps to clear |
| | | | hours, the early | away virus |
| | | | apoptosis index | (ii) Prophylaxis - |
| | | | was increased by | Augmented early |
| | | | approximately 20- | apoptosis of cells |
| | | | 30% (or 1.2- to | expressing SARS-CoV- |
| | | | 1.3-fold), which | 2 proteins prevents the |
| | | | was not | development of |
| | | | significantly | infection, and raises the |
| | | | different from | infectious titre needed to |
| | | | control cells. | cause disease |
| | | | When cells | (iii) Mutation |
| | | | expressed these | prevention - |
| | | | viral proteins and | Augmented early |
| | | | were also treated | apoptosis of cells |
| | | | with 1 mM CBG, | expressing SARS-CoV- |
| | | | the early apoptosis | 2 viral proteins prevents |
| | | | index was ~1.9- to | viral replication, and |
| | | | 2.1-fold higher | therefore mutant |
| | | | than in vehicle- | (variant) formation |
| | | | treated cells at 24 | |
| | | | hours. Treatment | |
| | | | of control cells | |
| | | | with CBG also | |
| | | | significantly | |
| | | | increased the early | |
| | | | apoptosis index | |
| | | | when viral | |
| | | | proteins were not | |
| | | | present, by 1.85- | |
| | | | fold. | |
| Cannabigerol | 2 mM | Early | In cells expressing | (i) Infected patient - |
| (CBG) | | Apoptosis | the SARS-CoV-2 | Augmented early |
| | | Index | viral proteins | apoptosis of cells |
| | | | ORF8, ORF10, or | containing SARS-CoV- |
| | | | M-protein for 24 | 2 proteins helps to clear |
| | | | hours, the early | away virus |
| | | | apoptosis index | (ii) Prophylaxis - |
| | | | was increased by | Augmented early |
| | | | approximately 20- | apoptosis of cells |
| | | | 30% (or 1.2- to | expressing SARS-CoV- |
| | | | 1.3-fold), which | 2 proteins prevents the |
| | | | was not | development of |
| | | | significantly | infection, and raises the |
| | | | different from | infectious titre needed to |
| | | | control cells. | cause disease |
| | | | When cells | (iii) Mutation |
| | | | expressed these | prevention - |
| | | | viral proteins and | Augmented early |
| | | | were also treated | apoptosis of cells |
| | | | with 2 mM CBG, | expressing SARS-CoV- |
| | | | the early apoptosis | 2 viral proteins prevents |
| | | | index was augmented | viral replication, and |
| | | | to be ~2.2- | therefore mutant |
| | | | to 3.4-fold | (variant) formation |
| | | | versus vehicle at | |
| | | | 24 hours, and was | |
| | | | greater than in | |
| | | | control cells for | |
| | | | combinations of | |
| | | | ORF8 and 2 mM | |
| | | | CBG and M- | |
| | | | protein and CBG. | |
| Cannabigerol | 1 μM | Late | In cells expressing | (i) Infected patient - |
| (CBG) | | Apoptosis | the SARS-CoV-2 | Augmented late |
| | | Index | viral proteins | apoptosis of cells |
| | | | ORF8, ORF10, or | containing SARS-CoV- |
| | | | M-protein for 24 | 2 proteins helps to clear |
| | | | hours, 1 mM CBG | away virus |
| | | | augmented the late | (ii) Prophylaxis - |
| | | | apoptosis index to | Augmented late |
| | | | be 1.5- to 2.1-fold | apoptosis of cells |
| | | | higher than in | expressing SARS-CoV- |
| | | | vehicle treated | 2 proteins prevents the |
| | | | cells. | development of |
| | | | | infection, and raises the |
| | | | | infectious titre needed to |
| | | | | cause disease |
| | | | | (iii) Mutation |
| | | | | prevention - |
| | | | | Augmented late |
| | | | | apoptosis of cells |
| | | | | expressing SARS-CoV- |
| | | | | 2 viral proteins prevents |
| | | | | viral replication, and |
| | | | | therefore mutant |
| | | | | (variant) formation |
| Cannabigerol | 2 μM | Late | In cells expressing | (i) Infected patient - |
| (CBG) | | Apoptosis | the SARS-CoV-2 | Augmented late |
| | | Index | viral proteins | apoptosis of cells |
| | | | ORF8, ORF10, or | containing SARS-CoV- |
| | | | M-protein for 24 | 2 proteins helps to clear |
| | | | hours, 1 mM CBG | away virus |
| | | | augmented the late | (ii) Prophylaxis - |
| | | | apoptosis index to | Augmented late |
| | | | be 1.7- to 2.8-fold | apoptosis of cells |
| | | | higher than in | expressing SARS-CoV- |
| | | | vehicle treated | 2 proteins prevents the |
| | | | cells. | development of |
| | | | | infection, and raises the |
| | | | | infectious titre needed to |
| | | | | cause disease |
| | | | | (iii) Mutation |
| | | | | prevention - |
| | | | | Augmented late |
| | | | | apoptosis of cells |
| | | | | expressing SARS-CoV- |
| | | | | 2 viral proteins prevents |
| | | | | viral replication, and |
| | | | | therefore mutant |
| | | | | (variant) formation |
| Cannabigerol | 1 μM | Type I | CBG does not | Modulation of Type I |
| (CBG) | | Interferon | significantly | interferon gene |
| | | gene | change the | expression does not |
| | | expression | expression of | likely contribute to the |
| | | | interferon-alpha | augmented early and |
| | | | or interferon-beta | late apoptosis index |
| | | | in either control | observed in cells treated |
| | | | cells, or cells | with CBG and |
| | | | expressing SARS- | expressing SARS-CoV- |
| | | | CoV-2 proteins | 2 proteins. |
| | | | after 24 hours. | |
| Cannabigerol | 1 μM | Type II | CBG treatment for | (i) Infected patient - |
| (CBG) | | Interferon | 24 hours | Augmented interferon |
| | | gene | significantly | gamma expression in |
| | | expression | augments the | cells containing SARS- |
| | | | expression of | CoV-2 proteins |
| | | | interferon-gamma | indicates a better innate |
| | | | in cells expressing | cellular anti-viral |
| | | | M-protein by 3.6- | immune response to |
| | | | fold relative to | help fight off infection |
| | | | vehicle treatment | in cells already infected |
| | | | only. | with virus, including |
| | | | | through induction of |
| | | | | apoptosis. |
| | | | | (ii) Prophylaxis - |
| | | | | Augmented interferon |
| | | | | gamma expression in |
| | | | | cells containing SARS- |
| | | | | CoV-2 proteins |
| | | | | indicates a better innate |
| | | | | cellular anti-viral |
| | | | | immune response to |
| | | | | slow or halt viral |
| | | | | processes during initial |
| | | | | infection, helping to |
| | | | | prevent disease |
| | | | | development, and also |
| | | | | give the acquired |
| | | | | immune system more |
| | | | | time to respond |
| | | | | (iii) Mutation |
| | | | | prevention - |
| | | | | Augmented interferon |
| | | | | gamma expression in cells |
| | | | | containing SARS-CoV- |
| | | | | 2 proteins indicates a |
| | | | | better innate cellular |
| | | | | anti-viral immune |
| | | | | response, which slows |
| | | | | or halts viral replication, |
| | | | | and therefore limits or |
| | | | | prevents mutant |
| | | | | (variant) formation |
| Cannabigerol | 1 μM | Type III | CBG treatment for | (i) Infected patient - |
| (CBG) | | Interferon | 24 hours | Augmented interferon |
| | | gene | significantly | lambda 1 in cells |
| | | expression | augments the | containing SARS-CoV- |
| | | | expression of | 2 proteins indicates a |
| | | | interferon-lambda | better innate cellular |
| | | | 1 in cells | anti-viral immune |
| | | | expressing ORF8, | response to help fight |
| | | | or ORF10, by | off infection in cells |
| | | | approximately 10- | already infected with |
| | | | to 12-fold relative | virus, including through |
| | | | to vehicle | induction of apoptosis. |
| | | | treatment only. | (ii) Prophylaxis - |
| | | | Interferon lambda | Augmented interferon |
| | | | 2/3 was not | lambda 1 expression in |
| | | | significantly | cells containing SARS- |
| | | | induced by | CoV-2 proteins |
| | | | expression of viral | indicates a better innate |
| | | | proteins or CBG, | cellular anti-viral |
| | | | indicating that | immune response to |
| | | | these genes likely | slow or halt viral |
| | | | do not play a role | processes during initial |
| | | | in augmented | infection, helping to |
| | | | apoptosis | prevent disease |
| | | | observed with | development, and also |
| | | | CBG treatment. | give the acquired |
| | | | | immune system more |
| | | | | time to respond. |
| | | | | (iii) Mutation |
| | | | | prevention - |
| | | | | Augmented interferon |
| | | | | lambda 1 in cells |
| | | | | containing SARS-CoV- |
| | | | | 2 proteins indicates a |
| | | | | better innate cellular |
| | | | | anti-viral immune |
| | | | | response, which slows |
| | | | | or halts viral replication, |
| | | | | and therefore limits or |
| | | | | prevents mutant |
| | | | | (variant) formation |
| Cannabigerol | 1 μM | OAS | CBG treatment for | (i) Infected patient - |
| (CBG) | | family | 24 hours | Augmented OAS family |
| | | gene | significantly | gene expression in cells |
| | | expression | augments the | containing SARS-CoV- |
| | | | expression of 2'- | 2 proteins indicates a |
| | | | 5'-oligoadenylate | better innate cellular |
| | | | synthetase | anti-viral immune |
| | | | (OAS) family | response to help fight |
| | | | members (i.e | off infection in cells |
| | | | OAS1, OAS3, | already infected with |
| | | | OAS3, and | virus, including through |
| | | | OASL) in cells | induction of apoptosis. |
| | | | expressing ORF8, | (ii) Prophylaxis - |
| | | | ORF10, or M- | Augmented OAS family |
| | | | protein by 1.3- to | gene expression in cells |
| | | | 255-fold relative | containing SARS-CoV- |
| | | | to vehicle | 2 proteins indicates a |
| | | | treatment only. | better innate cellular |
| | | | | anti-viral immune |
| | | | | response to slow or halt |
| | | | | viral processes during |
| | | | | initial infection, helping |
| | | | | to prevent disease |
| | | | | development, and also |
| | | | | give the acquired |
| | | | | immune system more |
| | | | | time to respond, |
| | | | | including through |
| | | | | activation of viral RNA |
| | | | | degradation and |
| | | | | increased apoptosis of |
| | | | | infected cells. |
| | | | | (iii) Mutation |
| | | | | prevention - |
| | | | | Augmented OAS family |
| | | | | gene expression in cells |
| | | | | containing SARS-CoV- |
| | | | | 2 proteins indicates a |
| | | | | better innate cellular |
| | | | | anti-viral immune |
| | | | | response, which slows |
| | | | | or halts viral replication, |
| | | | | and therefore limits or |
| | | | | prevents mutant |
| | | | | (variant) formation, |
| | | | | including through the |
| | | | | activation of viral RNA |
| | | | | degradation. |
| Cannabigerol | 1 μM | OAS | CBG treatment for | (iv) Priming - The |
| (CBG) | | family | 24 hours | induction of OAS2 |
| | | gene | significantly | expression by CBG in |
| | | expression | augments the | cells that do not express |
| | | | expression of | SARS-CoV-2 proteins, |
| | | | OAS2 in cells | which was not |
| | | | expressing control | associated with |
| | | | plasmid (pCMV) | increased cell loss or |
| | | | by 14-fold relative | early or late apoptosis, |
| | | | to vehicle | indicates that cells are |
| | | | treatment only. | primed to be better |
| | | | | ready to respond to viral |
| | | | | proteins, which would |
| | | | | be protective in a |
| | | | | situation of high |
| | | | | likelihood of SARS- |
| | | | | CoV-2 exposure, |
| | | | | including through the |
| | | | | activation of viral RNA |
| | | | | degradation. |

TABLE 4
Cannabinol

| | | | Summary of relative | |
|---|---|---|---|---|
| Compound | Conc'n | Outcome | effect | Implications |
| Cannabinol | 1 μM | Early | In cells expressing | (i) Infected patient - |
| (CBN) | | Apoptosis | the SARS-CoV-2 | Augmented early |
| | | Index | viral proteins ORF8, | apoptosis of cells |
| | | | ORF10, or M- | containing SARS-CoV-2 |
| | | | protein for 24 hours, | proteins helps to clear |
| | | | the early apoptosis | away virus |
| | | | index was not | (ii) Prophylaxis - |
| | | | significantly | Augmented early |
| | | | different from | apoptosis of cells |
| | | | control cells. When | expressing SARS-CoV- |
| | | | cells expressed these | 2 proteins prevents the |
| | | | viral proteins and | development of |
| | | | were also treated | infection, and raises the |
| | | | with 1 mM CBN, the | infectious titre needed to |
| | | | early apoptosis | cause disease |
| | | | index was ~1.8- to | (iii) Mutation prevention - |
| | | | 1.9-fold higher than | Augmented early |
| | | | in vehicle-treated | apoptosis of cells |
| | | | cells at 24 hours. | expressing SARS-CoV- |
| | | | Treatment of control | 2 viral proteins prevents |
| | | | cells with CBN also | viral replication, and |
| | | | significantly | therefore mutant |
| | | | increased the early | (variant) formation |
| | | | apoptosis index | |
| | | | when viral proteins | |
| | | | were not present, by | |
| | | | 1.82-fold. | |
| Cannabinol | 2 μM | Early | In cells expressing | (i) Infected patient - |
| (CBN) | | Apoptosis | the SARS-CoV-2 | Augmented early |
| | | Index | viral proteins ORF8, | apoptosis of cells |
| | | | ORF10, or M- | containing SARS-CoV-2 |
| | | | protein for 24 hours, | proteins helps to clear |
| | | | the early apoptosis | away virus |
| | | | index was not | (ii) Prophylaxis - |
| | | | significantly | Augmented early |
| | | | different from | apoptosis of cells |
| | | | control cells. When | expressing SARS-CoV- |
| | | | cells expressed these | 2 proteins prevents the |
| | | | viral proteins and | development of |
| | | | were also treated | infection, and raises the |
| | | | with 2 mM CBN, the | infectious titre needed to |
| | | | early apoptosis | cause disease |
| | | | index was | |
| | | | augmented to be ~1.8- | (iii) Mutation prevention - |
| | | | to 2.7-fold | Augmented early |
| | | | greater than cells | apoptosis of cells |
| | | | treated with vehicle | expressing SARS-CoV- |
| | | | at 24 hours, and was | 2 viral proteins prevents |
| | | | greater than in | viral replication, and |
| | | | control cells for | therefore mutant |
| | | | combinations of | (variant) formation |
| | | | ORF8 and 2 mM | |
| | | | CBN and M-protein | |
| | | | and CBN. | |
| Cannabinol | 1 μM | Late | In cells expressing | (i) Infected patient - |
| (CBN) | | Apoptosis | the SARS-CoV-2 | Augmented late |
| | | Index | viral proteins ORF8, | apoptosis of cells |
| | | | ORF10, or M- | containing SARS-CoV-2 |
| | | | protein for 24 hours, | proteins helps to clear |
| | | | 1 mM CBN | away virus |
| | | | augmented the late | (ii) Prophylaxis - |
| | | | apoptosis index to be | Augmented late |
| | | | 1.7- to 1.8-fold | apoptosis of cells |
| | | | higher than in | expressing SARS-CoV- |
| | | | vehicle treated cells. | 2 proteins prevents the |
| | | | The late apoptosis | development of |
| | | | index was | infection, and raises the |
| | | | augmented by 1 mM | infectious titre needed to |
| | | | CBN in cells | cause disease |
| | | | expressing ORF10 | (iii) Mutation prevention - |
| | | | compared to control | Augmented late |
| | | | cells treated with 1 | apoptosis of cells |
| | | | mM CBN. | expressing SARS-CoV- |
| | | | | 2 viral proteins prevents |
| | | | | viral replication, and |
| | | | | therefore mutant |
| | | | | (variant) formation |
| Cannabinol | 2 μM | Late | In cells expressing | (i) Infected patient - |
| (CBN) | | Apoptosis | the SARS-CoV-2 | Augmented late |
| | | Index | viral proteins ORF8, | apoptosis of cells |
| | | | ORF10, or M- | containing SARS-CoV-2 |
| | | | protein for 24 hours, | proteins helps to clear |
| | | | 1 mM CBN | away virus |
| | | | augmented the late | (ii) Prophylaxis - |
| | | | apoptosis index to be | Augmented late |
| | | | 1.6- to 1.9-fold | apoptosis of cells |
| | | | higher than in | expressing SARS-CoV- |
| | | | vehicle treated cells. | 2 proteins prevents the |
| | | | CBN augmented | development of |
| | | | apoptosis above | infection, and raises the |
| | | | control cells levels | infectious titre needed to |
| | | | in cells expressing a | cause disease |
| | | | combination of M- | (iii) Mutation prevention - |
| | | | protein with 2 mM | Augmented late |
| | | | CBN. | apoptosis of cells |
| | | | | expressing SARS-CoV- |
| | | | | 2 viral proteins prevents |
| | | | | viral replication, and |
| | | | | therefore mutant |
| | | | | (variant) formation |
| Cannabinol | 1 μM | Type I | CBN does not | Modulation of Type I |
| (CBN) | | Interferon | significantly change | interferon gene |
| | | gene | the expression of | expression does not |
| | | expression | interferon-alpha or | likely contribute to the |
| | | | interferon-beta in | augmented early and late |
| | | | either control cells, | apoptosis index |
| | | | or cells expressing | observed in cells treated |
| | | | SARS-CoV-2 | with CBN and |
| | | | proteins after 24 | expressing SARS-CoV- |
| | | | hours. | 2 proteins. |
| Cannabinol | 1 μM | Type II | CBN treatment for | Modulation of Type II |
| (CBN) | | Interferon | 24 hours does not | interferon gamma gene |
| | | gene | significantly | expression does not |
| | | expression | augment the | likely contribute to the |
| | | | expression of | augmented early and late |
| | | | interferon-gamma in | apoptosis index |
| | | | cells expressing | observed in cells treated |
| | | | SARS-CoV-2 | with CBN and |
| | | | proteins. | expressing SARS-CoV- |
| | | | | 2 proteins. |
| Cannabinol | 1 μM | Type | CBN treatment for | (i) Infected patient - |
| (CBN) | | III | 24 hours | Augmented interferon |
| | | Interferon | significantly | lambda 1 or interferon |
| | | gene | augments the | lambda 2/3 in cells |
| | | expression | expression of | containing SARS-CoV-2 |
| | | | interferon-lambda 1 | proteins indicates a |
| | | | in cells expressing | better innate cellular |
| | | | ORF8, ORF10, or | anti-viral immune |
| | | | M-protein by | response to help fight off |
| | | | approximately 3- to | infection in cells already |
| | | | 13-fold relative to | infected with virus, |
| | | | vehicle treatment | including through |
| | | | only. Interferon | induction of apoptosis. |
| | | | lambda 2/3 gene | (ii) Prophylaxis - |
| | | | expression was | Augmented interferon |
| | | | augmented only in | lambda 1 or interferon |
| | | | cells expressing | lambda 2/3 expression in |
| | | | ORF8 with 1 mM | cells containing SARS- |
| | | | CBN treatment. | CoV-2 proteins indicates |
| | | | | a better innate cellular |
| | | | | anti-viral immune |
| | | | | response to slow or halt |
| | | | | viral processes during |
| | | | | initial infection, helping |
| | | | | to prevent disease |
| | | | | development, and also |
| | | | | give the acquired |
| | | | | immune system more |
| | | | | time to respond. |
| | | | | (iii) Mutation prevention - |
| | | | | Augmented interferon |
| | | | | lambda 1 or interferon |
| | | | | lambda 2/3in cells |
| | | | | containing SARS-CoV-2 |
| | | | | proteins indicates a |
| | | | | better innate cellular |
| | | | | anti-viral immune |
| | | | | response, which slows or |
| | | | | halts viral replication, |
| | | | | and therefore limits or |
| | | | | prevents mutant |
| | | | | (variant) formation |
| Cannabinol | 1 μM | Type | CBN treatment for | (iv) Priming - The |
| (CBN) | | III | 24 hours | induction of interferon |
| | | Interferon | significantly | lambda 1 and interferon |
| | | gene | augments the | lambda 2/3 by CBN in |
| | | expression | expression of | cells that do not express |
| | | | interferon-lambda 1 | SARS-CoV-2 proteins, |
| | | | and interferon | which was not associated |
| | | | lambda 2/3 in cells | with increased cell loss |
| | | | expressing control | or early or late apoptosis, |
| | | | plasmid (pCMV) by | indicates that cells are |
| | | | 3.4- to 35-fold | primed to be better ready |
| | | | relative to vehicle | to respond to viral |
| | | | treatment only. | proteins, which would be |
| | | | | protective in a situation |
| | | | | of high likelihood of |
| | | | | SARS-CoV-2 exposure. |
| Cannabinol | 1 μM | OAS | CBN treatment for | (i) Infected patient - |
| (CBN) | | family | 24 hours | Augmented OAS family |
| | | gene | significantly | gene expression in cells |
| | | expression | augments the | containing SARS-CoV-2 |
| | | | expression of 2′-5′- | proteins indicates a |
| | | | oligoadenylate | better innate cellular |
| | | | synthetase | anti-viral immune |
| | | | (OAS) family | response to help fight off |
| | | | members (i.e. | infection in cells already |
| | | | OAS2, OAS3, and | infected with virus, |
| | | | OASL) in cells | including through |
| | | | expressing ORF8, | induction of apoptosis. |
| | | | ORF10, or M- | (ii) Prophylaxis - |
| | | | protein by 2- to 39- | Augmented OAS family |
| | | | fold relative to | gene expression in cells |
| | | | vehicle treatment | containing SARS-CoV-2 |
| | | | only. | proteins indicates a |
| | | | | better innate cellular |
| | | | | anti-viral immune |
| | | | | response to slow or halt |
| | | | | viral processes during |
| | | | | initial infection, helping |
| | | | | to prevent disease |
| | | | | development, and also |
| | | | | give the acquired |
| | | | | immune system more |
| | | | | time to respond, |
| | | | | including through |
| | | | | activation of viral RNA |
| | | | | degradation and |
| | | | | increased apoptosis of |
| | | | | infected cells. |
| | | | | (iii) Mutation prevention - |
| | | | | Augmented OAS |
| | | | | family gene expression |
| | | | | in cells containing |
| | | | | SARS-CoV-2 proteins |
| | | | | indicates a better innate |
| | | | | cellular anti-viral |
| | | | | immune response, which |
| | | | | slows or halts viral |
| | | | | replication, and therefore |
| | | | | limits or prevents mutant |
| | | | | (variant) formation, |
| | | | | including through the |
| | | | | activation of viral RNA |
| | | | | degradation. |
| Cannabinol | 1 μM | OAS | CBN treatment for | (iv) Priming - The |
| (CBN) | | family | 24 hours | induction of OAS2 |
| | | gene | significantly | expression by CBN in |
| | | expression | augments the | cells that do not express |
| | | | expression of OAS1, | SARS-CoV-2 proteins, |
| | | | OAS2, OAS3, and | which was not associated |
| | | | OASL in cells | with increased cell loss |
| | | | expressing control | or early or late apoptosis, |
| | | | plasmid (pCMV) by | indicates that cells are |
| | | | 7.5- to 169-fold | primed to be better ready |
| | | | relative to vehicle | to respond to viral |
| | | | treatment only. | proteins, which would be |
| | | | | protective in a situation |
| | | | | of high likelihood of |
| | | | | SARS-CoV-2 exposure, |
| | | | | including through the |
| | | | | activation of viral RNA |
| | | | | degradation. |

TABLE 5
Delta 8-tetrahydro-cannabiverin (d8-THCV)

| | | | Summaryof | |
|---|---|---|---|---|
| Compound | Conc'n | Outcome | relative effect | Implications |
| Delta 8- | 1 μM | Early | In cells | (i) Infected patient - |
| tetrahydro- | | Apoptosis | expressing the | Augmented early |
| cannabiverin | | Index | SARS-CoV-2 | apoptosis of cells |
| (d8-THCV) | | | viral proteins | containing SARS-CoV-2 |
| | | | ORF8, ORF10, or | proteins helps to clear |
| | | | M-protein for 24 | away virus |
| | | | hours, the early | (ii) Prophylaxis - |
| | | | apoptosis index | Augmented early |
| | | | was not | apoptosis of cells |
| | | | significantly | expressing SARS-CoV-2 |
| | | | different from | proteins prevents the |
| | | | control cells when | development of |
| | | | cells were treated | infection, and raises the |
| | | | only with vehicle | infectious titre needed to |
| | | | control. When | cause disease |
| | | | cells expressed | (iii) Mutation prevention - |
| | | | these viral | Augmented early |
| | | | proteins and were | apoptosis of cells |
| | | | also treated with 1 | expressing SARS-CoV-2 |
| | | | mM d8-THCV, | viral proteins prevents |
| | | | the early | viral replication, and |
| | | | apoptosis index | therefore mutant |
| | | | was ~1.4- to 1.5- | (variant) formation |
| | | | fold higher than in | |
| | | | control cells | |
| | | | treated with d8- | |
| | | | THCV. In cells | |
| | | | expressing | |
| | | | ORF10 or M- | |
| | | | protein and | |
| | | | treated with 1 mM | |
| | | | d8-THCV, the | |
| | | | early apoptosis | |
| | | | index was | |
| | | | augmented | |
| | | | compared to cells | |
| | | | expressing these | |
| | | | viral proteins but | |
| | | | treated only with | |
| | | | vehicle. | |
| | | | Treatment of | |
| | | | control cells with | |
| | | | d8-THCV did not | |
| | | | significantly alter | |
| | | | the early | |
| | | | apoptosis index | |
| | | | when viral | |
| | | | proteins were not | |
| | | | present. | |
| Delta 8- | 1 μM | Late | In cells | (i) Infected patient - |
| tetrahydro- | | Apoptosis | expressing the | Augmented late |
| cannabiverin | | Index | SARS-CoV-2 | apoptosis of cells |
| (d8-THCV) | | | viral proteins | containing SARS-CoV-2 |
| | | | ORF8 or M- | proteins helps to clear |
| | | | protein for 24 | away virus |
| | | | hours, 1 mM d8- | (ii) Prophylaxis - |
| | | | THCV | Augmented late |
| | | | augmented the | apoptosis of cells |
| | | | late apoptosis | expressing SARS-CoV-2 |
| | | | index to be 1.4- to | proteins prevents the |
| | | | 1.8-fold higher | development of |
| | | | than cells treated | infection, and raises the |
| | | | with vehicle only. | infectious titre needed to |
| | | | | cause disease |
| | | | | (iii) Mutation prevention - |
| | | | | Augmented late |
| | | | | apoptosis of cells |
| | | | | expressing SARS-CoV-2 |
| | | | | viral proteins prevents |
| | | | | viral replication, and |
| | | | | therefore mutant |
| | | | | (variant) formation |

### Results of 1 μM Cannabidiol

1) Stimulation of Interferons and Interferon-Stimulated Antiviral Effectors—ORF10 Protein

As provided in FIG. 13, in cells expressing ORF10, CBD significantly increased expression of Interferon gamma which is an indication of augmentation of immunity. Expression of Interferon gamma is also seen in Cannabidiol treated cells transfected with a control plasmid. This expression in the absence of viral protein increases 3-4 folds in presence of viral protein ORF10. Thus, Cannabidiol significantly augments the innate immune response in cells expressing ORF10.

CBD significantly augmented the induction of OAS1 in response to ORF10, compared to cells treated only with vehicle. The OAS1 induction in response to ORF10 plus CBD was lower than the induction in response to CBD plus control plasmid. Nevertheless, CBD did augment this anti-viral response in cells transfected with either plasmid.

2) Stimulation of Interferons and Interferons Stimulated Antiviral Effectors—M Protein

Cannabidiol induced both INF-lambda 1 and INF-lambda 2/3 in cells expressing M-protein, indicating that Cannabidiol augments the interferon response to this SARS-CoV-2 protein and augments the innate immune response. Cannabidiol did not cause an induction of INF-lambda 1, or interferons lambda 2/3 in cells transfected only with control plasmid.

Mx1 is another interferon induced anti-viral effector. Cells transfected with M protein and treated with Cannabidiol have higher expression of Mx1 than cells transfected with control vector and treated with Cannabidiol. This indicates a potential that CBD ‘primes’ cells to be ready to respond to a viral threat, upon expression of viral genes. CBD treatment led to enhanced expression of Mx1 in cells expressing M-protein compared to cells overexpressing control plasmid, indicating an enhanced anti-viral response in the presence of this viral gene.

In yet one more interesting study, cells transfected with either control plasmid or M protein and treated with cannabidiol have exhibited significant elevations in expression of OAS1 gene relative to vehicle-treated control cells.

Cannabidiol augments interferons and interferon-induced anti-viral effectors even in the absence of viral proteins. This is a strong reason to select Cannabidiol as a prophylactic medicine where CBD may help to prime this aspect of the innate immune response. IFIT1 (interferon-induced protein with tetratricopeptide repeats) is another interferon-induced anti-viral effector. As provided in FIG. 19, Cells transfected with both control plasmid and M protein and treated with cannabidiol have exhibited elevated expression of IFIT1 relative to cells treated with vehicle alone. This augmentation was lower in the cells expressing M-protein than in cells expressing control plasmid, but it was still a significant augmentation, indicating that CBD may help to prime this aspect of the innate immune response.

As reported by Zhou Sirui et al (Zhou Sirui et al, 2021), for treating Covid-19, “Available pharmacological agents that increase OAS1 levels could be prioritized for drug development”. Out of the three viral proteins tested, two have shown a strong reason to select Cannabidiol for Covid-19 therapy.

Surprisingly, the inventors of the present invention came across various facts that reinforce multiple roles of Cannabidiol in prophylaxis and treatment of Covid-19.

These roles are summarized below:

3) Role in Apoptosis
1. CBD augments early and late apoptosis induction, 24 hours after cells are transfected with viral genes, which suggests that CBD can help cells to fight off an initial infection. Infected host cells apoptose and host machinery is not available for viral replication and mutation. The induction of apoptosis early after viral transcripts appear in a cell is highly protective against infection. Viruses enter cells, and then ‘hijack’ the cellular machinery to begin producing new virus. This is what results in a wide-spread infection, and it is also the time when mutations can be introduced to the viral genome (i.e. during replication of the viral genome), resulting in the emergence of new variants. However, apoptosis causes fragmentation of the cellular organelles, and eventually the cell. If it happens early after infection, it can prevent infected cells from making new virus. This would result in early elimination of virus and infected cells from a person, who would likely not even realize they had been infected.
2. Early apoptosis in cells expressing ORF8 is highly significant as this very protein is proposed to interfere with immune responses of host. ORF8 is unique in that it may be dispensible in viral replication but it has a unique role of evading immune surveillance of host cells i.e. it has a role in the way virus evades immunity of host cells.
3. CBD does not increase early or late apoptosis in control transfected cells versus vehicle alone, indicating a high degree of safety of CBD and that effects of the combination of CBD and SARS-CoV-2 viral proteins are specific.
4. Induction of interferons results in an innate, intracellular, anti-viral host defense that does not require immune cells, per se. There are different types of interferons. Type 1 (alpha and beta) tend to slow down proliferation, and also regulate cell survival. Type II (gamma) tends to also regulate both cell survival and proliferation. Type III interferons (i.e. Lambda-type interferons) tend to force cells towards apoptosis, much more so than Type I or II. Although not much change is observed in Type I interferons, some significant increase in Type II and also in Type III interferons are noted as a result of CBD treatment. CBD played dual roles. It expressed interferons in cells transfected with viral proteins but also in cells transfected with a control plasmid and treated with Cannabidiol. Thus it is seen that CBD prepares host for viral threats even in the absence of virus.
5. Some of the interferon induced anti-viral effectors that are expressed during treatment with Cannabidiol include Mx1, IFIT1 and OAS1.
6. IFIT is short for ‘interferon-induced protein with tetratricopeptide repeats’. It binds to RNAs that lack a signature methylation sequence (indicating foreign (and possibly viral) origin) to inhibit their translation—and therefore is an innate cellular mechanism that functions to help stop viral mRNA from being translated into protein. It also “interacts with other cellular proteins to expand their contribution to regulation of the host antiviral response by modulating innate immune signaling and apoptosis.” Inducing IFIT, therefore, should help to slow viral replication. CBD enhanced IFIT1 transcription in control-transfected cells, and in cells expressing M-protein.
7. MX1 (Dynamin-Like GTPase myxovirus resistance protein 1) is an interferon-stimulated gene. This gene can be induced by IFN type I and/or type III (i.e. INF lambda). Mx1 inhibits the transcription of viral RNA. Bizzotto Juan et al (Bizzotto Juan et al, 2020) reports that MX1 levels increase with increasing viral load in SARS CoV-2 infection. Mx1 transcription is enhanced by a combination of CBD and ORF8 or CBD and M-protein.
https://pubmed.ncbi.nlm.nih.gov/32989429/
8. OAS1 stands for Oligoadenylate synthetases (OAS), which are a family of interferon-stimulated genes that can induce RNA degradation in virus by activating RNaseL.

Zhou et al have reported that higher levels of OAS1 are associated with a Neanderthal SNP in people of European ancestry, and higher levels reduce the risk of COVID-19 death, ventilation, hospitalization or susceptibility.

Out of the three proteins tested, cells transfected with two proteins namely ORF8 and M protein and treated with Cannabidiol exhibited significantly increased expression of the OAS1 gene. This makes Cannabidiol a confirmed candidate for treating Covid-19.

OAS1 when produced will activate endoribonuclease L (RNAse L), which degrades all cellular RNA—including both viral and cellular. This results in apoptosis, which is evident in the present case. This effect is much bigger and much more significant than just an anti-viral or replication inhibition effect that allows for cell survival.

OAS1 transcript levels were significantly enhanced by treatment with CBD in cells expressing control plasmid, or ORF8, ORF10, or M-protein, versus vehicle control treatment. This would be expected to significantly enhance apoptosis in response to viral presence, or to prime cells to be prepared for a viral infection, allowing a more rapid anti-viral, pro-apoptotic response to viral infection.

Augmenting induction of OAS1 gene is associated with a dramatic protection against SARS-CoV-2 (and people with higher expression are less likely to get sick).

Thus, to conclude, Cannabidiol has multiple pathways through which it enhances immune response of the host cells. It prepares host cell for viral threat and can act as a prophylactic medicine. The minor increase in OAS1 expression and INF-gamma in control-transfected cells treated with CBD indicates a potential that CBD ‘primes’ cells to be ready to respond to a viral threat, without actually increasing apoptosis. On the other hand, remarkable increase in interferons and interferon-induced effector genes have been found to enhance immune response of the cell when cells transfected with viral proteins are treated with Cannabidiol. Cannabidiol causes early and late apoptosis in cells transfected with ORF8 and M protein.

Whether apoptosis is due to Cannabidiol alone or through increased levels of Type III interferons (i.e. Lambda-type interferons), which tend to force cells towards apoptosis, it makes infected host cells not available to the virus to replicate and mutate.

Cannabidiol can also improve outcomes of existing immunization strategies for COVID-19 including but not restricted to by reducing the chances of transmission of viral particles following vaccination and before a full immune response in the individual is mounted, while also preventing the expansion of the viral gene pool through prevention of mutation.

Indeed, even after immunity has been acquired from vaccination, individuals who contract SARS-CoV-2 can still generate novel variants when mutation occurs during viral replication, since viral replication occurs during the time between cell infection, and activation of an effective and full humoral acquired (also called adaptive) immune response. This activation can take hours to days, and therefore even vaccinated people can still spread the virus, and produce mutants, during this interval. By augmenting apoptosis in cells exposed to viral genes, Cannabidiol can prevent viral replication and therefore the formation of novel SARS-CoV-2 variants.

Cannabidiol can also be candidate for including but not restricted to prophylaxis for travelers, essential workers and other high risk individuals to potentially control the spread of the virus within the host as well as transmission to others. Further the potential to prevent mutations becomes significant, especially for travelers who may be susceptible to introducing non indigenous strains into new geographies which may increase variants.

Cannabidiol also has regulatory approval for pediatric use in patients as young as 1 year of age for rare forms of epilepsy. Therefore, its potential for use in children who may be asymptomatic carriers and/or reservoirs of Sars-CoV-2 and other viruses, cannot be undermined, for prophylaxis, to reduce chances of community spread and increased variants and mutations. Cannabidiol can potentially be also administered to new born babies as a mono-treatment with Cannabidiol or as a prophylactic or as an adjunct/concomitant therapy for Sars-CoV-2 and other viruses.

In an embodiment, invention provides a pharmaceutical composition comprising therapeutically effective amount of Cannabinoid for use in treatment of Covid-19 infectious disease caused by Sars-Cov-2 virus wherein administration of said pharmaceutical composition to the said patient suffering from Covid-19 produces an enhancement/augmentation of innate immunity of the patient due to at least one of the following effects,
i) infected patient cells undergo apoptosis early after infection;
ii) induction of interferon transcription in the patient;
iii) induction of interferon-induced antiviral effectors in the patient.

In another embodiment, the invention provides a pharmaceutical composition comprising therapeutically effective/prophylactically effective amount of Cannabinoid for use in prophylaxis or prophylactic treatment of Covid-19 wherein administration of said pharmaceutical composition to an animal/human produces an enhancement/augmentation of innate immunity in such animal/human due to at least one of the following effects,
i) infected animal or human cells undergo apoptosis early after infection;
ii) induction of interferon transcription in the animal/human;
iii) induction of interferon-induced antiviral effectors in the animal/human.

In one more embodiment, the invention provides A pharmaceutical composition for treatment or prophylaxis or prophylactic treatment of animal/human/patient for Covid-19 infectious disease caused by Sars-Cov-2 virus wherein administering the said pharmaceutical composition to said animal/human/patient produces an enhancement/augmentation of innate immunity of the animal/human/patient due to at least one of the following effects,
i) infected cells undergo apoptosis early after infection;
ii) induction of interferon transcription in the patient;
iii) induction of interferon induced antiviral effector in the patient;
and wherein one or more of the above effects causes at least one of the following,
i) clearing viruses partially or completely;
ii) preventing the development of infection, and raising the infectious titre needed to cause disease;
iii) preventing viral replication, and therefore mutant (variant) formation.

One more embodiment provides a pharmaceutical composition comprising therapeutically effective amount of one or more Cannabinoids for preventing or reducing mutation of Sars-Cov-2 virus in a patient by administration of said pharmaceutical composition to the said patient suffering from Covid-19 infectious disease wherein administration of said pharmaceutical composition to said patient produces an enhancement/augmentation of innate immunity in such animal/human due to at least one of the following effects,
i) infected animal or human cells undergo apoptosis early after infection;
ii) induction of interferon transcription in the animal/human;
iii) induction of interferon-induced antiviral effectors in the animal/human.

Yet another embodiment of the invention provides a pharmaceutical composition comprising therapeutically effective amount of one or more Cannabinoids for better preparing an animal or a human for Covid-19 infectious disease wherein said animal/human is about to get infected with Covid-19 infectious disease by administration of said pharmaceutical composition to the said animal/human wherein administering the said pharmaceutical composition to said animal/human produces an enhancement/augmentation of innate immunity of the animal/human due to at least one of the following effects,
i) infected cells undergo apoptosis early after infection;
ii) induction of interferon transcription in the patient;
iii) induction of interferon induced antiviral effector in the patient.

One other embodiment of the present invention covers a method of treating Covid-19 infectious disease caused by Sars-Cov-2 virus in a patient wherein the said method comprises administering to said patient, a pharmaceutical composition comprising therapeutically effective amount of Cannabinoid wherein the administration of said pharmaceutical composition to the said patient produces an enhancement/augmentation of innate immunity of the patient due to at least one of the following effects,
i) infected patient cells undergo apoptosis early after infection;
ii) induction of interferon transcription in the patient;
iii) induction of interferon induced antiviral effector in the patient.

One more method according to the present invention is a method of prophylaxis or prophylactic treatment of Covid-19 infectious disease caused by Sars-Cov-2 virus wherein said method comprises administering a pharmaceutical composition comprising therapeutically effective/prophylactically effective amount of a Cannabinoid to an animal/human wherein administration of said pharmaceutical composition produces an enhancement/augmentation of innate immunity in such animal/human due to at least one of the following effects,
i) infected animal or human cells undergo apoptosis early after infection;
ii) induction of interferon transcription in the animal/human;
iii) induction of interferon-induced antiviral effectors in the animal/human.

Yet another embodiment provides a method of treating or a method of prophylaxis or prophylactic treatment for Covid-19 infectious disease caused by Sars-Cov-2 virus wherein the said method comprises administering to said animal/human/patient, a pharmaceutical composition comprising prophylactically/therapeutically effective amount of Cannabinoid wherein the administration of said pharmaceutical composition to the said animal/human/patient produces an enhancement/augmentation of innate immunity of the animal/human/patient due to at least one of the following effects,
i) infected cells undergo apoptosis early after infection;
ii) induction of interferon transcription in the patient;
iii) induction of interferon induced antiviral effector in the patient; and wherein one or more of the above effects causes at least one of the following,
i) clearing viruses partially or completely;
ii) preventing the development of infection, and raising the infectious titre needed to cause disease;
iii) preventing viral replication, and therefore mutant (variant) formation.

Results

Results of 2 μM Cannabidiol

1. Relative Cell Numbers

A dose response curve was generated by treating cells transfected with the control plasmid (pCMV) or plasmids expressing viral proteins with vehicle (0.1% EtOH) or increasing concentrations of CBD. The range of concentrations tested was based on pharmacologically achievable blood concentrations observed in human pharmacokinetic studies [50]. A dose-dependent decrease in relative cell number was observed in cells expressing SARS-CoV-2 proteins, but not in cells transfected only with the control plasmid (FIG. 8D). Overall effects on relative cell number at each concentration were highly similar among the different viral proteins tested. Specific analyses are shown comparing cells transfected with the control vector or plasmids expressing viral proteins, and treated with either vehicle or 2 μM CBD, which showed the greatest effect (FIG. 8E, 8E, 8G). Treatment of cells with 2 μM CBD did not significantly affect the relative number of control plasmid-transfected cells at 24 h compared to treatment with vehicle alone. In cells expressing ORF8, ORF10, or M protein and treated with vehicle alone, the relative number of cells per well was not significantly different from the number of cells in wells transfected with control plasmid. However, in cells expressing ORF8, ORF10, or M protein and treated with 2 μM CBD, there was a significant reduction in the relative cell number per well.

2. Early and Late Apoptosis

Differences in cell number can result from changes in cell proliferation, or cell death (i.e. apoptosis or necrosis), or both. The inventors initially assayed both cell proliferation and apoptosis using cells treated with either vehicle or 1 μM CBD, and found a >60% increase in apoptosis indexes, but no significant effect on cell proliferation (data not shown). Inventors therefore focused our studies on apoptosis. A dose-dependent effect of CBD on the activation of an early marker of apoptosis (pSIVA), and incorporation of a late marker of apoptosis (PI), was evident in cells expressing ORF8, ORF10, and M protein, but this was not observed in cells transfected only with the control plasmid (FIGS. 9G, 9H). Specific analyses comparing cells transfected with the control vector or plasmids expressing viral proteins, and treated with either vehicle or 2 μM CBD (FIG. 9I-9N), demonstrate several important effects. First, this analysis shows that CBD alone, even at the highest concentration tested, does not significantly increase apoptosis in control cells. Additionally, it demonstrates that expression alone of the viral proteins ORF8, ORF10, or M protein, also does not significantly increase either early or late apoptosis relative to control cells, indicating a poor ability of cells to detect and respond to the presence of these viral RNAs or proteins. Interestingly, however, in cells expressing ORF8, early and late apoptosis indexes were both increased by over 6-fold in cells treated with 2 μM CBD compared to indexes in vehicle alone (FIG. 9I, 9L). In cells expressing ORF10 (FIG. 9J, 9M), early and late apoptosis were increased ˜4.7- and ˜4.0-fold, respectively, while these respective increases were ˜5.6- and ˜4.7-fold in cells expressing M protein (FIG. 9K, 9N).

3. Expression of INF Genes

Expression of the Type I INFs (INFα and INFβ) was not significantly altered by ORF8, ORF10, or M protein, either with or without 2 μM CBD (FIG. 10F-10K). However, the presence of viral proteins significantly increased gene expression of Type II INF (INF γ and Type III INFs (INFλ1 and INFλ2/3), and this was further augmented by 2 μM CBD (FIG. 11C-11K). Although relative cell number and apoptosis measures were not significantly affected by 2 μM CBD, this treatment caused an ˜5-fold increase in expression of INFγ in pCMV-control cells compared to pCMV-controls cells treated only with vehicle (FIG. 11C-11E). Similarly, INFλ1 and INFλ2/3 were increased in pCMV-transfected control cells treated with 2 μM CBD by 3-fold (FIG. 11F-11H) and 7-fold (FIG. 11I-11K), respectively. In the absence of CBD, transfection of cells with ORF8, ORF10, or M protein caused a significant 16- to 29-fold increase in expression of INF γ relative to vehicle-treated control cells, but this effect was augmented by treatment with 2 μM CBD, further increasing INF γ expression by another 1.5 to 3.3-fold (FIG. 11F-11H). Interestingly, however, cells transfected with ORF8 (in the absence of CBD) did not have higher expression of INF λ1 or INF λ2/3, although these genes were induced without CBD co-treatment when cells were transfected with ORF10 (by 9.6-fold and 2.4-fold) or M protein (by 4.1-fold, for both genes). Treatment of cells with 2 μM CBD further augmented the induction of INFλ1, but not INFλ2/3, by ORF8. Conversely, 2 μM CBD strongly augmented the induction of both INFλ1 and INFλ2/3 that occurred when ORF10 or M protein were transfected, by a further 3.8- to 11.2-fold.

4. Expression of ISG

Expression of the ISGs IFIT and Mx was not significantly altered by treatment with 2 μM CBD, or by expression of the SARS-CoV-2 proteins ORF8, ORF10, and M protein, either alone, or in combination (FIG. 12B-12G). However, several interesting effects were observed when OAS family genes were analyzed. Surprisingly, expression of ORF8, ORF10, and M protein did not significantly induce expression of OAS1, OAS2, or OAS3 relative to cells transfected with pCMV in the absence of CBD (FIG. 15A-15I). This indicates that these cells may have a poor ability to recognize and respond to these viral proteins through innate immune system activation involving the OAS family. Only OASL was significantly induced by ORF8 (by 7.8-fold), ORF10 (by 4.87-fold), and M protein (by 18.84-fold), in the absence of CBD (FIG. 16A-16C). When 2 μM CBD was added to cells transfected with the control plasmid, expression of OAS2, OAS3, and OASL increased significantly (from 5.7 to 7.8-fold). Addition of 2 μM CBD to cells transfected with ORF8, ORF10, or M protein, augmented the expression of all OAS family genes relative to the corresponding vehicle-treated cells, with the additional induction caused by CBD ranging from 3.1- to 22.9-fold.

Pharmaceutical Compositions:

A suitable dose/prophylactically/therapeutically effective amount of Cannabidiol (CBD) is from 0.00001 mg/kg of body weight to 4000 mg/kg of body weight.

The suitable dose/prophylactically/therapeutically effective amount of Cannabidiol can also be 0.00001 to 1000 mg/kg of body weight or 0.00001 to 500 mg/kg of body weight. The preferred dose/preferred prophylactically/therapeutically effective amount of Cannabidiol can be 0.00001 to 100 mg/kg of body weight or from 0.00001 to 10 mg/kg of body weight.

The dose will depend on the nature and status of human or animal patient health. It will also depend on age and comorbidities if any. Further, dose will depend on type of composition for example, whether oral or parenteral or topical.

Following pharmaceutical formulations/compositions are described for better understanding of the invention and they do not limit scope of the invention in any way.

Suitable oral dosage forms include but are not restricted to tablets—sublingual, buccal, effervescent, chewable; troches, lozenges, dispersible powders or granules and dragees; capsules, solutions, suspensions, syrups, lozenges, medicated gums, buccal gels or patches. Tablets can be made using compression or molding techniques well known in the art. The other dosage forms can also be prepared by 3Dimensional (3D) or 4D printing and also by Carbon graphene loaded nano-particles and micro-particles. Gelatin or non-gelatin capsules can be formulated as hard or soft capsule shells, which can encapsulate liquid, solid, and semisolid fill materials, using techniques well known in the art.

Following examples provide various Pharmaceutical compositions of the Cannabidiol (CBD)

The Oral Spray formulation encompasses the Cannabidiol (CBD); each at concentration of 0.00001 mg to 200 mg/ml and have excipients such as diluents viz. Mannitol ranging from 10-15 mg/ml; Sweeteners such as sucralose from 5-10 mg/ml, Flavours as Raspberry, Strawberry from 5-10 mg/ml and tonicity and taste modulators such as sodium chloride and propylene glycol from 0.1-0.5 mg/ml with purified water as the base solvent or carrier. The specific gravity of the formulation can be between 1.01 to 1.5 g/ml

Additionally, the said Oral Spray may encompass surfactant-solubilizers and gelling agents such as Pluronic F127 or Poloxamer 407 in the concentration ranging from 1-200 mg/ml. This formulation is liquid at temperatures less than 10 degree Celsius and starts gelling at temperature range above 30 degree Celsius. It is a sterile, nonpyrogenic solution. The pH range if reconstituted should be 5-9 preferably 6.5-7.5. It can be administered using appropriate spray containers with specialised spray nozzle to facilitate spray below the tongue viz. sublingually or into the buccal or also the nasal cavity. The Spray can also be in the form of a micronized or nanosized suspension. The Nasal Spray formulation would be devoid of the sweeteners and flavours. It gels at body temperature thereby facilitating longer dwell time possibly enhancing the drug penetration through the mucosal lining. This drug delivery mode bypasses the harsh acidic conditions of the stomach and also the hepatic breakdown thereby possibly increasing bio-availability. The specific gravity of the formulation can be between 1.01 to 1.7 g/ml

The Injection formulation contains the Cannabidiol (CBD); at concentration of 0.00001 mg to 200 mg/ml and solubilizers such as Ethyl alcohol 20%/ml and Propylene glycol 40%/ml and Water for injection ˜40%/ml. The solution should be isotonic and tonicity adjusting salts such as sodium chloride can be used. The pH range of 5-9 can be adjusted with suitable bufferants should be 6-8 preferably 6.5-7.5. It is a sterile, nonpyrogenic solution. The said Injection formulation can be in the form of a solution or micronized or nanosized dispersion. The said formulation can also be administered via inhalation with or without the aid of a medical device, metered or unmetered, and/or via nebulization for nasal administration for drug delivery to the lungs—viz. Pulmonary. The said formulation can also be administered via the buccal route as buccal drops or as buccal spray using appropriate medical device. The said formulation can be administered via the sublingual route as sublingual drops or as sublingual spray using appropriate medical device. Another variant of the sterile injectable formulation can also be a lyophilized injection. This injection may also contain sodium citrate dihydrate and citric acid anhydrous; and finally, be as a white to yellow lyophilized powder or plug. The solution should be prepared only with 1 to 2 mL of preservative-free Sterile Sodium Chloride Injection, 0.9 percent or preservative-free Sterile Water for Injection. The reconstituted solution is clear, slightly yellow and essentially free from visible particles. The specific gravity of the formulation can be between 1.01 to 1.7 g/ml. The particle size of the liquid droplets can range from 5 micron to 500 micron.

The Inhalation or Pulmonary Capsule has the Cannabidiol (CBD) concentration of 0.00001 mg to 50 mg/capsule and has excipients such as Magnesium stearate [Inhalation grade] or Lactose [Inhalation grade]. The core weight of the formulation can range from 25-500 mg/capsule.

The Aerosol or Pulmonary delivery system has the Cannabidiol (CBD) concentration of 0.00001 mg to 100 mg/actuation and has excipients such as propellent gases, propylene glycol, water, surfactants, anti-foam emulsion and anti-freeze excipients. The particle size of the liquid droplets can range from 5 micron to 500 micron.

Sublingual Tablets have the Cannabidiol (CBD); at concentration of 0.00001 mg to 50 mg/tablet and have excipients such as diluents viz. Lactose monohydrate or Mannitol ranging from 10-30 mg/tablet;

Disintegrants such as Starch or Crospovidone from 10-15 mg/tablet; fillers such as Microcrystalline cellulose from 5-10 mg/tablet and lubricants such as Magnesium stearate from 0.5-1 mg/tablet. It may additionally contain or 5-10 mg/tablet of taste modulating or masking agents such as sodium chloride or buffers such as potassium dihydrogen phosphate. The core weight of the formulation can range from 50-80 mg/tablet.

The Orally Dispersible Tablets (ODT) have the Cannabidiol (CBD) at concentration of 0.00001 mg to 100 mg/tablet and have excipients such as diluents viz. Lactose monohydrate or Mannitol ranging from 10-15 mg/tablet; Disintegrants such as Starch or Crospovidone from 10-15 mg/tablet; fillers such as Microcrystalline cellulose from 5-10 mg/tablet and lubricants such as Magnesium stearate from 0.5-1 mg/tablet. The core weight of the formulation can range from 50-80 mg/tablet.

The Buccal Tablets have the Cannabidiol (CBD) at concentration of 0.00001 mg to 100 mg/tablet and have excipients such as polymers viz. polymers of acrylic acid and C10-C30 alkyl acrylate crosslinked with allyl pentaerythritol exemplified by Carbopol 934 ranging from 10-15 mg/tablet and or Hydroxy Propyl Methyl Cellulose (HPMC) K4M from 35-40 mg/tablet; Fillers such as Mannitol (directly compressible) from 10-15 mg/tablet; and lubricants such as Magnesium stearate from 0.5-1 mg/tablet. The core weight of the formulation can range from 50-80 mg/tablet.

The Delayed Release Tablets have the Cannabidiol (CBD) at concentration of 0.00001 mg to 200 mg/tablet and have excipients such as Mannitol, Microcrystalline cellulose (MCC PH 102), trisodium phosphate, Hydroxy Propyl Methyl Cellulose (HPMC 5 cps), Hydroxy Propyl Methyl Cellulose (HPMC 15 cps) and Crospovidone, Colloidal silicon dioxide, Magnesium stearate as the tablet core coated with a Seal Coating composition encompassing Ethyl cellulose using an appropriate solvent system viz. aqueous, non-aqueous; preferably non-aqueous (Iso-propyl alcohol and Dichloromethane) to a 4-5% weight gain on the tablet cores finally coated with an aqueous gastro-resistant coating composition viz. Eudragit L100-55, Triethyl citrate, opacifier and colorant to a total weight gain of 26-30% of the tablet cores. The core weight of the formulation can range from 50-1200 mg/tablet.

The Extended Release Tablets have the Cannabidiol (CBD) at concentration of 0.00001 mg to 200 mg/tablet and have excipients such as fillers viz. Microcrystalline cellulose (MCC PH 101); polymers viz Hydroxy Propyl Methyl Cellulose (HPMC K100M) and Hydroxy Propyl Methyl Cellulose (HPMC K15M); binders viz. Povidone (PVP K29/32) and Lubricants viz. Magnesium stearate as the tablet core coated with a Film Coating composition using an appropriate solvent system viz. aqueous or non-aqueous; preferably non-aqueous (Iso-propyl alcohol and Dichloromethane) to a 2-3% weight gain on the tablet core. The core weight of the formulation can range from 50-1200 mg/tablet.

The Effervescent tablets have the Cannabidiol (CBD) at concentration of 0.00001 mg to 200 mg/tablet and have excipients such as citric acid, sodium bicarbonate, potassium citrate, mannitol, aspartame, strawberry flavour, bufferants, sodium benzoate and polyethylene glycol 6000. The core weight of the formulation can range from 50-2000 mg/tablet.

The Osmotic-controlled Release Oral delivery System (OROS) Tablets have the Cannabidiol (CBD) at concentration of 0.00001 mg to 200 mg/tablet and have excipients such as sorbitan monolaurate and Sodium chloride, microcrystalline cellulose (MCC PH 102), polymers viz Hydroxy Propyl Methyl Cellulose (HPMC K100M) and Hydroxy Propyl Methyl Cellulose (HPMC K15M), Colloidal silicon dioxide and Magnesium stearate as the tablet core; a Film coat to the tablet cores to a weight gain of 2.5 to 3.0% w/w to the tablet core using a non-aqueous medium and a Functional Coat the tablet with Cellulose acetate non-aqueous dispersion in Iso-propyl alcohol to a weight gain of 25-30% w/w of the tablet core finally Laser drilled the tablets with an orifice of 150-250 micron. The core weight of the formulation can range from 50-1000 mg/tablet.

The Capsules have the Cannabidiol (CBD) at concentration of 0.00001 mg to 200 mg/capsule and have excipients such a microcrystalline cellulose (MCC PH 105), Colloidal silicon dioxide and Magnesium stearate as the core; encompassed in a hard gelatin capsule. The core weight of the formulation can range from 30-2055 mg/capsule.

The Compressed lozenges or Chews or Lollipop have the Cannabidiol (CBD) at concentration of 0.00001 mg to 200 mg/unit and have excipients such as ethoxylated hydrogenated castor oil Polyoxyl 35 Castor oil (Cremophore EL/Kolliphor EL), Dextrate, Polyethylene glycol 6000, microcrystalline cellulose (MCC 102), povidone (PVP K29/32) and FD&C Yellow No. 6 and Magnesium stearate as the core. The core weight of the formulation can range from 100-3000 mg/unit

The Soft Gel Capsules have the Cannabidiol (CBD) at concentration of 0.00001 mg to 200 mg/capsule and have excipients such as propylene glycol, Poly Ethylene Glycol-400, Polyvinyl pyrrolidone K29/32, Butylated hydroxy toluene and ethanol-water blend as the core material filled into opaque soft gelatin capsules. The core weight of the formulation can range from 100-800 mg/capsule.

The Quick dissolving film—Oral and or Sublingual, have the Cannabidiol (CBD) at concentration of 0.00001 mg to 200 mg/unit and have excipients such as pullulan, sorbitol, polysorbate 80, sucralose, Monoammonium glycyrrhizinate and peppermint flavour. The core weight of the formulation can range from 50-800 mg/unit

The Oro-Buccal muco-adhesive film—Oral or Sublingual, have the Cannabidiol (CBD) at concentration of 0.00001 mg to 200 mg/unit and have excipients such as Hydroxypropyl cellulose, Hydroxyethyl cellulose and Sodium carboxymethyl cellulose, Polyoxyl 35 Castor Oil (Cremophore EL/Kolliphor EL), Sodium benzoate, Parahydroxybenzoate methyl, Parahydroxybenzoate propyl, Sodium citrate and Sodium saccharine. The core weight of the formulation can range from 50-80 mg/unit.

The Oral Emulsion has the Cannabidiol (CBD) at concentration of 0.00001 mg to 200 mg/g and have excipients such as Polyoxyl 35 Castor Oil (Cremophore EL/Kolliphor EL), Saccharin Sodium, caramel, colorant, peppermint oil, corn oil, sucrose and water. The specific gravity of the formulation can be between 0.5-1.5 g/ml

The Vaginal gel has the Cannabidiol (CBD) at concentration of 0.00001 mg to 200 mg/g and has excipients such as Polyoxyl 35 Castor Oil (Cremophore EL/Kolliphor EL), ascorbic acid, Glycerin or Propylene glycol, Hydroxypropyl Methylcellulose (HPMC E50), Trisodium Citrate dihydrate and water. The specific gravity of the formulation can be between 1.01-1.8 g/ml.

The Eye drop formulation has the Cannabidiol (CBD) at concentration of 0.00001 mg to 200 mg/ml and has excipients such as Polysorbate 20/80, Benzalkonium chloride, disodium EDTATE, Sodium Carboxymethyl Cellulose (Na CMC), Citric acid monohydrate, sodium hydroxide, hydrochloric acid and water. The final solution is sterile. The specific gravity of the formulation can be between 1.01-1.8 g/ml.

The Suppository formulation has the Cannabidiol (CBD) at concentration of 0.00001 mg to 200 mg/g and has excipients such as hard fat, surfactants, and the following inactive ingredients: butylated hydroxy anisole, butylated hydroxytoluene, edetic acid, glycerin, polyethylene glycol 3350, polyethylene glycol 8000, purified water and sodium chloride. The core weight of the formulation can range from 200-3000 mg/unit

### EXAMPLES

### Example 1: Process for Measuring Cell Proliferation Rates

The bromodeoxyuridine incorporation rate was measured by incorporating and quantifying bromodeoxyuridine (BrdU) into DNA of actively proliferating cells. The absorbance values are measured by ELISA assay with a BioTek Synergy H1 Hybrid Multi-Mode Microplate reader assay at 370 nm (reference wavelength: approx. 492 nm). FIGS. 1-5 provide results of the tests performed. Note, however, that cell proliferation level can only be inferred once the data are normalized to cell number. FIG. 6 combines data from all figures for ready comparison. The absorbance is expressed as % untreated control. In FIG. 7, data are normalized to relative cell number.

### Example 2—Crystal Violet Staining

Crystal Violet Staining

Relative cell numbers were quantified using the crystal violet staining method, as previously described by Duncan, R. E., et al [Duncan, R. E., et al, 2004]. Briefly, HEK293 cells were seeded (1×10⁴ cells) in 96-well plates and transfected with the respective plasmids after 24 h, then treated a few hours after transfection with either CBD or vehicle for 24 h. Cells were gently washed with 1× phosphate buffered saline (PBS), fixed with a mixture of 10% methanol 10% acetic acid (v/v) and stained with crystal violet (Fisher Scientific; #AAB2193214), then washed and eluted for measurement of absorbance of the samples using a BioTek Synergy H1 Hybrid Multi-Mode Microplate reader at 595 nm.

### Example 3: Apoptosis Assay

Early and late apoptotic cells were detected using a Kinetic Apoptosis Kit (#ab129817, Abcam, Toronto, Ontario, Canada), according to the manufacturer's instructions. Briefly, cells were seeded (1×104 cells) in 96-well plates, transfected after 24 h, and treated with either CBD or vehicle for 24 hours, then labelled with Polarity Sensitive Indicator of Viability & Apoptosis (pSIVA™), which detects early/ongoing apoptosis, and with Propidium Iodide (PI), which detects cells that are in late apoptosis. Live cells were maintained at 37° C. while fluorescence was recorded at 469/525 nm for the detection of pSIVA and at 531/647 nm for the detection of PI. Results are expressed as an index, with the early apoptosis index calculated as pSIVA absorbance at 525 nm/relative cell number per well, and the late apoptosis index calculated as PI absorbance at 647 nm/relative cell number per well.

### Example 4: Methods for Measuring Levels of Interferons and Effectors Gene Expression

INF and ISG mRNA Expression

qPCR analysis was conducted as as one of the inventors have previously described (M'Hiri I et al., 2020). Cells were grown in 24 well plates and transfected with either pCMV-3Tag-3A, or plasmids expressing ORF8, ORF10, or M protein, and then treated with either 2 [symbol] M CBD or vehicle overnight for 14 h. Total RNA was isolated using TRIzol® Reagent (1 ml per well) as described by the manufacturer (Invitrogen, Waltham, MA). Quantification of RNA samples was performed using a Nanodrop 2000 Spectrophotometer (Thermo Fisher, Waltham, MA), and 2 μg of RNA was used to synthase cDNA via oligo(dT) priming using SuperScript II Reverse Transcriptase, according to the manufacturer's protocol (Invitrogen, Waltham, MA). For the real-time PCR assays, cDNA was diluted 1:4 and 1 μl was added to a master mix with 9 μl of PerfeCTa SYBR© Green supermix (Quanta Bio, Beverly, MA), 0.5 μl forward and reverse primers (25 μM each) of the targeted gene (please see list below), and 3 μl of ddH₂0. The cycling conditions for all genes were as follows: 1 cycle of 95° C. for 2 min, followed by 49 cycles of 95° C. for 10 s, then 60° C. for 20 s. Relative expression of the targeted gene was calculated using the Ct method with the Ct values normalized to glyceraldehyde 3-phosphate dehydrogenase (Gapdh).

TABLE 6
Primer sequences

| Gene primer | Sequence (5′-3′) |
|---|---|
| IFN-gamma-Forward | TGGCTTTTCAGCTCTGCATC |
| IFN-gamma-Reverse | CCGCTACATCTGAATGACCTG |
| IFN-lambda 1-Forward | GAGGCCCCCAAAAAGGAGTC |
| IFN-lambda 1-Reverse | AGGTTCCCATCGGCCACATA |
| IFN lambda 2-3-Forward | CTGCCACATAGCCCAGTTCA |
| IFN lambda 2-3-Reverse | AGAAGCGACTCTTCTAAGGCATCTT |
| Mx-Forward | GGCTGTTTACCAGACTCCGACA |
| Mx-Reverse | CACAAAGCCTGGCAGCTCTCTA |
| IFIT-Forward | GGAATACACAACCTACTAGCC |
| IFIT-Reverse | CCAGGTCACCAGACTCCTCA |
| OAS1-Forward | GAAGGCAGCTCACGAAACC |
| OAS1-Reverse | AGGCCTCAGCCTCTTGTG |
| OAS2-Forward | TTCTGCCTGCACCACTCTTCACGA |
| OAS2-Reverse | GCCAGTCTTCAGAGCTGTGCCTTTG |
| OAS3-Forward | CCGAACTGTCCTGGGCCTGATCC |
| OAS3-Reverse | CCCATTCCCCAGGTCCCATGTGG |
| OASL-Forward | GACGAAGGCTTCACCACTGT |
| OASL-Reverse | GTCAAGTGGATGTCTCGTGC |
| Gapdh-Forward | AGAAGGCTGGGGCTCATTTG |
| Gapdh-Reverse | AGGGGCCATCCACAGTCTTC |

Example 5-CANNABIDIOL FILM COATED TABLETS

| A | TABLET CORE | |
|---|---|---|
| 1 | Cannabidiol | 0.1 mg to 100 mg or |
| | | 100 mg to 200 mg |
| 2 | Microcrystalline cellulose (MCC PH105) | 40% of the total core |
| | | weight |
| 3 | CELLULOSE METHYLHYDROXYPROPYL | 2% of the total core |
| | 5CPS | weight |
| 4 | COLLOIDAL SILICON DIOXIDE | 2% of the total core |
| | | weight |
| 5 | POLYVINYL PYROLLIDONE (PVP K29/32) | 2% of the total core |
| | | weight |
| 6 | MAGNESIUM STEARATE | 0.5% of the total core |
| | | weight |
| B | FILM-COATING | |
| | Consists of Polyvinyl alcohol, polyethylene glycol, | 2.0-2.5% of the total core |
| | Talc, Opacifier, lecithin reconstituted to 10% w/w | weight |
| | dispersion in water-Iso-propyl alcohol blend *or Iso- | |
| | propyl alcohol* | |
| | * = Evaporates during tablet coating and is not | |
| | present substantially in the final product-The film | |
| | coated tablet. | |
| C | PROCESS: | |

Co-sift Cannabidiol and MCC PH 105, Cellulose methyl hydroxypropyl and
polyvinyl-pyrollidone through ASTM # 40 mesh twice. Label it as Mix A.
Sift individually the colloidal silicon dioxide and the magnesium stearate through
ASTM # 40 and collect in separate polybags.
Transfer the Mix A to a V-blender of appropriate size allowing 60% of its
occupancy. Blend at 15 RPM for 10 minutes. Label it as Mix B.
Add the pre-sifted colloidal silicon dioxide the Mix B in the blender and continue
to blend at 10 RPM for 5 minutes. Label it as Mix C.
Add the pre-sifted magnesium stearate to the Mix C in the blender and continue
to blend at 10 RPM for 2 minutes.
Unload the final blend into a double LDPE polybag lined with a black polybag
on the outermost side. Displace the air inside each bag and tie each one with a
nylon tag. Keep 5 dessicant pillow pouches (each with 100 gm capacity) in the
second outer bag before tying it up with a nylon tie. Finally, put the double
polybag with the Mix into a black polybag and secure with a nylon tie. Label the
final bag as ″Lubricated Blend ready for compression″. Use appropriate
compression tooling to compress the Lubricated Blend into biconvex tablets of
appropriate hardness so that the percent friability is less than 0.5% w/w and the
disintegration time (DT) is not more than 15 minutes.
Further coat the tablets in an appropriate tablet coater/coating machine with the
hydro-alcoholic or alcoholic tablet coating dispersion of appropriate sprayable
consistency to achieve a weight gain of 2.0-2.5% w/w on the tablet core.
Fill the film-coated tablets into appropriate well-filled, opaque white/coloured
containers (of appropriate material) so that there is minimum head space along
with appropriate protectants against moisture and oxygen.

Example 6: CANNABIDIOL CAPSULES

| A | CORE INGREDIENTS | |
|---|---|---|
| 1 | Cannabidiol | 0.1 mg to 100 mg or 100 mg to |
| | | 200 mg |
| 2 | Microcrystalline cellulose (MCC PH105) | 40% of the total capsule core |
| | | weight |
| 3 | CELLULOSE | 2% of the total capsule core |
| | METHYLHYDROXYPROPYL 5CPS | weight |
| 4 | COLLOIDAL SILICON DIOXIDE | 2% of the total capsule core |
| | | weight |
| 5 | POLYVINYL PYROLLIDONE (PVP | 2% of the total capsule core |
| | K29/32) | weight |
| 6 | MAGNESIUM STEARATE | 0.5% of the total capsule core |
| | | weight |
| B | ENCAPSULATION | |
| | Consisting of opaque, coloured, Hydroxy- | |
| | propyl methyl cellulose (HPMC) of | |
| | appropriate size viz. 00el to 5 to encompass | |
| | or encapsulate the ingredients. | |

| C | PROCESS: |
|---|---|
| | Co-sift Cannabidiol and MCC PH 105, cellulose methyl hydroxypropyl and |
| | polyvinyl-pyrollidone through ASTM # 40 mesh twice. Label it as Mix A. |
| | Sift individually the colloidal silicon dioxide and the magnesium stearate |
| | through ASTM # 40 and collect in separate polybags. |
| | Transfer the Mix A to a V-blender of appropriate size allowing 60% of its |
| | occupancy. Blend at 15 RPM for 10 minutes. Label it as Mix B. |
| | Add the pre-sifted colloidal silicon dioxide the Mix B in the blender and |
| | continue to blend at 10 RPM for 5 minutes. Label it as Mix C. |
| | Add the pre-sifted magnesium stearate to the Mix C in the blender and continue |
| | to blend at 10 RPM for 2 minutes. |
| | Unload the final blend into a double LDPE polybag lined with a black polybag |
| | on the outermost side. Displace the air inside each bag and tie each one with a |
| | nylon tag. Keep 5 dessicant pillow pouches (each with 100 gm capacity) in the |
| | second outer bag before tying it up with a nylon tie. Finally, put the double |
| | polybag with the Mix into a black polybag and secure with a nylon tie. Label |
| | the final bag as ″Lubricated Blend ready for Capsule filling″. Use appropriate |
| | tooling and the Capsule filling machine to fill the Lubricated Blend into |
| | capsules of appropriate size such that the disintegration time (DT) is not more |
| | than 10 minutes. Label them as Finished Capsules. |
| | Fill the finished capsules into appropriate well-filled, opaque white/coloured |
| | containers (of appropriate material) so that there is minimum head space along |
| | with appropriate protectants against moisture and oxygen. |

Example 7
CANNABIDIOL INJECTION or CANNABIDIOL nasal drops or CANNABIDIOL
nasal spray or CANNABIDIOL buccal drops or CANNABIDIOL buccal
spray or CANNABIDIOL sublingual drops or CANNABIDIOL sublingual spray

| | | |
|---|---|---|
| 1 | Cannabidiol | 0.5-100 mg/ml |
| 2 | Propylene glycol | 30% |
| 3 | Ethyl alcohol | 20% |
| 4 | Sodium benzoate/benzoic acid | 5% |
| 5 | Benzyl alcohol | 1.50% |
| 6 | Water for injection | ~43% |
| | It is a sterile, nonpyrogenic solution. The pH range if | |
| | reconstituted should be 5-9 preferably 6.5-7.5 | |

Dissolve the Cannabidiol in ethanol under continuous stirring in a closed vessel.
Label it as Mix A.
Add the sodium benzoate/benzoic acid and benzyl alcohol to propylene glycol
under continuous stirring in a larger vessel. Slowly add water to it under stirring.
Label it as Mix B.
Add the Mix B to mix A under continuous stirring.
Continue stirring till a clear solution is formed. Filter the final clear solution
through a 0.2-micron filter to yield a sterile solution.
All activity is to be executed in a parenteral facility using aseptic process only.
Using aseptic filling fill and seal the sterile solution into ampoules of 1 ml
capacity under nitrogen purging and under subdued light or under a sodium
vapour lamp.
The said formulation can be administered via the nasal route as nasal drops or as
nasal spray using appropriate medical device.
The said formulation can be administered via inhalation with or without the aid
of a medical device, metered or unmetered, and/or via nebulization.
The said formulation can be administered via the buccal route as buccal drops or
as buccal spray using appropriate medical device.
The said formulation can be administered via the sublingual route as sublingual
drops or as sublingual spray using appropriate medical device.

Example 8
CANNABIDIOL INJECTION or CANNABIDIOL nasal drops or CANNABIDIOL
nasal spray or CANNABIDIOL buccal drops or CANNABIDIOL buccal
spray or CANNABIDIOL sublingual drops or CANNABIDIOL sublingual spray

| | | |
|---|---|---|
| 1 | Cannabidiol | 0.5-100 mg/ml (active) |
| 2 | Ethyl alcohol | 20% of the active |
| 3 | Propylene glycol | 40% of the active |
| 4 | Water for injection | ~40% |
| | It is a sterile, nonpyrogenic solution with pH | |
| | range 4.0-7.0. The pH range if reconstituted | |
| | should be 5-9 preferably 6.5-7.5 | |

Dissolve the Cannabidiol in ethanol under continuous stirring in a small vessel.
Label it as Mix A.
Add the propylene glycol to mix A under continuous stirring in a larger vessel.
Slowly add water to it under stirring.
Continue stirring till a clear solution is formed. Filter the final clear solution through
a 0.2-micron filter to yield a sterile solution.
All activity is to be executed in a parenteral facility using aseptic process only.
Using aseptic filling fill and seal the sterile solution into ampoules of 1 ml capacity
under nitrogen purging and under subdued light or under a sodium vapour lamp.
The said formulation can be administered via the nasal route as nasal drops or as
nasal spray using appropriate medical device.
The said formulation can be administered via inhalation with or without the aid of a
medical device, metered or unmetered, and/or via nebulization.
The said formulation can be administered via the buccal route as buccal drops or as
buccal spray using appropriate medical device.
The said formulation can be administered via the sublingual route as sublingual
drops or as sublingual spray using appropriate medical device.

Example 9
CANNABIDIOL ear drops

| | | |
|---|---|---|
| 1 | Cannabidiol | 0.1-100 mg/ml |
| 2 | Iso-propyl alcohol | 95% |
| 3 | Glycerin | 5% |

Dissolve the Cannabidiol in iso-propyl alcohol under continuous stirring in a
closed vessel. Add the glycerin under continuous stirring.
Continue stirring till a clear solution is formed. Filter the final clear solution
through a 0.2-micron filter to yield a sterile solution.
All activity is to be executed in a parenteral facility using aseptic process only.
Using aseptic filling fill and seal the sterile solution into dark amber colored glass
vials or appropriate opaque-to-light containers of suitable material of 10 ml
capacity under nitrogen purging and under subdued light or under the light of a
sodium vapour lamp.
The said formulation can be administered via the auricular or otic route as ear
drops or can also be alternatively be administered as an intra-auricular spray using
an appropriate medical device.

Example 10
CANNABIDIOL ear drops

| | | |
|---|---|---|
| 1 | Cannabidiol | 0.1-100 mg/ml |
| 2 | Propylene glycol | 95% |

Dissolve the Cannabidiol in propylene glycol under continuous stirring in a closed
vessel.
Continue stirring till a clear solution is formed. Filter the final clear solution through
a 0.2-micron filter to yield a sterile solution.
All activity is to be executed in a parenteral facility using aseptic process only.
Using aseptic filling fill and seal the sterile solution into dark amber colored glass
vials or appropriate opaque-to-light containers of suitable material of 10 ml capacity
under nitrogen purging and under subdued light or under the light of a sodium vapour
lamp.
The said formulation can be administered via the auricular or otic route as ear drops
or can also alternatively be administered as an intra-auricular spray using an
appropriate medical device.

Example 11
CANNABIDIOL ear drops

| | | |
|---|---|---|
| 1 | Cannabidiol | 0.1-100 mg/ml |
| 2 | Industrial Methylated Spirits | 75% |
| | 95% (IMS) | |
| 3 | Glycerin | 5% |
| 4 | Polysorbate 80 | 2.5% |
| 5 | Sodium Hydroxide (for pH- | Quantity sufficient |
| | adjustment) | |
| 6 | Hydrochloric Acid (for pH- | Quantity sufficient |
| | adjustment) | |
| 7 | Purified Water | Quantity sufficient for 100% |

Dissolve the Cannabidiol, glycerin and polysorbate 80 in the IMS under
continuous stirring in a closed vessel. Add 90% the purified water to the solution
under stirring. Adjust the pH of the solution with 1N sodium hydroxide solution
and 1N hydrochloric acid to
a pH range between 6-7 under stirring. Add the
remaining water to the solution under continuous stirring.
Continue stirring till a clear solution is formed. Filter the final clear solution
through a 0.2-micron filter to yield a sterile solution.
All activity is to be executed in a parenteral facility using aseptic process only.
Using aseptic filling fill and seal the sterile solution into dark amber colored glass
vials or appropriate opaque-to-light containers of suitable material of 10 ml
capacity under nitrogen purging and under subdued light or under the light of a
sodium vapour lamp.
The said formulation can be administered via the auricular or otic route as ear
drops stored in a dark, amber colored, opaque-to-light container or can also be
alternatively be administered as an intra-auricular spray using an appropriate
medical device.

Example 12
CANNABIDIOL ear drops

| | | |
|---|---|---|
| 1 | Cannabidiol | 0.1-100 mg/ml |
| 2 | Polyethylene glycol | 75% |
| 3 | Sodium Hydroxide (for pH-adjustment) | Quantity sufficient |
| 4 | Hydrochloric Acid (for pH-adjustment) | Quantity sufficient |
| 5 | Purified Water | Quantity sufficient for 100% |

Dissolve the Cannabidiol in the polyethylene glycol under continuous stirring in a
closed vessel. Add 90% the purified water to the solution under stirring. Adjust
the pH of the solution with 1N sodium hydroxide solution and 1N hydrochloric
acid to a pH range between 6-7 under stirring. Add the remaining water to the
solution under continuous stirring.
Continue stirring till a clear solution is formed. Filter the final clear solution
through a 0.2-micron filter to yield a sterile solution.
All activity is to be executed in a parenteral facility using aseptic process only.
Using aseptic filling fill and seal the sterile solution into dark amber colored glass
vials or appropriate opaque-to-light containers of suitable material of 10 ml
capacity under nitrogen purging and under subdued light or under the light of a
sodium vapour lamp.
The said formulation can be administered via the auricular or otic route as ear
drops or can also alternatively be administered as an intra-auricular spray using an
appropriate medical device.

### REFERENCES

Lu R, Zhao λ, Li J, et al. Genomic characterisation and epidemiology of 2019 novel coronavirus: implications for virus origins and receptor binding. Lancet (London, England). 2020 February; 395(10224):565-574. DOI: 10.1016/s0140-6736(20)30251-8.
Zhou, P. et al, A pneumonia outbreak associated with anew coronavirus of probable bat origin, Nature. 2020 March; 579(7798):270-273. doi: 10.1038/s41586-020-2012-7. Epub 2020 Feb. 3.
Xu J, Zhao S, Teng T, et al. Systematic Comparison of Two Animal-to-Human Transmitted Human Coronaviruses: SARS-CoV-2 and SARS-CoV. Viruses. 2020; 12(2):244. Published 2020 Feb. 22. doi:10.3390/v12020244
Shi C S, Qi H Y, Boularan C, Huang N N, Abu-Asab M, Shelhamer J H, et al. SARS-coronavirus open reading frame-9b suppresses innate immunity by targeting mitochondria and the MAVS/TRAF3/TRAF6 signalosome. Journal of immunology. 2014; 193(6):3080-9.
Jin-Yan Li et al-Jin-Yan Li, Ce-Heng Liao, Qiong Wang, Yong-Jun Tan, Rui Luo, Ye Qiu, Xing-Yi Ge, The ORF6, ORF8 and nucleocapsid proteins of SARS-CoV-2 inhibit type I interferon signaling pathway, Virus Research, Volume 286, 2020, Khailany-Khailany R A, Safdar M, Ozaslan M. Genomic characterization of a novel SARS-CoV-2 [published online ahead of print, 2020 Apr. 16]. Gene Rep. 2020; 19:100682. doi:10.1016/j.genrep.2020.100682
T. Koyama, D. Platt, L. Panda, Variant analysis of COVID-19 genomes, Bull. World Health Organ. (2020)
Catanzaro, M., Fagiani, F., Racchi, M., Corsini, E., Govoni, S., Lanni, C., 2020. Immune response in COVID-19: addressing a pharmacological challenge by targeting pathways triggered by SARS-CoV-2. Signal Transduct. Target. Ther. 5 (1), 84.
Seema Mishra-Mishra, Seema (2020): ORF10: Molecular Insights into the Contagious Nature of Pandemic Novel Coronavirus 2019-nCoV. ChemRxiv. Preprint. https://doi.org/10.26434/chemrxiv.12118839.v3
Xu, J. et al., Viruses 2020, 12, 244
Tang, X. et al. National Science Review 2020, 7, 1012-1023
M. Bianchi et al, BioMed Research International Vol 2020 Article ID 4389089 Hassan S, Eldeeb K, Millns P J, Bennett A J, Alexander S P, Kendall D A.
Cannabidiol enhances microglial phagocytosis via transient receptor potential (TRP) channel activation. Br J Pharmacol. 2014; 171(9):2426-39.
da Silva V K, de Freitas B S, da Silva Dornelles A, Nery L R, Falavigna L, Ferreira R D, et al. Cannabidiol normalizes caspase 3, synaptophysin, and mitochondrial fission protein DNM1L expression levels in rats with brain iron overload: implications for neuroprotection. Molecular neurobiology. 2014; 49(1):222-33.
Huang Y, Wan T, Pang N, Zhou Y, Jiang X, Li B, et al. Cannabidiol protects livers against nonalcoholic steatohepatitis induced by high-fat high cholesterol diet via regulating NF-κB and NLRP3 inflammasome pathway. J Cell Physiol. 2019; 234(11):21224-34.
Hassan S, Eldeeb K, Millns P J, Bennett A J, Alexander S P, Kendall D A. Cannabidiol enhances microglial phagocytosis via transient receptor potential (TRP) channel activation. Br J Pharmacol. 2014; 171(9):2426-39.
da Silva V K, de Freitas B S, Domelles V C, Kist L W, Bogo M R, Silva M C, et al. Novel insights into mitochondrial molecular targets of iron-induced neurodegeneration: Reversal by cannabidiol. Brain Res Bull. 2018; 139:1-8.
Hao E, Mukhopadhyay P, Cao Z, Erdelyi K, Holovac E, Liaudet L, et al. Cannabidiol Protects against Doxorubicin-Induced Cardiomyopathy by Modulating Mitochondrial Function and Biogenesis. Mol Med. 2015; 21:38-45.
McKallip R J, Jia W, Schlomer J, Warren J W, Nagarkatti P S, Nagarkatti M. Cannabidiol-induced apoptosis in human leukemia cells: A novel role of cannabidiol in the regulation of p22phox and Nox4 expression. Mol Pharmacol. 2006; 70(3):897-908.
Ryan D, Drysdale A J, Lafourcade C, Pertwee R G, Platt B. Cannabidiol targets mitochondria to regulate intracellular Ca2+ levels. J Neurosci. 2009; 29(7):2053-63.
Valvassori S S, Bavaresco D V, Scaini G, Varela R B, Streck E L, Chagas M H, et al. Acute and chronic administration of cannabidiol increases mitochondrial complex and creatine kinase activity in the rat brain. Braz J Psychiatry. 2013; 35(4):380-6.
Jeong S, Jo M J, Yun H K, Kim D Y, Kim B R, Kim J L, et al. Cannabidiol promotes apoptosis via regulation of XIAP/Smac in gastric cancer. Cell Death Dis. 2019; 10(11):846.
Jeong S, Yun H K, Jeong Y A, Jo M J, Kang S H, Kim J L, et al. Cannabidiol-induced apoptosis is mediated by activation of Noxa in human colorectal cancer cells. Cancer letters. 2019; 447:12-23.
Sultan A S, Marie M A, Sheweita S A. Novel mechanism of cannabidiol-induced apoptosis in breast cancer cell lines. Breast. 2018; 41:34-41.
Oláh A, Markovics A, Szabó-Papp J, Szabó P T, Stott C, Zouboulis C C, et al. Differential effectiveness of selected non-psychotropic phytocannabinoids on human sebocyte functions implicates their introduction in dry/seborrhoeic skin and acne treatment. Exp Dermatol. 2016; 25(9):701-7.
Solinas M, Massi P, Cantelmo A R, Cattaneo M G, Cammarota R, Bartolini D, et al. Cannabidiol inhibits angiogenesis by multiple mechanisms. Br J Pharmacol. 2012; 167(6):1218-31.
Castillo A, Tolon M R, Fernandez-Ruiz J, Romero J, Martinez-Orgado J. The neuroprotective effect of cannabidiol in an in vitro model of newborn hypoxic-ischemic brain damage in mice is mediated by CB(2) and adenosine receptors. Neurobiology of disease. 2010; 37(2):434-40.
Sun S, Hu F, Wu J, Zhang S. Cannabidiol attenuates OGD/R-induced damage by enhancing mitochondrial bioenergetics and modulating glucose metabolism via pentose-phosphate pathway in hippocampal neurons. Redox Biol. 2017; 11:577-85.
Burstein S, Hunter S A, Renzulli L. Stimulation of sphingomyelin hydrolysis by cannabidiol in fibroblasts from a Niemann-Pick patient. Biochemical and biophysical research communications. 1984; 121(1):168-73.
Cornicelli J A, Gilman S R, Krom B A, Kottke B A. Cannabinoids impair the formation of cholesteryl ester in cultured human cells. Arteriosclerosis. 1981; 1(6):449-54.
Rimmerman N, Bradshaw H B, Kozela E, Levy R, Juknat A, Vogel Z. Compartmentalization of endocannabinoids into lipid rafts in a microglial cell line devoid of caveolin-1. Br J Pharmacol. 2012; 165(8):2436-49.
Lu Y et al 2008-45. Lu Y, Liu D X, Tam J P. Lipid rafts are involved in SARS-CoV entry into Vero E6 cells. Biochemical and biophysical research communications. 2008; 369(2):344-9.
Duncan R E, El-Sohemy A, Archer M C. Mevalonate promotes the growth of tumors derived from human cancer cells in vivo and stimulates proliferation in vitro with enhanced cyclin-dependent kinase-2 activity. The Journal of biological chemistry. 2004; 279(32):33079-84.
Duncan R E, Lau D, El-Sohemy A, Archer M C. Geraniol and beta-ionone inhibit proliferation, cell cycle progression, and cyclin-dependent kinase 2 activity in MCF-7 breast cancer cells independent of effects on HMG-CoA reductase activity. Biochemical pharmacology. 2004; 68(9):1739-47.
Gordon, D. E., Jang, G. M., Bouhaddou, M. et al. A SARS-CoV-2 protein interaction map reveals targets for drug repurposing. Nature 583, 459-468 (2020). https://doi.org/10.1038/s41586-020-2286-9
Bizzotto J, Sanchis P, Abbate M, Lage-Vickers S, Lavignolle R, Toro A, Olszevicki S, Sabater A, Cascardo F, Vazquez E, Cotignola J, Gueron G. SARS-CoV-2 Infection Boosts MX1 Antiviral Effector in COVID-19 Patients. iScience. 2020 Oct. 23; 23(10):101585.
Zhou S, Butler-Laporte G, Nakanishi T, Morrison D R, Afilalo J, Afilalo M, Laurent L, Pietzner M, Kerrison N, Zhao K, Brunet-Ratnasingham E, Henry D, Kimchi N, Afrasiabi Z, Rezk N, Bouab M, Petitjean L, Guzman C, Xue X, Tselios C, Vulesevic B, Adeleye O, Abdullah T, Almamlouk N, Chen Y, Chasse M, Durand M, Paterson C, Normark J, Frithiof R, Lipcsey M, Hultstrom M, Greenwood C M T, Zeberg H, Langenberg C, Thysell E, Pollak M, Mooser V, Forgetta V, Kaufmann D E, Richards J B. A Neanderthal OAS1 isoform protects individuals of European ancestry against COVID-19 susceptibility and severity. Nat Med. 2021 Feb. 25.
Duncan, R. E., et al., Geraniol and beta-ionone inhibit proliferation, cell cycle progression, and cyclin-dependent kinase 2 activity in MCF-7 breast cancer cells independent of effects on HMG-CoA reductase activity. Biochem Pharmacol, 2004. 68(9): p. 1739-47.
M'Hiri I, Diaguarachchige De Silva K H, Duncan R E. Relative expression and regulation by short-term fasting of lysophosphatidic acid receptors and autotaxin in white and brown adipose tissue depots. Lipids. 2020; 55(3):279-84.
Nagarkatti P, Pandey R, Rieder S A, Hegde V L, Nagarkatti M. Cannabinoids as novel anti-inflammatory drugs. Future Med Chem. 2009; 1(7):1333-1349. doi:10.4155/fmc.09.93.
Pascarella, S., et al., SARS-CoV-2 B.1.617 Indian variants: are electrostatic potential changes responsible for a higher transmission rate? J Med Virol, 2021.
Lazarevic, I., et al., Immune Evasion of SARS-CoV-2 Emerging Variants: What Have We Learnt So Far? Viruses, 2021. 13(7).
Cerutti, G., et al., Structural basis for accommodation of emerging B.1.351 and B.1.1.7 variants by two potent SARS-CoV-2 neutralizing antibodies. Structure, 2021. 29(7): p. 655-663.e4.
Kemp, S. A., et al., Neutralising antibodies in Spike mediated SARS-CoV-2 adaptation. medRxiv, 2020.

1-23. (canceled)

24. A method of treating Covid-19 infectious disease caused by Sars-Cov-2 virus in a patient wherein the said method comprises administering to said patient, a pharmaceutical composition comprising therapeutically effective amount of Cannabinoid wherein the administration of said pharmaceutical composition to the said patient produces an enhancement/augmentation of innate immunity of the patient due to at least one of the following effects,
i) infected patient cells undergo apoptosis early after infection;
ii) induction of interferon transcription in the patient;
iii) induction of interferon induced antiviral effector in the patient.

25. The method of treating Covid-19 infectious disease according to the claim 24 wherein the enhancement/augmentation of innate immunity of the patient is due to the apoptosis of infected patient cells early after infection which renders them not available to the virus for replication and/or mutation.

26. The method of treating Covid-19 infectious disease according to the claim 24 wherein the enhancement/augmentation of innate immunity of the patient is due to an early apoptosis or a late apoptosis or both an early and late apoptosis of the infected cells of the patient.

27. The method of treating Covid-19 infectious disease according to the claim 24 wherein the enhancement/augmentation of innate immunity of the patient is due to the apoptosis of infected patient cells early after infection thereby reducing or abolishing the ability of the virus to evade host immunity.

28. The method of treating Covid-19 infectious disease according to the claim 24 wherein the enhancement/augmentation of innate immunity of the patient is due to induction of interferon transcription in the said patient providing an innate, intracellular, anti-viral defense.

29. The method of treating Covid-19 infectious disease according to the claim 28 wherein the enhancement/augmentation of innate immunity of the patient is due to induction of Type II (gamma) or Type III (lambda) or both Type II and Type III Interferon transcription in such patients.

30. The method of treating Covid-19 infectious disease according to the claim 24 wherein the enhancement/augmentation of innate immunity of the patient is due to induction of interferon-induced antiviral effector in the patient wherein the antiviral effector is one or more of OAS1, OAS2, OAS3, OASL, Mx1 and IFIT1 genes.

31. A method of prophylaxis or prophylactic treatment of Covid-19 infectious disease caused by Sars-Cov-2 virus wherein said method comprises administering a pharmaceutical composition comprising therapeutically effective/prophylactically effective amount of a Cannabinoid to an animal/human wherein administration of said pharmaceutical composition produces an enhancement/augmentation of innate immunity in such animal/human due to at least one of the following effects,
i) infected animal or human cells undergo apoptosis early after infection;
ii) induction of interferon transcription in the animal/human;
iii) induction of interferon-induced antiviral effectors in the animal/human.

32. The method of prophylaxis or prophylactic treatment of the claim 31 wherein enhancement/augmentation of innate immunity in animal/human is not associated with apoptosis of cells.

33. The method of prophylaxis or prophylactic treatment of the claim 31 wherein enhancement/augmentation of innate immunity in animal/human is due to induction of Type II (gamma) or Type III (lambda) or both Type II and Type III Interferon transcription.

34. The method of prophylaxis or prophylactic treatment of the claim 31 wherein enhancement/augmentation of innate immunity in animal/human is due to induction of interferon induced antiviral effector wherein the antiviral effector is is one or more of OAS1, OAS2, OAS3, OASL, Mx1 and IFIT1 genes.

35. A method of preventing or reducing mutation of Sars-Cov-2 virus in an animal/human/patient wherein said method comprises administering a pharmaceutical composition comprising therapeutically effective amount of one or more Cannabinoid to said animal/human/patient exposed to/suffering from Covid-19 infectious disease by causing exposed human cells/infected patient cells to undergo apoptosis early after exposure/infection which renders them not available to the virus for mutation.

36. A method of better preparing an animal or human for Covid-19 infectious disease wherein said animal/human is about to get infected with Covid-19 infectious disease by administration to the said animal/human a pharmaceutical composition comprising therapeutically effective amount of one or more Cannabinoids wherein administration of said pharmaceutical composition produces an enhancement/augmentation of innate immunity in such animal/human due to at least one of the following effects,
i) infected animal or human cells undergo apoptosis early after infection;
ii) induction of interferon transcription in the animal/human;
iii) induction of interferon-induced antiviral effectors in the animal/human.

37. A method of treating or a method of prophylaxis or prophylactic treatment for Covid-19 infectious disease caused by Sars-Cov-2 virus wherein the said method comprises administering to said animal/human/patient, a pharmaceutical composition comprising prophylactically/therapeutically effective amount of Cannabinoid wherein the administration of said pharmaceutical composition to the said animal/human/patient produces an enhancement/augmentation of innate immunity of the animal/human/patient due to at least one of the following effects,
i) infected cells undergo apoptosis early after infection;
ii) induction of interferon transcription in the patient;
iii) induction of interferon induced antiviral effector in the patient;
and wherein one or more of the above effects causes at least one of the following,
i) clearing viruses partially or completely;
ii) preventing the development of infection, and raising the infectious titre needed to cause disease;
iii) preventing viral replication, and therefore mutant (variant) formation.

38. The method according to claim 24 wherein the Cannabinoid is selected from one or more of Cannabidiol, Cannabigerol, Cannabinol, Cannabidiolic acid, d8-Tetrahydrocannabivarin (d8-THCV).
